(19) United States
(12) Patent Application Publication
(10) Pub. No.: US 2026/0108637 A1
(43) Pub. Date: Apr. 23, 2026

(54) USES OF PSMA-TARGETING AND TSPO-TARGETING COMPOUNDS FOR EVALUATION OF INJURIES IN THE PERIPHERAL NERVOUS SYSTEM

(71) Applicant: The Johns Hopkins University, Baltimore, MD (US)

(72) Inventors: Martin Gilbert Pomper, Baltimore, MD (US); Catherine Foss, Baltimore, MD (US); Sami Tuffaha, Baltimore, MD (US); William Michael Padoveno, Baltimore, MD (US)

(21) Appl. No.: 19/405,641

(22) Filed: Dec. 2, 2025

Related U.S. Application Data
(63) Continuation-in-part of application No. PCT/US2024/032262, filed on Jun. 3, 2024.
(60) Provisional application No. 63/505,874, filed on Jun. 2, 2023.

(51) Int. Cl. A61K 51/04; A61K 49/00

(52) U.S. Cl. CPC A61K 51/0459; A61K 49/0034; A61K 49/0052; A61K 51/0455; A61K 51/0497; A61K 2123/00

(57) ABSTRACT

Methods for diagnosing a peripheral nervous system (PNS) neuropathy comprising administering to a subject in need of treatment thereof, at least one of a translocator protein (TSPO)-targeting compound or a PSMA-targeting compound and taking an image, are disclosed.

### CROSS-REFERENCE TO RELATED APPLICATIONS

This application is a continuation in part of International PCT Patent Application No. PCT/US2024/032262, filed Jun. 3, 2024, which claims priority to U.S. Provisional Application No. 63/505,874, filed Jun. 2, 2023, each of which is incorporated herein by reference in its entirety.

### FEDERALLY SPONSORED RESEARCH OR DEVELOPMENT

This invention was made with Government support under grant numbers CA134675, EB009367, EB024495, HL116316, HL131829 and OD006492, awarded by the National Institutes of Health and grant number 1397119 awarded by the Department of Defense. The government has certain rights in the invention.

### BACKGROUND

Peripheral nerve injuries (PNIs) are a common cause of permanent disability, pain, and reduced quality of life. Bailey et al., 2009. These injuries occur in an estimated 1% to 3% of extremity traumas in the United States, resulting in an estimated 70,000 to 150,000 nerve injuries per year. Padovano et al., 2022; Taylor et al., 2008. PNIs are even more prevalent among military service members and veterans, comprising 8% of all combat-related injuries. Birch et al., 2012(a). Despite increased awareness of these injuries and advances in nerve surgical techniques, functional recovery is generally poor. Indeed, 41% of patients with median or ulnar nerve injuries do not return to work within one year of injury, Bruyns et al., 2003, and patients with nerve injuries experience disproportionate disability relative to those with non-nerve extremity traumas. Birch et al., 2012(b).

Poor outcomes after appropriately treated PNIs are generally due to irreversible denervation-induced muscle atrophy rather than a failure of nerve regeneration. Sarhane et al., 2021. This degenerative process completes after around 18-24 months and nerve fibers reaching the muscle after this point are generally unable to animate the atrophic muscle. MacDonald et al., 2014; Ehmsen et al., 20202. Unfortunately, because no imaging modality can reliably characterize internal architecture, closed nerve injuries (or open injuries without overt epineurial discontinuity), are usually managed with months of “watchful waiting” to monitor for signs of spontaneous recovery. Months can be squandered, and permanent function lost, monitoring injuries that will never spontaneously recover.

To this end, assessment of nerve injuries generally involves physical examination and electrodiagnostic studies (EDX), which include electromyography (EMG) and nerve conduction studies (NCS). While EDX can often identify that a nerve pathology is present, it has limited utility for predicting outcomes or selecting surgical management. Robinson, 2015. This limitation arises because electrodiagnostic studies do not provide structural information on the nerve and therefore cannot distinguish between, for example, an axonotmetic injury (Sunderland Grade II) with a favorable prognosis and a neurotmetic injury (Sunderland Grade V) with a poor prognosis. Moreover, EDX provides only a rough estimate of an injury's location and no information on the zone of injury. Diagnostic high-resolution ultrasound can be a useful adjunct for evaluating structural compressive injuries, such as in carpal tunnel syndrome, but its poor spatial resolution also prevents assessment of internal nerve structure and zone of injury. Toros et al., 2009; Cartwright et al., 2007.

Recently, there has been academic interest in magnetic resonance diffusion tensor imaging (MR DTI) to characterize peripheral nerves. Pridmore et al., 2021. MR DTI can theoretically identify axonal injuries by pinpointing disruptions in axoplasmic flow along a nerve. Unfortunately, several practical factors limit its widespread clinical utility. Jeon et al., 2018. These factors include low signal-to-noise ratio, Widjaja et al., 2009, a lack of normative values and high variability in DTI values between patients, Hiltunen et al., 2005; Zhou et al., 2014, challenges imaging around orthopaedic hardware, Hargreaves et al., 2011, and very high susceptibility to motion artifacts including from pulsating arteries near nerves. Jones and Pierpaoli, 2005.

### SUMMARY

In some aspects, the presently disclosed subject matter provides a method for diagnosing a peripheral nervous system (PNS) neuropathy, wherein PNS pathology encompasses PNS insults and/or neuropathies, recovery of PNS insults and/or neuropathies, muscle denervation including muscle denervation-induced muscle atrophy, and/or muscle re-innervation. In particular aspects, the method comprises administering to a subject in need of treatment thereof, at least one of a translocator protein (TSPO)-targeting compound or a prostate-specific membrane antigen (PSMA)-targeting compound and taking an image. The imaging can include optical imaging, single-photon emission computerized tomography (SPECT) imaging, positron emission tomography (PET) imaging and/or magnetic resonance imaging (MRI).

In certain aspects, the TSPO-targeting compound comprises a compound of formula (I):

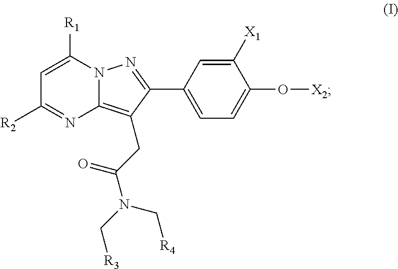

wherein: X₁ can be present or absent and when present is selected from a radioisotope of fluorine, a radioisotope of iodine, a radioisotope of bromine, and a radioisotope of astatine; under the proviso that when X₁ is present, X₂ is H or C₁-C₄ alkyl and when X₁ is absent, X₂ is -L-I, wherein L is a linker and I is an imaging agent; and R₁, R₂, R₃ and R₄ are each independently selected from hydroxyl, C₁ to C₁₀ alkyl, cycloalkyl, aryl, alkylamino, alkylamino, alkenyl, alkynyl, hydroxyalkyl, alkoxyl, dialkylamino thioalkyl, thioalkenyl, thioalkynyl, aryloxy, acyloxy, thioacyl, amido, and sulphonamido; wherein each of alkyl, or aryl moiety may be unsubstituted or substituted with one or more substituents selected from the group consisting of halo, hydroxyl, carboxyl, phosphoryl, phosphonyl, phosphono C₁-C₆ alkyl, carboxy C₁-C₆ alkyl, dicarboxy C₁-C₆ alkyl, dicarboxy halo C₁-C₆ alkyl, sulfonyl, cyano, nitro, alkoxy, alkylthio, acyl, acyloxy, thioacyl, acylthio, aryloxy, amino, alkylamino, dialkylamino, trialkylamino, arylalkylamino, guanidino, aldehydo, ureido, and aminocarbonyl, an amino acid residue, and a substituted amino acid residue; and pharmaceutically acceptable salts thereof.

In some aspects, the PSMA-targeting compound is a compound of formula (II):

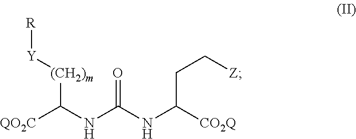

wherein: Z is tetrazole or CO₂Q; each Q is independently selected from hydrogen or a protecting group; and wherein:
(A) m is 0, 1, 2, 3, 4, 5, or 6;
R is a pyridine ring selected from:

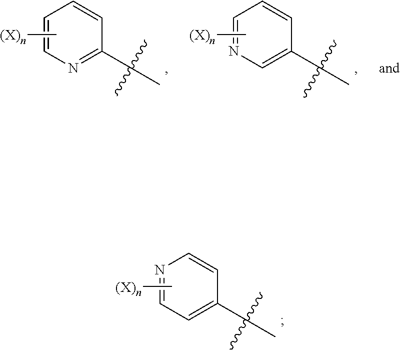

wherein:
X is fluorine, iodine, a radioisotope of fluorine, a radioisotope of iodine, chlorine, bromine, a radioisotope of bromine, a radioisotope of astatine, NO₂, NH₂, N⁺(R²)₃, Sn(R²)₃, Si(R²)₃, Hg(R²), B(OH)₂, —NHNH₂, —NHN═CHR³, —NHNH—CH₂R³;
n is 1, 2, 3, or 4;
Y is O, S, N(R′), C(O), NR′C(O), C(O)N(R′), OC(O), C(O)O, NR′C(O)NR′, NR′C(S)NR′, NR′S(O)₂, S(CH₂)ₚ, NR′(CH₂)ₚ, O(CH₂)ₚ, OC(O)CHR⁸NHC(O), NHC(O)CHR⁸NHC(O), or a covalent bond; wherein p is 1, 2, or 3, R′ is H or C₁-C₆ alkyl, and R⁸ is hydrogen, alkyl, aryl or heteroaryl, each of which may be substituted;
R² is C₁-C₆ alkyl; and
R³ is alkyl, alkenyl, alkynyl, aryl, or heteroaryl each of which is substituted by fluorine, iodine, a radioisotope of fluorine, a radioisotope of iodine, chlorine, bromine, a radioisotope of bromine, or a radioisotope of astatine, NO₂, NH₂, N⁺(R²)₃, Sn(R²)₃, Si(R²)₃, Hg(R²), or B(OH)₂; or
(B) m is 0, 1, 2, 3, 4, 5, or 6;
Y is O, S, N(R′), C(O), NR′C(O), C(O)N(R′), OC(O), C(O)O, NR′C(O)NR′, NR′C(S)NR′, NR′S(O)₂, S(CH₂)ₚ, NR′(CH₂)ₚ, O(CH₂)ₚ, OC(O)CHR⁸NHC(O), NHC(O)CHR⁸NHC(O), or a covalent bond; wherein p is 1, 2, or 3, R′ is H or C₁-C₆ alkyl, and R⁸ is hydrogen alkyl, aryl or heteroaryl, each of which may be substituted;
R is:

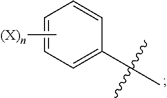

wherein:
X′ is selected from the group consisting of NHNH₂, —NHN═CHR³, and —NHNH—CH₂R³; wherein R³ is alkyl, alkenyl, alkynyl, aryl, or heteroaryl each of which is substituted by fluorine, iodine, a radioisotope of fluorine, a radioisotope of iodine, bromine, a radioisotope of bromine, or a radioisotope of astatine; NO₂, NH₂, N⁺(R²)₃, Sn(R²)₃, Si(R²)₃, Hg(R²), or B(OH)₂;
R² is C₁-C₆ alkyl;
n is 1, 2, 3, 4, or5; or
(C) m is 4;
Y is NR′; and
R is:

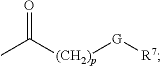

wherein G is 0, NR′ or a covalent bond;
R is H or C₁-C₆ alkyl; p is 1, 2, 3, or 4, and
R⁷ is selected from the group consisting of NH₂, N═CHR³, NH—CH₂R³, wherein R³ is alkyl, alkenyl, alkynyl, aryl, or heteroaryl each of which is substituted by fluorine, iodine, a radioisotope of fluorine, a radioisotope of iodine, chlorine, bromine, a radioisotope of bromine, or a radioisotope of astatine, NO₂, NH₂, N⁺(R²)₃, Sn(R²)₃, Si(R²)₃, Hg(R²), B(OH)₂; and R² is C₁-C₆ alkyl.

In other aspects, the PSMA-targeting compound comprises a compound of formula (III):

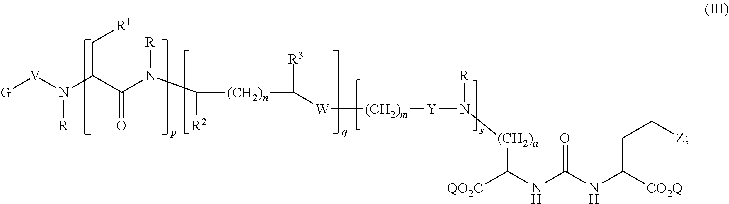

wherein:
q and s are each independently 0 or 1;
p is 0, 1, 2, or 3;
a is 1, 2, 3, or 4;
m is 1, 2, 3, 4, 5, or 6;
n is 1, 2, 3, 4, 5 or 6;
Z is tetrazole or CO₂Q;
each Q is independently selected from hydrogen or a protecting group;
V can be present or absent and when is present is selected from —C(O)—, —NRC(O)—, and —NRC(S)—;
W is selected from —NRC(O)—, —NRC(O)NR—, NRC(S)NR—, —NRC(O)O—, —OC(O)NR—, —OC(O)—, —C(O)NR—, or —C(O)O—;
Y is selected from —C(O)—, —NRC(O)—, —NRC(S)—, and —OC(O);
each R is independently H or C₁-C₄ alkyl;
each R¹ is independently H, C₁-C₆ alkyl, C₂-C₁₂ aryl, or C₄-C₁₆ alkylaryl, whereas when p is 2 or 3, each R¹ may be the same or different;
R² and R³ are independently H, CO₂H, or CO₂R⁴, where R⁴ is a C₁-C₆ alkyl, C₂-C₁₂ aryl, or C₄-C₁₆ alkylaryl, wherein when one of R² and R³ is CO₂H or CO₂R⁴, the other is H; and
G is a metal chelating moiety optionally including a chelated metal or a dye moiety that emits in the visible or near infrared spectrum, wherein G can include any additional atoms or linkers necessary to attach the metal chelating moiety or dye moiety to the rest of the compound.

In other aspects, the PSMA-targeting compound is a compound of formula (IV):

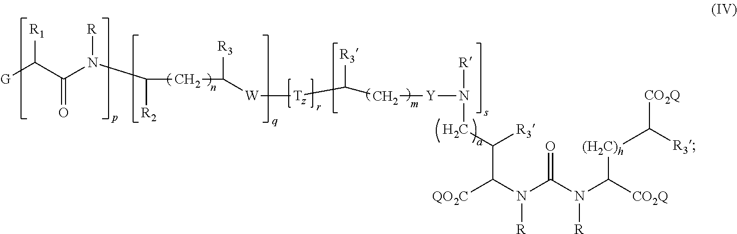

wherein:
each Q is independently hydrogen, a metal ion, a negative charge, or a protecting group;
a, h, m, and n are each independently an integer selected from the group consisting of 1, 2, 3, 4, 5, and 6;
s is 0 or 1;
r is 0 or 1;
q is 0 or 1;
p is an integer selected from the group consisting of 0, 1, 2, and 3, and when p is 2 or 3, each R and R₁ can be the same or different;
each R, R′, and R₁ is independently hydrogen, C₁-C₆ substituted or unsubstituted alkyl, C₆-C₁₂ substituted or unsubstituted aryl, C₆-C₁₂ substituted or unsubstituted heteroaryl, or C₆-C₁₆ alkyaryl;
R₂, R₃, and R₃′ are each independently hydrogen, C₁-C₄ substituted or unsubstituted alkyl, —CO₂H, —CO₂Q, or —CO₂R₄, wherein R₄ is a C₁-C₆ substituted or unsubstituted alkyl, C₆-C₁₂ substituted or unsubstituted aryl, C₆-C₁₂ substituted or unsubstituted heteroaryl, or C₆-C₁₆ alkyaryl, wherein if one of R₂ and R₃ is —CO₂H, or —CO₂R₄, then the other is H;
Tz is a triazole containing moiety selected from the group consisting of:

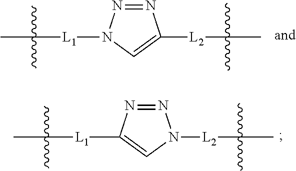

wherein: L₁ is

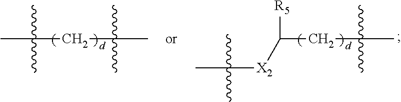

and L₂ is

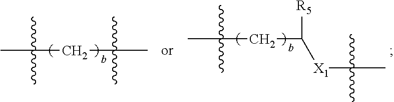

wherein:
X₁ is —NRC(O)—, —NRC(O)NR—, —NRC(S)NR, or —NRC(O)O—;
X₂ is —C(O)NR—, —NRC(O)NR—, —NRC(S)NR—, or —OC(O)NR—;
R₅ is H, —CO₂H, or —CO₂R⁶, wherein R₆ is C₁-C₆ alkyl, C₆-C₁₂ aryl, or C₆-C₁₆ alkyaryl; b is 1, 2, 3, or 4; and d is 1, 2, 3, or 4;
Y is —C(O)—, —NRC(O)—, —NRC(S)—, —OC(O)—;
W is a bond, —(CH₂—O)ₜ—, —NRC(O)—, —NRC(O)NR—, —NRC(S)NR—,
—NRC(O)O—, —OC(O)NR—, —OC(O)—, —C(O)NR—, or —C(O)O—, wherein t is an integer selected from the group consisting of 1, 2, 3, 4, 5, 6, 7, and 8;
G is

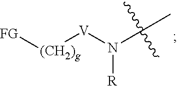

FG is a fluorescent dye moiety that emits in the near-infrared spectrum; V is —C(O)—, —NRC(O)—, —NRC(S)—, or —OC(O)—, and g is an integer selected from the group consisting of 1, 2, 3, 4, 5, and 6;
under the condition that when r is 0, then q and s are both 0 or both 1;
or a pharmaceutically acceptable salt thereof; under the proviso that if R′ is hydrogen.

Certain aspects of the presently disclosed subject matter having been stated hereinabove, which are addressed in whole or in part by the presently disclosed subject matter, other aspects will become evident as the description proceeds when taken in connection with the accompanying Examples and Drawings as best described herein below.

### BRIEF DESCRIPTION OF THE DRAWINGS

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying drawings, which are not necessarily drawn to scale, and wherein:

FIG. 1A, FIG. 1B, FIG. 1C, and FIG. 1D show fluorescently labeled DPA-713 (DPA-713-IRDye 680RD) uptake four weeks after sciatic nerve transection. FIG. 1A and FIG. 1B were taken from Animal 1 and FIG. 1C and FIG. 1D were taken from Animal 2 to allow for direct side-by-side comparisons. (FIG. 1A) control, non-operated leg Animal 1; (FIG. 1B) four weeks after sciatic nerve transection. Note DPA-713-dye uptake (red) in the distal sciatic nerve stump and common peroneal nerve; (FIG. 1C) control, non-operated leg Animal 2; and (FIG. 1D) four weeks after sciatic nerve transection; lateral gastrocnemius muscle is cut and reflected to expose the tibial nerve. Note increased DPA-713-dye uptake in the common peroneal, tibial, and sural nerves.

FIG. 2A, FIG. 2B, FIG. 2C, and FIG. 2D show fluorescently labeled DPA-713 (DPA-713-IRDye 680RD) uptake after sciatic nerve repair. FIG. 2A and FIG. 2B were taken from Animal 3 and FIG. 2C and FIG. 2D were taken from Animal 4 to allow for direct side-by-side comparisons. (FIG. 2A) two weeks after common peroneal nerve crush. Note increased uptake of DPA-713-dye in the common peroneal nerve relative to the sciatic and sural nerves; (FIG. 2B) two weeks after sciatic nerve transection and repair; lateral gastrocnemius muscle is cut and reflected to expose the tibial nerve. Note DPA-713-dye uptake distal to the nerve coaptation (repair) site; (FIG. 2C) control, non-operated leg Animal 4; and (FIG. 2D) four weeks after sciatic nerve transection and repair. Note increased DPA-713-dye uptake in the distal portion of the tibial nerve, consistent with the expected progress of nerve regeneration at this timepoint.

FIG. 3 shows rodent ex vivo biodistribution results: 49 adult Lewis rats after ¹²⁴I-iodo-DPA713 administration. Sciatic nerve uptake of ¹²⁴I-iodo-DPA713 over time is represented as percent injected dose per segment (% ID/segment) for sham, repaired nerve, and unrepaired nerve at 2, 4, and 16 weeks post-injury. In this example, the sciatic nerve was divided into 4 approximately equal segments: two proximal to the site of injury and two distal to the site of injury. These segments are numbered 1 through 4 in this figure. Uptake was calculated for each segment using an automated gamma counter. In the figure, surgical groups are represented as bars, which are overlapped. Purple color represents the overlap of red (i.e., unrepaired) and blue (i.e., repaired) groups.

FIG. 4 shows an axial slice of a reconstructed pig PET-CT: Yorkshire pig underwent left median nerve transection without repair and right median nerve with repair. At 16 weeks post-op, at a timepoint when no substantial muscle reinnervation is expected, the animal underwent ¹²⁴I-iodo-DPA713 PET-CT. Note increased uptake in distal forearm corresponding to anticipated location of median nerve.

FIG. 5 shows pig histology: Note elevated TSPO expression that co-localizes with CD68 at the site of nerve injury and distal in the unrepaired median nerve. CD68 is a common macrophage marker.

FIG. 6 shows human histology: Note elevated TSPO expression that co-localizes with CD68 in the distal portion of nerves after several months after nerve injury.

FIG. 7A, FIG. 7B, and FIG. 7C show 2-weeks post-operative YC27 uptake. Images taken after tail vein injection. Areas of interest circled in white. (FIG. 7A) Sciatic transection with repair (arrowhead). Note increased uptake in the gastrocnemius and anterior compartment muscles. (FIG. 7B) Common peroneal nerve crush. Note uptake in the anterior compartment muscles, which are innervated by the common peroneal nerve, and sparing of the gastrocnemius muscle, which is innervated by the uninjured tibial nerve. (FIG. 7C) Selective transection without repair of the nerve branch to the medial head of the gastrocnemius muscle. The gastrocnemius muscle is split to reveal the tibial nerve. Note uptake in the medial head with sparing of the uninjured lateral head of the gastrocnemius muscle. *Images and colorbar enhanced to improve visibility for reproduction using Microsoft PowerPoint V16.71 (brightness 35%, contrast 35%).

FIG. 8A, FIG. 8B, and FIG. 8C show 4-weeks post-operative YC27 uptake. Images taken after tail vein injection. Areas of interest circled in white. (FIG. 8A) Sciatic transection without repair (asterisk). Note increased uptake in the gastrocnemius and anterior compartment muscles. (FIG. 8B) Sciatic transection with repair (arrowhead). Note similar uptake in gastrocnemius and anterior compartment muscles as in FIG. 8A. (FIG. 8C) Common peroneal nerve crush. Note near-complete resolution of uptake at this timepoint, consistent with the expected course of muscle reinnervation after crush injury. *Images and colorbar enhanced to improve visibility for reproduction using Microsoft PowerPoint V16.71 (brightness 35%, contrast 35%).

FIG. 9A, FIG. 9B, and FIG. 9C show 16-weeks post-operative YC27 uptake. Images taken after tail vein injection. Areas of interest circled in white. (FIG. 9A) Sciatic transection without repair (asterisk). Note increased uptake in the gastrocnemius and anterior compartment muscles. There also is slight uptake in the proximal stump of the cut sciatic nerve, which by this timepoint has formed a mature neuroma. (FIG. 9B) Sciatic transection with repair (arrowhead). Note absence of elevated muscle uptake at this timepoint, consistent with the expected course of muscle reinnervation. (FIG. 9C) Common peroneal nerve crush. Note absence of elevated muscle uptake at this timepoint. *Images and colorbar enhanced to improve visibility for reproduction using Microsoft PowerPoint V16.71 (brightness 35%, contrast 35%).

FIG. 10 shows rodent ex vivo biodistribution results: 48 adult Lewis rats after ¹⁸F-DCFPyL administration. Groups consisted of sciatic nerve transection with repair, transection without repair, and sham surgery (uninjured nerve). Animals underwent injection and harvest at 2, 4, and 16 weeks. Mean gastrocnemius muscle uptake administration 2, 4, and 16 weeks after injury; Gray: sham surgery (uninjured nerve); Blue: repaired nerve; Red: unrepaired nerve; n=5-6 per group per timepoint. Note persistent muscle uptake in the unrepaired group over time.

FIG. 11 shows tissue [¹⁸F]DCFPyL uptake after blocking GCPII binding with ZJ-43 coadministration; sham surgery (uninjured nerve), pooled across all timepoints; Red: unrepaired nerve, pooled across all timepoints; Purple: ZJ-43 co-administration, 4 weeks after sciatic nerve transection without repair. One outlier in biceps muscle unrepaired group (1.2% ID/g) was excluded in the plot for improved visibility of remaining data; the outlier was included in statistical comparisons. Groupwise comparisons performed using Kruskal-Wallis test. % ID/g: percent injected dose per gram.

FIG. 12 shows an axial slice of a reconstructed rodent PET-MRI. 6 adult Lewis rats underwent serial ⁶⁸Ga-PSMA-11 PET-MRI at 4 and 16 weeks post-operative. Animals underwent sciatic transection with repair (n=3) or transection without repair (n=3). Example axial slice of animal 4 weeks after transection without repair. Note increased uptake in the atrophic denervated muscle.

FIG. 13 shows a summary of rodent PET-MRI results. Difference in ⁶⁸Ga-PSMA-11 uptake standardized uptake value (SUV) between rodent leg muscles on the operated side and non-operated side. For each animal, leg SUV was calculated as average uptake in kBq/cc and normalized by average testes uptake in kBq/cc. Note the same group of 6 animals underwent imaging at 4 and 16 weeks. Ratio of operative (right) hindlimb and non-operative (left) hindlimb activity in kBq/cc. Repeated measures ANOVA demonstrated a significant change in trend between unrepaired and repaired groups (p=0.037). Y-axis reference set to 1.0.

FIG. 14 shows axial, coronal, and sagittal slices of a reconstructive pig PET CT: Yorkshire pig underwent left median nerve transection without repair and right median nerve with repair. At 16 weeks post-operative, at a timepoint when no substantial muscle reinnervation is expected, the animal underwent ⁶⁸Ga-PSMA-11 PET-CT. Note increased uptake in median nerve innervated flexor muscles.

FIG. 15 shows pig histology: Note elevated GCPII expression that co-localizes with CD68 in denervated flexor carpi radialis muscle relative to innervated pectoralis major muscle. Alpha-BTX (alpha-bungarotoxin) antibody targets neuromuscular junctions. Beta-Tubulin antibody targets neurofilaments. CD68 is a common macrophage marker.

FIG. 16, Panels A-I, show rodent immunohistochemistry evaluating muscle innervation. Composite images of α-bungarotoxin (red), anti-neurofilament H (purple), anti-GCPII (green). (Panels A-D) Muscles reinnervation at progressive timepoints following nerve transection with immediate reconstruction: (Panel A) 4 weeks, (Panel B) 8 weeks, (Panel C) 12 weeks, (Panel D) 16 weeks. Note recovery of neurofilament staining by 8 weeks and normal endplate (red) shape by 16 weeks; note also GCPII expression around reinnervated endplates by 8 weeks. (Panels E-H) Denervated muscles at progressive timepoints following nerve transection without reconstruction: (Panel E) 4 weeks, (Panel F) 8 weeks, (Panel G) 12 weeks, (Panel H) 16 weeks. Note progressive flattening and fragmentation of endplates and a lack of GCPII expression near denervated endplates. (Panel I) Uninjured muscle; note overall similar appearance of endplates as in panel D. Confocal images at 20×, scale bar: 20 μm.

FIG. 17, Panels A-I, show rodent immunohistochemistry evaluating muscle GCPII expression. Composite images of anti-GCPII (green), anti-cytochrome C (red), DAPI (blue). (Panels A-D) Muscles reinnervation at progressive timepoints following nerve transection with immediate reconstruction: (Panel A) 2 weeks, (Panel B) 8 weeks, (Panel C) 12 weeks, (Panel D) 16 weeks. Note clusters of combined GCPII and cytochrome C staining (yellow) along the sarcolemma near nuclei (blue). (Panels E-H) Denervated muscles at progressive timepoints following nerve transection without reconstruction: (Panel E) 2 weeks, (Panel F) 8 weeks, (Panel G) 12 weeks, (Panel H) 16 weeks. Note sarcolemmal clusters of GCPII staining persist near nuclei, although the overall cytochrome C staining (red) declines over time. (Panel I) Uninjured muscle; note overall similar appearance as in panels B-D. Confocal images at 63×, scale bar: 10 μm.

FIG. 18, Panels A-I, show near-infrared images of YC27 hindlimb uptake after various nerve injuries. Disarticulated rat hindlimbs; the skin and biceps femoris muscles have been cut away to expose muscles. Areas of interest circled (white). (Panels A-C) 2 weeks post-injury: (Panel A) Selective transection of tibial nerve branch to the medial gastrocnemius muscle, resulting in YC27 uptake in the medial gastrocnemius muscle only; (Panel B) Sciatic transection and repair, resulting in uptake in anterior and posterior leg compartment muscles; (Panel C) Common peroneal nerve crush, resulting in uptake in the anterior leg compartment muscles. (Panels D-F) 4 weeks post-injury: (Panel D) Sciatic transection without repair, (Panel E) Sciatic transection with repair, (Panel F) Common peroneal nerve crush. Note persistent YC27 uptake after sciatic transection irrespective of repair and reduced uptake after common peroneal nerve crush. (Panels G-I) 16 weeks post-injury: (Panel G) Sciatic transection without repair, (Panel H) Sciatic transection with repair, (Panel I) Common peroneal nerve crush. Note significant muscle atrophy in unrepaired muscle (i.e., chronically denervated) and resolution of YC27 uptake in the repaired muscle (i.e., reinnervated) by this timepoint. Images taken on Pearl Impulse Near-Infrared Imager with white light background (White light channel settings: minimum intensity 5.90×10⁻⁵, maximum intensity 5.16, gamma: 2.26). Post-processing: brightness and contrast uniformly increased 30% on all images to improve visibility for reproduction (Microsoft PowerPoint V16.71).

FIG. 19 shows serial rat [⁶⁸Ga]PSMA-11 PET/MRI. The same six animals were imaged at week 4 and week 16 after right sciatic transection with or without repair. Example axial images at the level of the calf; right hindlimb circled. Panel i and panel ii are from the same animal (repaired) and panel iii and panel iv are from another animal (unrepaired) imaged at week 4 and week 16. Note reduced uptake in panel iv (repaired; week 16) relative to panel ii (repaired; week 4) and panel iii (unrepaired; week 16).

FIG. 20A, FIG. 20B, and FIG. 20C show pig PET/CT 16 weeks after bilateral median nerve injuries. Animal received 89.17 MBq (2.41 mCi) of [⁶⁸Ga]PSMA-11 1 hour prior to imaging (FIG. 20A) Axial view, forearms circled; (FIG. 20B) Sagittal view, forearm circled; (FIG. 20C) Coronal view. Note muscle uptake in denervated median nerve-innervated flexor muscles. Intensity expressed in kBq/cc.

FIG. 21A, FIG. 21B, and FIG. 21C show human PET/CT 15 weeks after left radial nerve injury. The patient had no clinical or electrodiagnostic evidence of radial nerve recovery at the time of imaging. (FIG. 21A) Axial view, left forearm circled; (FIG. 21B) Coronal view, left forearm circled; (FIG. 21C) Sagittal views of right and left arms. Note increased uptake in the left mobile wad compartment (brachioradialis, extensor carpi radialis longus, and extensor carpi radialis brevis muscles) and forearm extensor compartment muscles relative to the uninjured right side. Intensity in the left humerus is a result of humerus fracture and hardware for internal fixation.

FIG. 22A, FIG. 22B, FIG. 22C, FIG. 22D, and FIG. 22E show rodent immunohistochemistry demonstrating muscle innervation after nerve transection versus crush injuries. (FIG. 22A-FIG. 22C) β-Tubulin (green) in gastrocnemius muscle 4 weeks post-transection: (FIG. 22A) sham surgery, (FIG. 22B) sciatic transection without repair, (FIG. 22C) sciatic transection with repair. Note lack of β-Tubulin staining in both repaired and unrepaired animals at this timepoint. (FIG. 22D-FIG. 22E) Composite images of anti-β-Tubulin (green), α-bungarotoxin (red), and DAPI (blue) in tibialis anterior muscle after common peroneal nerve crush: (FIG. 22D) 2 weeks post-crush, note denervated motor endplate morphology; (FIG. 22E) 4 weeks post-crush, note recovery of co-localization between β-Tubulin and α-bungarotoxin and restoration of normal motor endplate morphology. Confocal images at 20×, scale bar: 100 μm.

FIG. 23A, FIG. 23B, and FIG. 23C show pig immunohistochemistry demonstrating muscle denervation 16 weeks after median nerve injury. The left median nerve was transected without repair and the right median nerve was transected and repaired. Composite images of α-bungarotoxin (red), anti-β-Tubulin (green), and DAPI (blue) staining. (FIG. 23A) Left flexor carpi radialis muscle, (FIG. 23B) Right flexor carpi radialis muscle; note lack of β-Tubulin staining at this timepoint irrespective of repair. (FIG. 23C) Pectoarlis major muscle as an uninjured reference. Note positive β-Tubulin staining and area of co-localization between β-Tubulin and alpha-bungarotoxin (yellow), demonstrating an innervated motor endplate.

FIG. 24 shows a current treatment paradigm for upper extremity reconstruction after spinal cord injury. Most patients are referred for extremity reconstruction more than 1 year after injury, due in part to a lack of accurate diagnostic modalities for muscle denervation. Even after surgical referral, poor accuracy in identifying muscle denervation from lower motor neuron (LMN) injuries complicates patient management.

FIG. 25 shows contemplated changes to SCI management paradigm due to improved diagnosis of lower motor neuron (LMN) injuries with [¹⁸F]DCFPyL PET/CT. Expedient, non-invasive, and accurate diagnosis of muscle denervation may improve early referrals and reduce surgical uncertainty.

FIG. 26A, FIG. 26B, and FIG. 26C illustrate [¹⁸F]DCFPyL PET/CT after C4-level SCI. This 21-year-old man sustained a traumatic SCI 16 months prior. He was interested in restoration of right elbow flexion. (FIG. 26A) Axial view at the level of the shoulder girdle. High [¹⁸F]DCFPyL uptake (green color) in C5-C6 innervated subscapularis (orange), infraspinatus (blue), and deltoid (red) muscles, suggesting denervation. The subclavian vessels (yellow) also demonstrate high intravascular activity from blood pool. The trapezius muscles (white) are innervated by the spinal accessory nerve (CN XI), which is not injured in this patient, and thus demonstrate low [¹⁸F]DCFPyL uptake as expected. (FIG. 26B) Axial view of the right upper arm demonstrating high uptake in both the elbow flexors (red) and triceps muscle (white). (FIG. 26C) By contrast, axial view of the left upper arm demonstrates high [¹⁸F]DCFPyL uptake in elbow flexors but low uptake in the left triceps (white), suggesting preserved triceps muscle innervation.

FIG. 27A and FIG. 27B show [¹⁸F]DCFPyL PET/CT after C6-level SCI. This 23-year-old man sustained a traumatic incomplete SCI 20 months prior. He is positioned with his arms over his head. The deltoid (blue), biceps/brachialis (red), and triceps (white) muscles are circled. Clinically, he had normal deltoid and biceps/brachialis function bilaterally. He also had normal strength in his right triceps but no notable function in his left triceps. (FIG. 27A) Axial view of his right upper arm demonstrates low PET uptake in his deltoid and biceps muscles. He had high PET uptake in his lateral triceps head (La) but otherwise has low uptake in the long (Lo) and medial (Me) triceps heads, explaining his clinically normal strength. (FIG. 27B) Axial view of his left upper arm demonstrating high PET uptake in all three triceps heads, consistent with muscle denervation and explaining absent clinical function.

FIG. 28 shows [¹⁸F]DCFPyL PET/CT after incomplete cervical SCI from a hemorrhagic arteriovenous malformation. This 59-year-old woman experienced the injury 7 months prior, which resulted in persistent absence of right elbow flexion. The biceps/brachialis muscles are circled in red and demonstrate high PET uptake, indicating muscle denervation as expected. The intercostal muscles at this level are circled in white and demonstrate low PET uptake, suggesting that innervation to these muscles is intact. The aortic arch also demonstrates high PET activity due to blood pool.

FIG. 29A, FIG. 29B, and FIG. 29C demonstrate that [¹⁸F]DCFPyL PET/CT assists in localization of lesions in Parsonage-Turner syndrome. This 56-year-old man with Parsonage-Turner syndrome appeared to have disease localized to his left radial nerve on clinical exam and electrodiagnostic testing. Sagittal views of the (FIG. 29A) right arm and (FIG. 29B) left arm demonstrate PET uptake in the left triceps muscle (white) and radial nerve-innervated forearm musculature (red). Coronal view (FIG. 29C) redemonstrates left triceps muscle uptake (white), as well as uptake in the left latissimus muscle (red). This observation suggests a lesion that is more proximal than the origin of the radial nerve, such as in the posterior cord of the brachial plexus.

### DETAILED DESCRIPTION

The presently disclosed subject matter now will be described more fully hereinafter with reference to the accompanying Drawings, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated drawings. Therefore, it is to be understood that the presently disclosed subject matter is not to be limited to the specific embodiments disclosed and that modifications and other embodiments are intended to be included within the scope of the appended claims.

The presently disclosed subject matter represents the first clinically viable way to non-invasively evaluate peripheral nerve demyelination and recovery. In some embodiments, the presently disclosed subject matter provides for the use of translocator protein (TSPO)-targeting compounds and/or PSMA-targeting compounds to characterize peripheral nerve injuries (PNIs) and nerve regeneration via, for example, optical imaging, single-photon emission computerized tomography (SPECT) imaging, positron emission tomography (PET) imaging, and/or magnetic resonance imaging (MRI).

Following PNI, a well-described process of cellular and structural changes termed Wallerian degeneration occurs in the nerve downstream from the site of injury. This process is marked by a robust influx of activated macrophages around 3-5 days after injury, myelin degradation, and conversion of Schwann cells from a myelinating to a non-myelinating regenerative phenotype. The infiltrated macrophages largely remain within the denervated distal nerve segment until they are progressively pushed out by a regenerating front of axons, provided that regeneration occurs. Therefore, visualizing macrophage content along an injured nerve may provide an inverse image of nerve regeneration.

TSPO is highly expressed in activated macrophages and microglia in neuroinflammatory conditions. Because TSPO is expressed in high concentrations on macrophages and non-myelinating regenerative phenotype Schwann cells, TSPO-targeting compounds may be used to characterize nerve injuries. Non-invasive evaluation of PNIs could fundamentally change management of these devastating injuries by allowing for earlier and more accurate diagnoses, better-informed care discussions, and improved monitoring of recovery.

Considering the severe consequences of peripheral nerve injury, the lack of alternative diagnostic modalities, and the morbidity associated with delayed or inadequate surgical management, the presently disclosed subject matter represents a ground-breaking improvement over current products and many issues associated with diagnosing PNIs may be overcome by augmenting, for example, extremity MRI with PET imaging.

In other embodiments, the presently disclosed subject matter provides for the use of PSMA-targeting compounds to characterize muscle denervation and reinnervation, such as via positron emission tomography (PET). This embodiment represents a novel use for these compounds and a potential breakthrough for the diagnosis and management of conditions associated with peripheral nervous system (PNS) neuropathy, including peripheral nerve injury (PNI), brachial plexus injury (BPI), and spinal cord injury (SCI).

PSMA, also referred to as glutamate carboxypeptidase II (GCPII) or N-acetyl-L-aspartyl-L-aspartyl-L-glutamate peptidase I (NAALADase 1), converts N-acetylaspartyl-glutamate (NAAG) to N-acetyl-aspartate (NAA) and glutamate. PSMA is typically expressed in very low levels in the muscle. A recent rodent study on amyotrophic lateral sclerosis (ALS), however, identified increased PSMA expression in affected muscles, Szabo et al, 2015, which was attributed to an influx of a specific population of inflammatory macrophages. Tallon et al., 2022. While macrophages infiltrate denervated muscle within 3-5 days of injury and are thought to modulate denervated neuromuscular junctions (NMJs), the purpose for this increased PSMA expression is not currently known. Glutamate appears to play a critical role in NMJ maturation during development, and blocking PSMA delays synaptic pruning. A similar role, however, has not been explored in adult animals. The presently disclosed subject matter is the first to demonstrate increased uptake of PSMA-targeting ligands in denervated muscle after nerve injury, as well as resolution of uptake with muscle reinnervation. To this end, imaging with PSMA-targeting ligands may be used to diagnose PNS neuropathy and monitor recovery after treatment.

More particularly, in some embodiments, the presently disclosed subject matter provides a method for diagnosing a peripheral nervous system (PNS) neuropathy, the method comprising administering to a subject in need of treatment thereof, at least one of a translocator protein (TSPO)-targeting compound or a PSMA-targeting compound and taking an image. The imaging can include optical imaging, single-photon emission computerized tomography (SPECT) imaging, positron emission tomography (PET) imaging and/or magnetic resonance imaging (MRI). In certain embodiments, the imaging is in vitro, in vivo, or ex vivo. In some embodiments, the method further comprises diagnosing based on the image a disease or condition in a subject. In some embodiments, the method further comprises monitoring, based on the image, progression or regression of a disease or condition in a subject.

As used herein the term “PNS pathology” encompasses PNS insults and/or neuropathies, recovery of PNS insults and/or neuropathies, muscle denervation including muscle denervation-induced muscle atrophy, and/or muscle re-innervation. In certain embodiments, the peripheral nervous system (PNS) neuropathy is selected from a peripheral nerve injury (PNI), a brachial plexus injury (BPI), and a spinal cord injury (SCI).

Generally, a peripheral nerve fiber contains an axon (or long dendrite), myelin sheath, their Schwann cells, and the endoneurium. Peripheral nerve injury can be classified as: neurapraxia, in which the nerve remains intact but signaling ability is damaged; axonotmesis, in which the axon is damaged but the surrounding connecting tissue remains intact; and neurotmesis, in which both the axon and connective tissue are damaged. Neurapraxia is a temporary interruption of conduction without loss of axonal continuity. In neurapraxia, there is a physiologic block of nerve conduction in the affected axons. The endoneurium, perineurium, and the epineurium are intact and there is no Wallerian degeneration. Conduction is intact in the distal segment and proximal segment, but no conduction occurs across the area of injury. Axonotmesis involves loss of the relative continuity of the axon and its covering of myelin, but preservation of the connective tissue framework of the nerve (the encapsulating tissue, the epineurium and perineurium, are preserved). Wallerian degeneration occurs distal to the site of injury and there are sensory and motor deficits distal to the site of lesion and there is no nerve conduction distal to the site of injury (up to 3 to 4 days after injury). Neurotmesis is a total severance or disruption of an entire nerve fiber. Neurotmesis may be partial or complete. Wallerian degeneration occurs distal to the site of injury. In neurotmesis, sensory-motor problems and autonomic function defect are severe. There is no nerve conduction distal to the site of injury (3 to 4 days after lesion). Surgical intervention typically is necessary.

More particularly, PNIs can be classified under two systems, the Seddon Classification and the Sunderland Classification. The Seddon Classification classifies peripheral nerve injuries into neuropraxia, axonotmesis, and neurotmesis. The Sunderland Classification further classifies PNIs into different degrees or levels of injury.

Neurapraxia is usually secondary to compression pathology and is the mildest form of peripheral nerve injury with minimal structural damage. Neurapraxia typically allows for a complete recovery with a relatively short recovery period. In a neuropraxic injury, a focal segment of the nerve is demyelinated at the site of injury with no injury or disruption to the axon or its surroundings. This type of PNI is usually due to prolonged ischemia from excess pressure or stretching of the nerve with no Wallerian degeneration. Symptoms associated with neurapraxia include pain, little to no muscle wasting, muscle weakness, numbness, and proprioception issues. Neurapraxia falls under a first degree Sunderland Classification.

Axonotmesis involves damage to the axon and its myelin sheath. The endoneurium, perineurium, and epineurium, however, remain intact. Although the internal structure is preserved, the damage of the axons does lead to Wallerian degeneration. This type of nerve injury also results in a complete recovery although it does take longer than a neuropraxic injury. Symptoms associated with axonotmesis is manifested with pain, muscle wasting, complete motor, sensory, and sympathetic function loss. Axonotmesis falls under the second and third degree Sunderland Classification.

Neurotmesis can occur at different levels and can be characterized using the Sunderland classification system. A 3rd-degree neurotmesis injury involves the disruption of the axon and endoneurium, in which the perineurium and epineurium remain intact. Disruption of the axon and perineurium is considered a 4th-degree injury. A complete disruption of the entire nerve trunk is classified as a 5th-degree injury.

In some embodiments, the peripheral nerve injury (PNI) is selected from a stretch-related PNI, a laceration, a compression PNI, and a PNI related to one or more conditions selected from radiation, electricity, injection, crush, cold, and an intra-neural or extra-neural pathology.

Representative PSMA-targeting compounds and TSPO-targeting compounds are provided in:
International PCT Patent Application Publication No. WO2010/108125 for PSMA-Targeting Compounds and Uses Thereof, to Pomper et al., published Sep. 23, 2010.
U.S. Pat. No. 9,056,841 for PSMA-Targeting Compounds and Uses Thereof, to Pomper et al., issued Jun. 16, 2015;
U.S. Pat. No. 9,776,977 for PSMA-Targeting Compounds and Uses Thereof, to Pomper et al., issued Oct. 3, 2017;
U.S. Pat. No. 10,717,750 for ⁶⁸Ga-labeled NOTA-chelated PSMA-targeted Imaging and Therapeutic Agents, to Pomper et al., issued Jul. 21, 2020;
U.S. Pat. No. 11,021,450 for PSMA Targeted Fluorescent Agents for Image Guided Surgery, to Pomper et al., issued Jun. 1, 2021;
U.S. Pat. No. 11,661,402 for PSMA Targeted Fluorescent Agents for Image Guided Surgery, to Pomper et al., issued Mar. 30, 2023;
U.S. Patent Application Publication No. 2022/0048872 for PSMA Targeted Fluorescent Agents for Image Guided Surgery, to Pomper et al., published Feb. 17, 2022;
International PCT Patent Application Publication No. WO2010/014933 for PSMA-Binding Agents and Uses Thereof, to Pomper et al., published Feb. 4, 2010;
U.S. Pat. No. 9,226,981 for PSMA-Binding Agents and Uses Thereof, to Pomper et al., issued Jan. 5, 2016;
U.S. Pat. No. 9,861,713 for PSMA-Binding Agents and Uses Thereof, to Pomper et al., issued Jan. 9, 2018;
U.S. Pat. No. 10,500,292 for PSMA-Binding Agents and Uses Thereof, to Pomper et al., Dec. 10, 2019;
U.S. Patent Application Publication No. 2011/0212025 for TSPO-targeting compounds and uses thereof, Pomper et al., published Sep. 1, 2011.
U.S. Pat. No. 8,778,304 for TSPO-targeting compounds and uses thereof, Pomper et al., issued Jul. 15, 2014;
U.S. Pat. No. 9,233,178 for TSPO-targeting compounds and uses thereof, Pomper et al., issued Jan. 12, 2016;
International PCT Patent Application Publication No. WO2013/138612 for Synthesis and Application of Novel Imaging Agents Conjugated to DPA 713 Analogs for Imaging Inflammation, to Pomper et al., published Sep. 19, 2013.
U.S. Pat. No. 9,498,546 for Synthesis and Application of Novel Imaging Agents Conjugated to DPA 713 Analogs for Imaging Inflammation, to Pomper et al., issued Nov. 22, 2016;
International PCT Patent Application Publication No. WO2018232280 for PSMA Targeted Fluorescent Agents for Image Guided Surgery, to Pomper et al., published Dec. 20, 2018; and
U.S. Published Patent Application No. 2020/0283412 for PSMA Targeted Fluorescent Agents for Image Guided Surgery, to Pomper et al., published Sep. 10, 2020, each of which is incorporated herein by reference in its entirety.

In certain embodiments, the TSPO-targeting compound comprises a compound of formula (I):

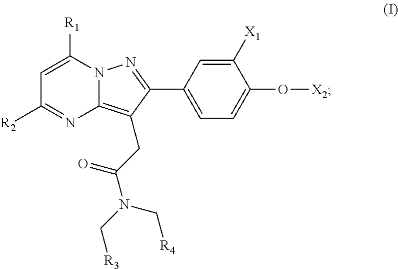

wherein: X₁ can be present or absent and when present is selected from a radioisotope of fluorine, a radioisotope of iodine, a radioisotope of bromine, and a radioisotope of astatine; under the proviso that when X₁ is present, X₂ is H or C₁-C₄ alkyl and when X₁ is absent, X₂ is -L-I, wherein L is a linker and I is an imaging agent; and R₁, R₂, R₃ and R₄ are each independently selected from hydroxyl, C₁ to C₁₀ alkyl, cycloalkyl, aryl, alkylamino, alkylamino, alkenyl, alkynyl, hydroxyalkyl, alkoxyl, dialkylamino thioalkyl, thioalkenyl, thioalkynyl, aryloxy, acyloxy, thioacyl, amido, and sulphonamido; wherein each of alkyl, or aryl moiety may be unsubstituted or substituted with one or more substituents selected from the group consisting of halo, hydroxyl, carboxyl, phosphoryl, phosphonyl, phosphono C₁-C₆ alkyl, carboxy C₁-C₆ alkyl, dicarboxy C₁-C₆ alkyl, dicarboxy halo C₁-C₆ alkyl, sulfonyl, cyano, nitro, alkoxy, alkylthio, acyl, acyloxy, thioacyl, acylthio, aryloxy, amino, alkylamino, dialkylamino, trialkylamino, arylalkylamino, guanidino, aldehydo, ureido, and aminocarbonyl, an amino acid residue, and a substituted amino acid residue; and pharmaceutically acceptable salts thereof.

In particular embodiments, the compound of formula (I) is a compound of formula (Ia):

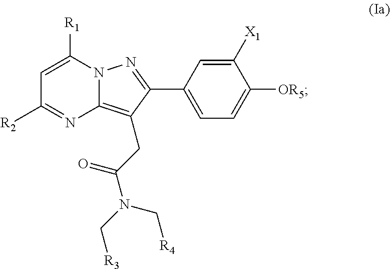

wherein R₅ is H or C₁-C₄ alkyl.

In more particular embodiments, the compound of formula (Ia) is:

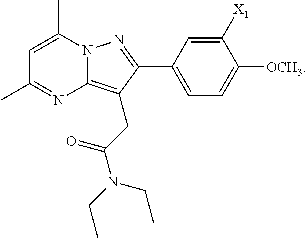

In certain embodiments, X₁ is selected from ¹⁸F, ¹²³I, ¹²⁴I, ¹²⁵I, ¹³¹I, ⁷⁵Br, ⁷⁶Br, ⁷⁷Br, ⁸⁰Br, ^{80m}Br, ⁸²Br, ⁸³Br and ²¹¹At.

In particular embodiments, (a) X₁ is ¹²³I or ¹²⁵I and the imaging is single photon emission computed tomography (SPECT); or (b) X₁ is ¹⁸F or ¹²⁴I and the imaging is positron emission tomography (PET).

In more particular embodiments, the compound of formula (Ia) is:

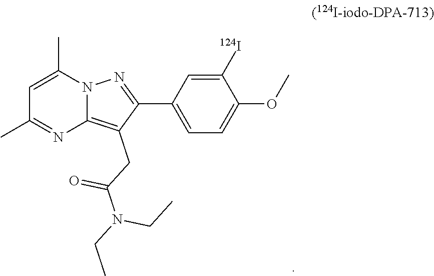

In other embodiments, the compound of formula (I) is a compound of formula (Ib)

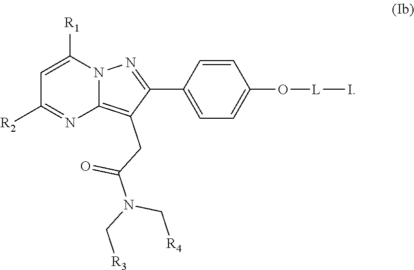

In certain embodiments, the compound of formula (Ib) is:

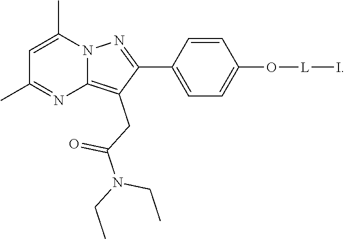

In certain embodiments, L comprises an alkylene linker comprising between 1 to 10 carbon atoms. In certain embodiments, I comprises an imaging agent covalently linked to the linker, L, via an amide linkage.

In certain embodiments, the imaging agent comprises an optical dye. In particular embodiments, the optical dye comprises a fluorescent dye. In more particular embodiments, the fluorescent dye comprises a fluorescent dye that emits in the near infrared spectral region. In even more particular embodiments, the fluorescent dye is selected from a polymethine dye, a coumarin dye, a xanthene dye, and a boron-dipyrromethene (BODIPY) dye.

In certain embodiments, the polymethine dye is selected from a carbocyanine dye, an indocarbocyanine dye, an oxacarbocyanine dye, a thiacarbocyanine dye, and a merocyanine dye. In certain embodiments, the xanthene dye is selected from a fluorescein dye and a coumarin dye.

In particular embodiments, the fluorescent dye is selected from:
BODIPY FL, BODIPY R6G, BODIPY TR, BODIPY TMR, BODIPY 493/503, BODIPY 530/550, BODIPY 558/568, BODIPY 564/570, BODIPY 576/589, BODIPY 581/591, BODIPY 630/650, and BODIPY 650/665;
VivoTag-645, VivoTag-680, VivoTag-S680, VivoTag-S750, VivoTag-800;
Alexa Fluor 488, Alexa Fluor 532, Alexa Fluor 546, Alexa Fluor 555, Alexa Fluor 568, Alexa Fluor 594, Alexa Fluor 647, Alexa Fluor 660, Alexa Fluor 680, Alexa Fluor 700, Alexa Fluor 750, and AlexaFluor790;
Dy677, Dy676, Dy682, Dy752, Dy780;
DyLight 350, DyLight 405, DyLight 488, DyLight 547, DyLight 550, DyLight 594, DyLight 633, DyLight 647, DyLight 650, DyLight 680, DyLight 755, and DyLight 800;
HiLyte Fluor 405, HiLyte Fluor 488, HiLyte Fluor 532, HiLyte Fluor 555, HiLyte™ Fluor 594, HiLyte Fluor 647, HiLyte Fluor 680, HiLyte Fluor 750;
IR800 (Dimethyl{4-[1,5,5-tris(4-dimethylaminophenyl)-2,4-pentadienylidene]-2,5-cyclohexadien-1-ylidene}ammonium perchlorate),
IRDye 650, IRDye 680RD, IRDye 680LT, IRDye 700, IRDye 700DX, IRDye 750, IRDye 800, IRDye 800CW, IRDye 800RS; and
ADS1065A, ADS1075A, ADS775MI, ADS775MP, ADS775PI, ADS775PP, ADS780HO, ADS780WS, ADS785WS, ADS790WS, ADS795WS, ADS798SM, ADS800AT, ADS815EI, ADS830AT, ADS830WS, ADS832WS, ADS845MC, and ADS920MC.

In particular embodiments, the optical dye is selected from:

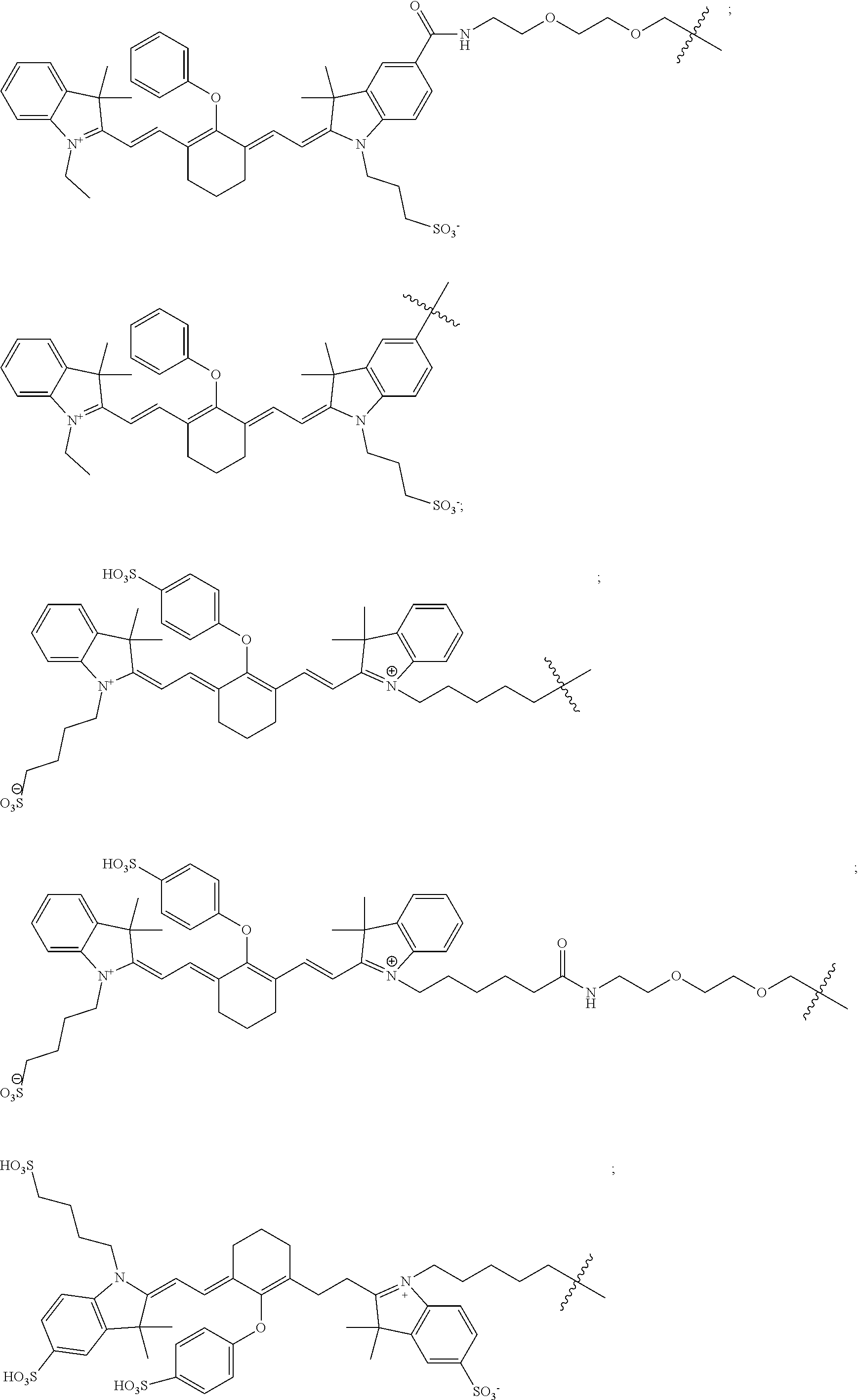

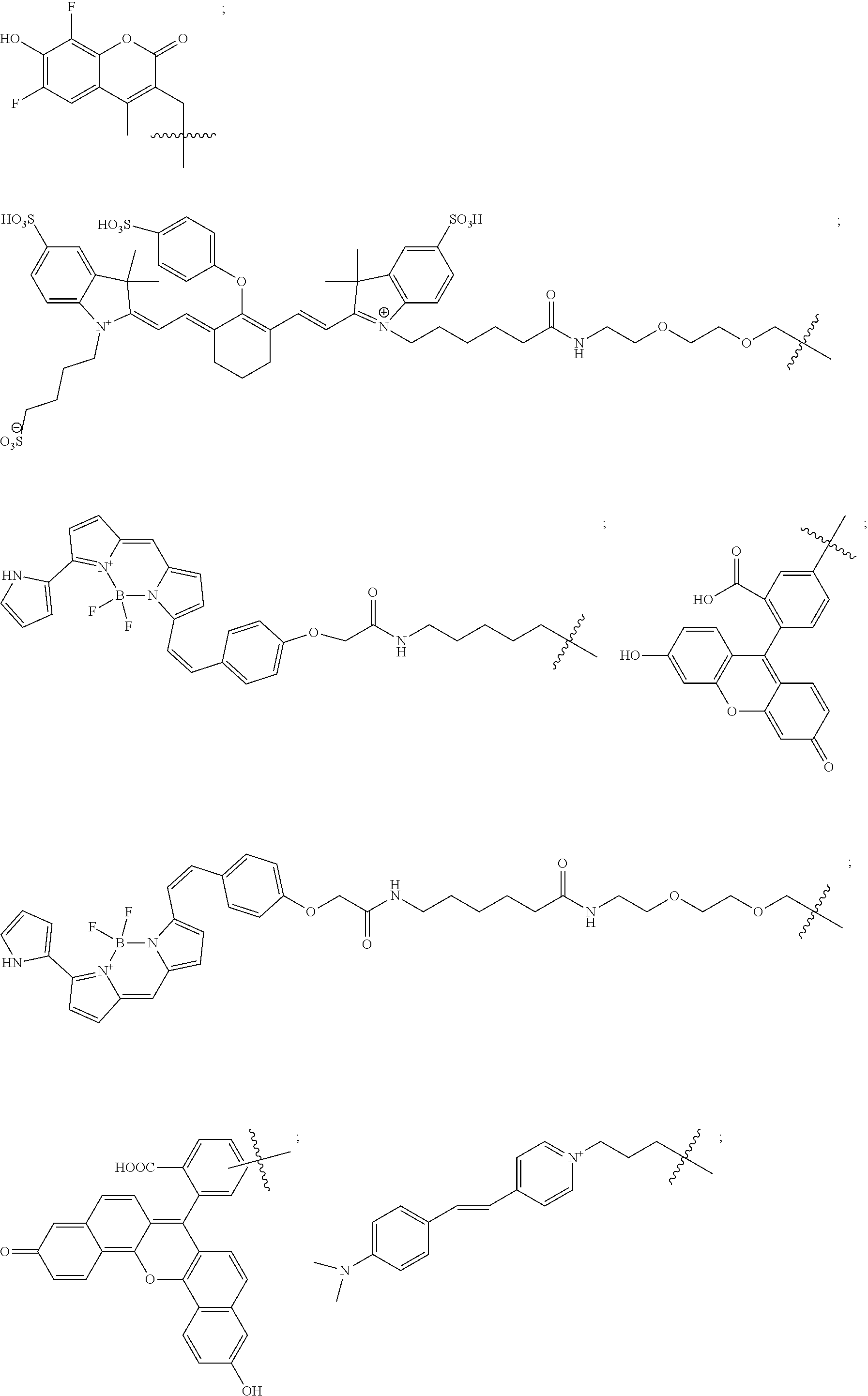

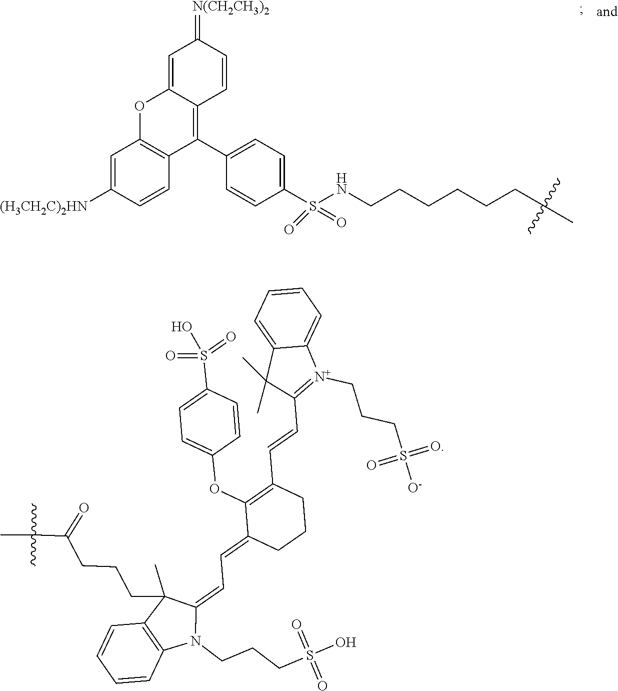

In some embodiments, the PSMA-targeting compound is a compound of formula (II)

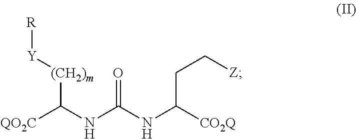

wherein: Z is tetrazole or CO₂Q; each Q is independently selected from hydrogen or a protecting group; and wherein:
(A) m is 0, 1, 2, 3, 4, 5, or 6;
R is a pyridine ring selected from:

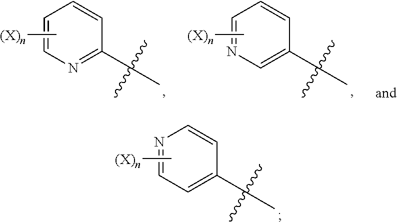

wherein:
X is fluorine, iodine, a radioisotope of fluorine, a radioisotope of iodine, chlorine, bromine, a radioisotope of bromine, a radioisotope of astatine, NO₂, NH₂, N⁺(R²)₃, Sn(R²)₃, Si(R²)₃, Hg(R²), B(OH)₂, —NHNH₂, —NHN═CHR³, —NHNH—CH₂R³;
n is 1, 2, 3, or 4;
Y is O, S, N(R′), C(O), NR′C(O), C(O)N(R′), OC(O), C(O)O, NR′C(O)NR′, NR′C(S)NR′, NR′S(O)₂, S(CH₂)ₚ, NR′(CH₂)ₚ, O(CH₂)ₚ, OC(O)CHR⁸NHC(O), NHC(O)CHR⁸NHC(O), or a covalent bond; wherein p is 1, 2, or 3, R′ is H or C₁-C₆ alkyl, and R⁸ is hydrogen, alkyl, aryl or heteroaryl, each of which may be substituted;
R² is C₁-C₆ alkyl; and
R³ is alkyl, alkenyl, alkynyl, aryl, or heteroaryl each of which is substituted by fluorine, iodine, a radioisotope of fluorine, a radioisotope of iodine, chlorine, bromine, a radioisotope of bromine, or a radioisotope of astatine, NO₂, NH₂, N⁺(R²)₃, Sn(R²)₃, Si(R²)₃, Hg(R²), or B(OH)₂; or
(B) m is 0, 1, 2, 3, 4, 5, or 6;
Y is O, S, N(R′), C(O), NR′C(O), C(O)N(R′), OC(O), C(O)O, NR′C(O)NR′, NR′C(S)NR′, NR′S(O)₂, S(CH₂)ₚ, NR′(CH₂)ₚ, O(CH₂)ₚ, OC(O)CHR⁸NHC(O), NHC(O)CHR⁸NHC(O), or a covalent bond; wherein p is 1, 2, or 3, R′ is H or C₁-C₆ alkyl, and R⁸ is hydrogen alkyl, aryl or heteroaryl, each of which may be substituted;
R is:

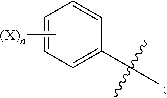

wherein:
X′ is selected from the group consisting of NHNH₂, —NHN═CHR³, and —NHNH—CH₂R³; wherein R³ is alkyl, alkenyl, alkynyl, aryl, or heteroaryl each of which is substituted by fluorine, iodine, a radioisotope of fluorine, a radioisotope of iodine, bromine, a radioisotope of bromine, or a radioisotope of astatine; NO₂, NH₂, N⁺(R²)₃, Sn(R²)₃, Si(R²)₃, Hg(R²), or B(OH)₂;
R² is C₁-C₆ alkyl;
n is 1, 2, 3, 4, or 5; or
(C) m is 4;
Y is NR′; and
R is:

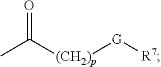

wherein G is 0, NR′ or a covalent bond;
R is H or C₁-C₆ alkyl; p is 1, 2, 3, or 4, and
R⁷ is selected from the group consisting of NH₂, N═CHR³, NH—CH₂R³, wherein R³ is alkyl, alkenyl, alkynyl, aryl, or heteroaryl each of which is substituted by fluorine, iodine, a radioisotope of fluorine, a radioisotope of iodine, chlorine, bromine, a radioisotope of bromine, or a radioisotope of astatine, NO₂, NH₂, N⁺(R²)₃, Sn(R²)₃, Si(R²)₃, Hg(R²), B(OH)₂; and R² is C₁-C₆ alkyl.

In certain embodiments, the compound of formula (II) has the structure:

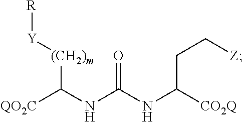

wherein m is 0, 1, 2, 3, 4, 5, or 6;
R is a pyridine ring selected from the group consisting of:

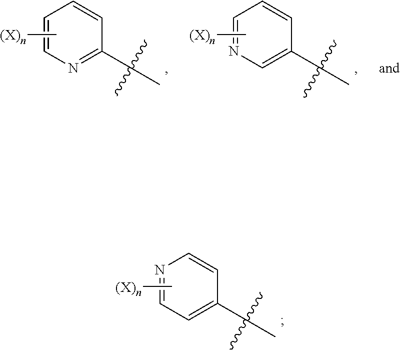

wherein:
X is fluorine, iodine, a radioisotope of fluorine, a radioisotope of iodine, chlorine, bromine, a radioisotope of bromine, a radioisotope of astatine, NO₂, NH₂, N⁺(R²)₃, Sn(R²)₃, Si(R²)₃, Hg(R²), B(OH)₂, —NHNH₂, —NHN═CHR³, and —NHNH—CH₂R³;
each Q is independently selected from hydrogen or a protecting group;
Y is O, S, N(R′), C(O), NR′C(O), C(O)N(R′), OC(O), C(O)O, NR′C(O)NR′, NR′C(S)NR′, NR′S(O)₂, S(CH₂)ₚ, NR′(CH₂)ₚ, O(CH₂)ₚ, OC(O)CHR⁸NHC(O), NHC(O)CHR⁸NHC(O), or a covalent bond; wherein p is 1, 2, or 3, R′ is H or C₁-C₆ alkyl, and R⁸ is hydrogen, alkyl, aryl or heteroaryl, each of which may be substituted;
Z is tetrazole or CO₂Q;
R² is C₁-C₆ alkyl; and R³ is alkyl, alkenyl, alkynyl, aryl, or heteroaryl, each of which is substituted by fluorine, iodine, a radioisotope of fluorine, a radioisotope of iodine, chlorine, bromine, a radioisotope of bromine, or a radioisotope of astatine; NO₂, NH₂, N⁺(R²)₃, Sn(R²)₃, Si(R²)₃, Hg(R²), or B(OH)₂.

In certain embodiments, the compound of formula (II) has the structure:

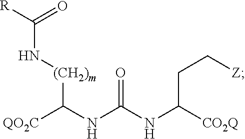

wherein m is not 0.

In certain embodiments, the compound of formula (II) has the structure:

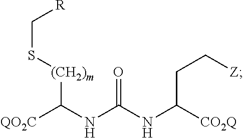

wherein m is not 0.

In certain embodiments, for the compound of formula (II):
m is 0, 1, 2, 3, 4, 5, or 6;
Y is O, S, N(R′), C(O), NR′C(O), C(O)N(R′), OC(O), C(O)O, NR′C(O)NR′, NR′C(S)NR′, NR′S(O)₂, S(CH₂)ₚ, NR′(CH₂)ₚ, O(CH₂)ₚ, OC(O)CHR⁸NHC(O), NHC(O)CHR⁸NHC(O), or a covalent bond; wherein p is 1, 2, or 3, R′ is H or C₁-C₆ alkyl, and R⁸ is hydrogen, alkyl, aryl or heteroaryl, each of which may be substituted;
R is:

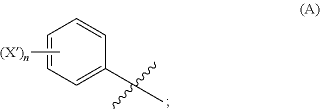

wherein:
X′ is selected from the group consisting of NHNH₂, —NHN═CHR³, —NHNH—CH₂R³; wherein R³ is alkyl, alkenyl, alkynyl, aryl, or heteroaryl, each of which is substituted by fluorine, iodine, a radioisotope of fluorine, a radioisotope of iodine, bromine, a radioisotope of bromine, or a radioisotope of astatine; NO₂, NH₂, N⁺(R²)₃, Sn(R²)₃, Si(R²)₃, Hg(R²), and B(OH)₂, where R² is C₁-C₆ alkyl;
n is 1, 2, 3, 4, or 5.

In certain embodiments of the compound of formula (II), X or X′ is fluorine, iodine, or a radioisotope of fluorine or iodine, bromine, a radioisotope of bromine, or a radioisotope of astatine. In particular embodiments of the compound of formula (II), X or X′ is selected from ¹⁸F, ¹²³I, ¹²⁴I, ¹²⁵I, ¹³¹I, ⁷⁵Br, ⁷⁶Br, ⁷⁷Br, ⁸⁰Br, ^{80m}Br, ⁸²Br, ⁸³Br and ²¹¹At. In more particular embodiments of the compound of formula (II), X or X′ is ¹⁸F.

In particular embodiments, the compound of formula (II) is selected from:

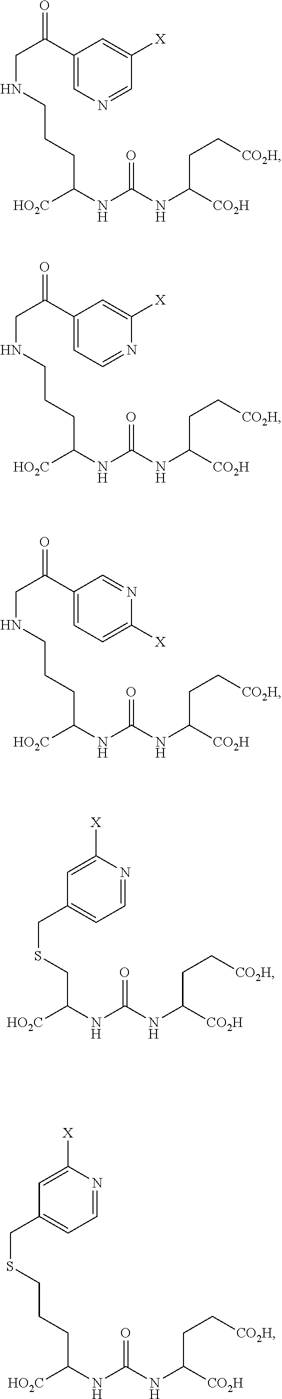

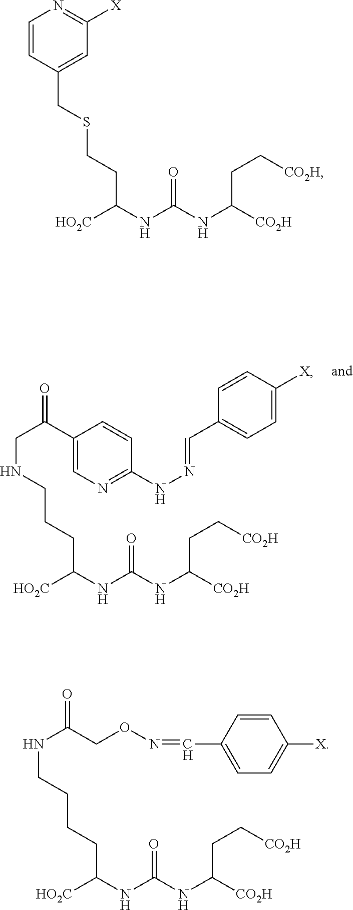

In more particular embodiments, the compound of formula (II) is:

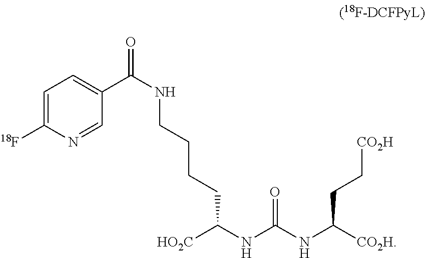 [SMILES: O=C(O)CC[C@H](NC(=O)N[C@@H](CCCCNC(=O)c1ccc([18F])nc1)C(=O)O)C(=O)O]

In other embodiments, the PSMA-targeting compound comprises a compound of formula (III):

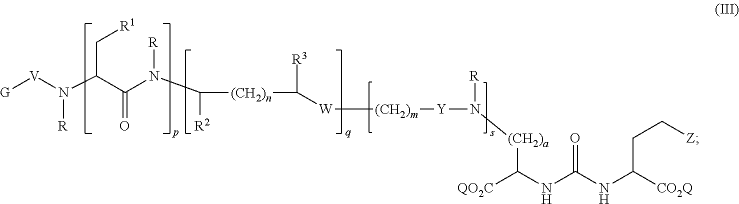

wherein:
q and s are each independently 0 or 1;
p is 0, 1, 2, or 3;
a is 1, 2, 3, or 4;
m is 1, 2, 3, 4, 5, or 6;
n is 1, 2, 3, 4, 5 or 6;
Z is tetrazole or CO₂Q;
each Q is independently selected from hydrogen or a protecting group;
V can be present or absent and when is present is selected from —C(O)—, —NRC(O)—, and —NRC(S)—;
W is selected from —NRC(O)—, —NRC(O)NR—, NRC(S)NR—, —NRC(O)O—, —OC(O)NR—, —OC(O)—, —C(O)NR—, or —C(O)O—;
Y is selected from —C(O)—, —NRC(O)—, —NRC(S)—, and —OC(O);
each R is independently H or C₁-C₄ alkyl;
each R¹ is independently H, C₁-C₆ alkyl, C₂-C₁₂ aryl, or C₄-C₁₆ alkylaryl, whereas when p is 2 or 3, each R¹ may be the same or different;
R² and R³ are independently H, CO₂H, or CO₂R⁴, where R⁴ is a C₁-C₆ alkyl, C₂-C₁₂ aryl, or C₄-C₁₆ alkylaryl, wherein when one of R² and R³ is CO₂H or CO₂R⁴, the other is H; and
G is a metal chelating moiety optionally including a chelated metal or a dye moiety that emits in the visible or near infrared spectrum, wherein G can include any additional atoms or linkers necessary to attach the metal chelating moiety or dye moiety to the rest of the compound. For instance linking groups having alkyl, aryl, combination of alkyl and aryl, or alkyl and aryl groups having heteroatoms may be present in the chelating or dye moiety, so long as the linker does not interfere with the binding of the chelating moiety or the optical properties of the dye. In some embodiments, the linking group includes a poly(ethylene glycol) linker. The chelating moiety or dye moiety can include activated groups used to react with protein sidechains or other compounds. For example, such agents include, but are not limited to, N-hydroxysuccinimide (NHS), N-hydroxysulfosuccinimide (sulfo-NHS), anhydride, maleimide, N-benzyl, 4-isothiocyanatobenzyl (p-NCS-Bz), NH₂-MPAA, propargyl, TA, N-(2-aminoethyl)ethanamide, NH₂—PEG4, and hydrophilic dPEG spacer bound to a tetrafluorophenyl (TFP) ester. These agents can be bound to or form part of the linker which is bound to the chelating or dye moiety.

In certain embodiments, the compound of formula (III) has the following structure:

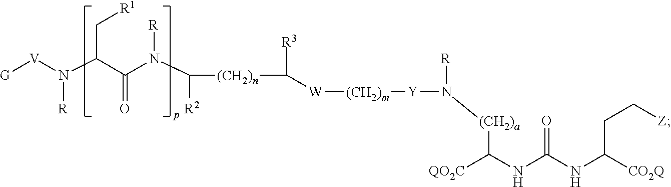

wherein:
Z is tetrazole or CO₂Q;
each Q is independently selected from hydrogen or a protecting group;
a is 1, 2, 3, or 4;
m is 1, 2, 3, 4, 5, or 6;
n is 1, 2, 3, 4, 5 or 6;
p is 0, 1, 2, or 3;
each R is independently H or C₁-C₄ alkyl;
V is selected from —C(O)—, —NRC(O)—, and —NRC(S)—;
W is selected from —NRC(O)—, —NRC(O)NR—, NRC(S)NR—, —NRC(O)O—, —OC(O)NR—, —OC(O)—, —C(O)NR—, and —C(O)O—;
Y is selected from —C(O)—, —NRC(O)—, —NRC(S)—, and —OC(O)—;
R¹ is H, C₁-C₆ alkyl, C₂-C₁₂ aryl, or C₄-C₁₆ alkylaryl, whereas when p is 2 or 3, each R¹ may be the same or different;
R² and R³ are independently H, CO₂H, or CO₂R⁴, where R⁴ is a C₁-C₆ alkyl, C₂-C₁₂ aryl, or C₄-C₁₆ alkylaryl, wherein when one of R² and R³ is CO₂H or CO₂R⁴, the other is H; and
G a metal chelating moiety optionally including a chelated metal or a dye moiety that emits in the visible or near infrared spectrum.

In certain embodiments, the compound of formula (III) has the following structure:

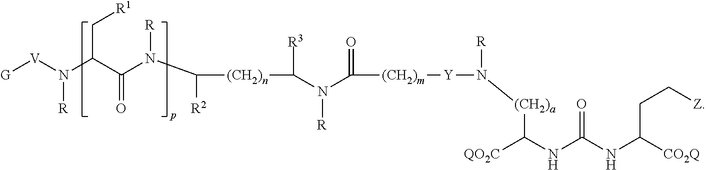

In certain embodiments, the compound of formula (III) has the following structure:

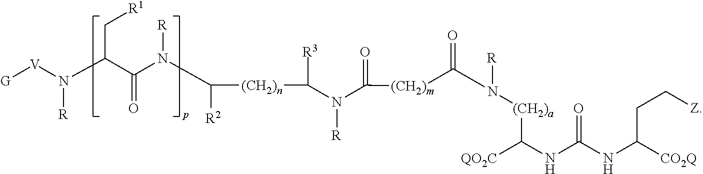

In certain embodiments, the compound of formula (III) has the following formula:

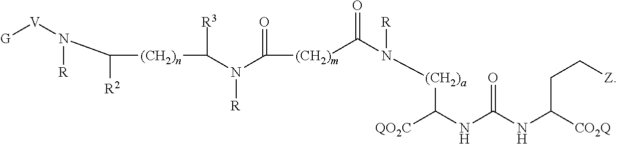

In certain embodiments, the compound of formula (III), R¹ is phenyl. In certain embodiments, for the compound of formula (III), R⁴ is benzyl.

In certain embodiments, for the compound of formula (III): (a) R² is H, and R³ is H; (b) R³ is CO₂H and R² is H; (c) R² is CO₂H and R³ is H; (d) R² is CO₂R⁴ and R³ is H; or (e) R³ is CO₂R⁴, and R² is H.

In some embodiments, G is an optical dye. In particular embodiments, the optical dye comprises a fluorescent dye. In more particular embodiments, the fluorescent dye comprises a fluorescent dye that emits in the near infrared spectral region. In even more particular embodiments, the fluorescent dye is selected from a polymethine dye, a coumarin dye, a xanthene dye, and a boron-dipyrromethene (BODIPY) dye.

In certain embodiments, the polymethine dye is selected from a carbocyanine dye, an indocarbocyanine dye, an oxacarbocyanine dye, a thiacarbocyanine dye, and a merocyanine dye. In certain embodiments, the xanthene dye is selected from a fluorescein dye and a coumarin dye.

In particular embodiments, the fluorescent dye is selected from:
BODIPY FL, BODIPY R⁶G, BODIPY TR, BODIPY TMR, BODIPY 493/503, BODIPY 530/550, BODIPY 558/568, BODIPY 564/570, BODIPY 576/589, BODIPY 581/591, BODIPY 630/650, and BODIPY 650/665;
VivoTag-645, VivoTag-680, VivoTag-S680, VivoTag-S750, VivoTag-800;
Alexa Fluor 488, Alexa Fluor 532, Alexa Fluor 546, Alexa Fluor 555, Alexa Fluor 568, Alexa Fluor 594, Alexa Fluor 647, Alexa Fluor 660, Alexa Fluor 680, Alexa Fluor 700, Alexa Fluor 750, and AlexaFluor790;
Dy677, Dy676, Dy682, Dy752, Dy780;
DyLight 350, DyLight 405, DyLight 488, DyLight 547, DyLight 550, DyLight 594, DyLight 633, DyLight 647, DyLight 650, DyLight 680, DyLight 755, and DyLight 800;
HiLyte Fluor 405, HiLyte Fluor 488, HiLyte Fluor 532, HiLyte Fluor 555, HiLyte™ Fluor 594, HiLyte Fluor 647, HiLyte Fluor 680, HiLyte Fluor 750;
IR800 (Dimethyl{4-[1,5,5-tris(4-dimethylaminophenyl)-2,4-pentadienylidene]-2,5-cyclohexadien-1-ylidene}ammonium perchlorate),
IRDye 650, IRDye 680RD, IRDye 680LT, IRDye 700, IRDye 700DX, IRDye 750, IRDye 800, IRDye 800CW, IRDye 800RS; and
ADS1065A, ADS1075A, ADS775MI, ADS775MP, ADS775PI, ADS775PP, ADS780HO, ADS780WS, ADS785WS, ADS790WS, ADS795WS, ADS798SM, ADS800AT, ADS815EI, ADS830AT, ADS830WS, ADS832WS, ADS845MC, and ADS920MC.

In particular embodiments, the optical dye is selected from:

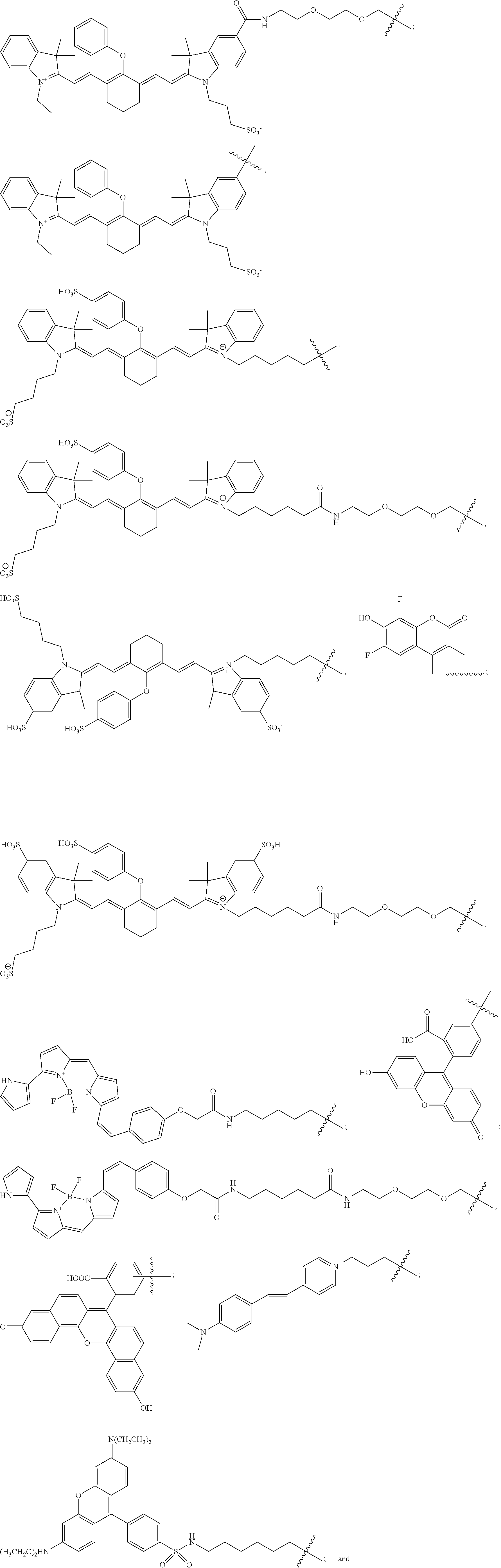

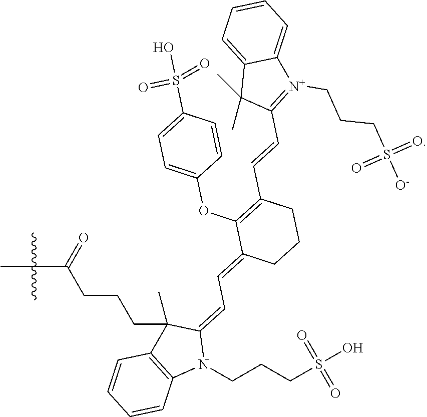 [SMILES: *C(=O)CCCC1(C)/C(=C\C=C2/CCCC(/C=C/C3=[N+](CCCS(=O)(=O)[O-])c4ccccc4C3(C)C)=C2Oc2ccc(S(=O)(=O)O)cc2)N(CCCS(=O)(=O)O)c2ccccc21]

In particular embodiments of the compound of formula (III), G is an optical dye and the compound of formula (III) is selected from:

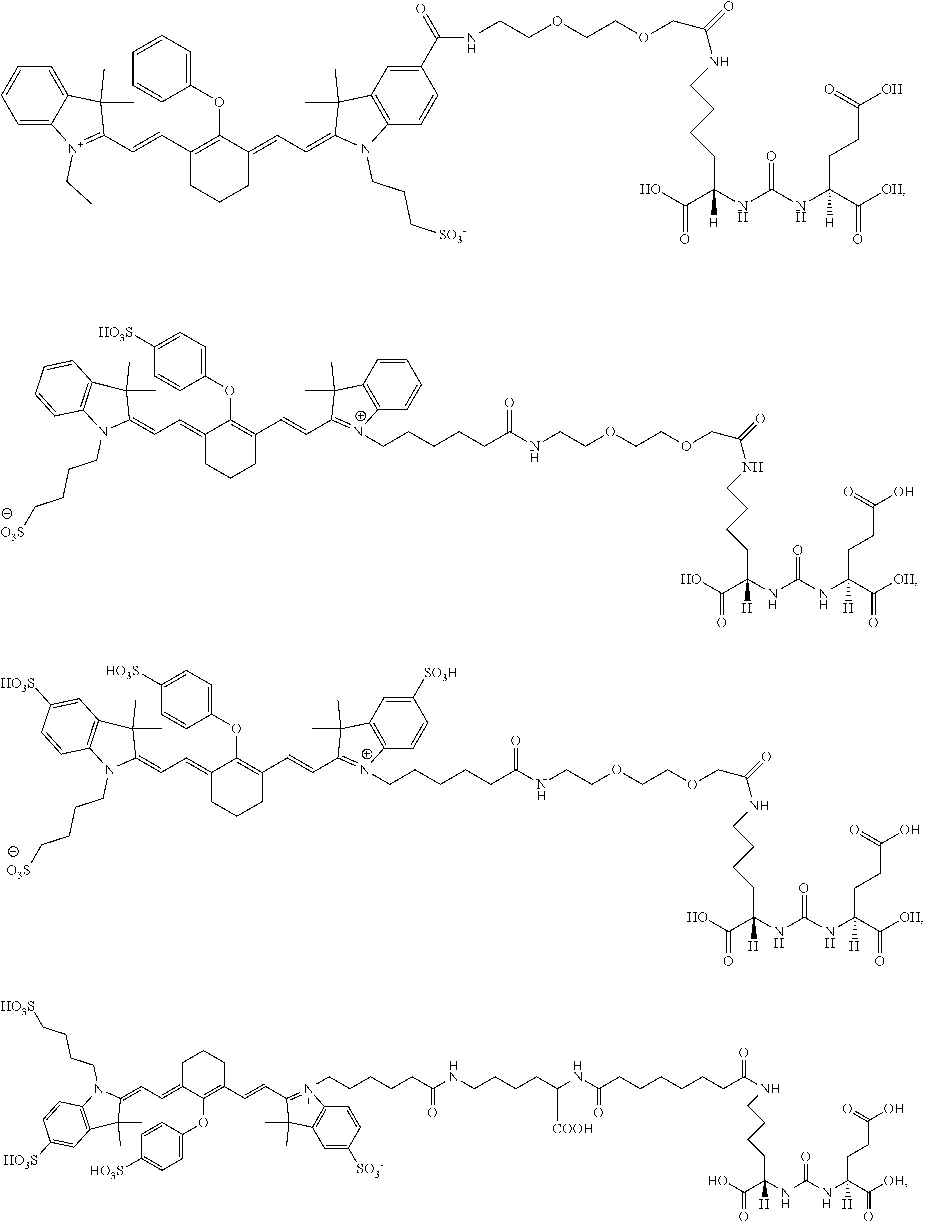

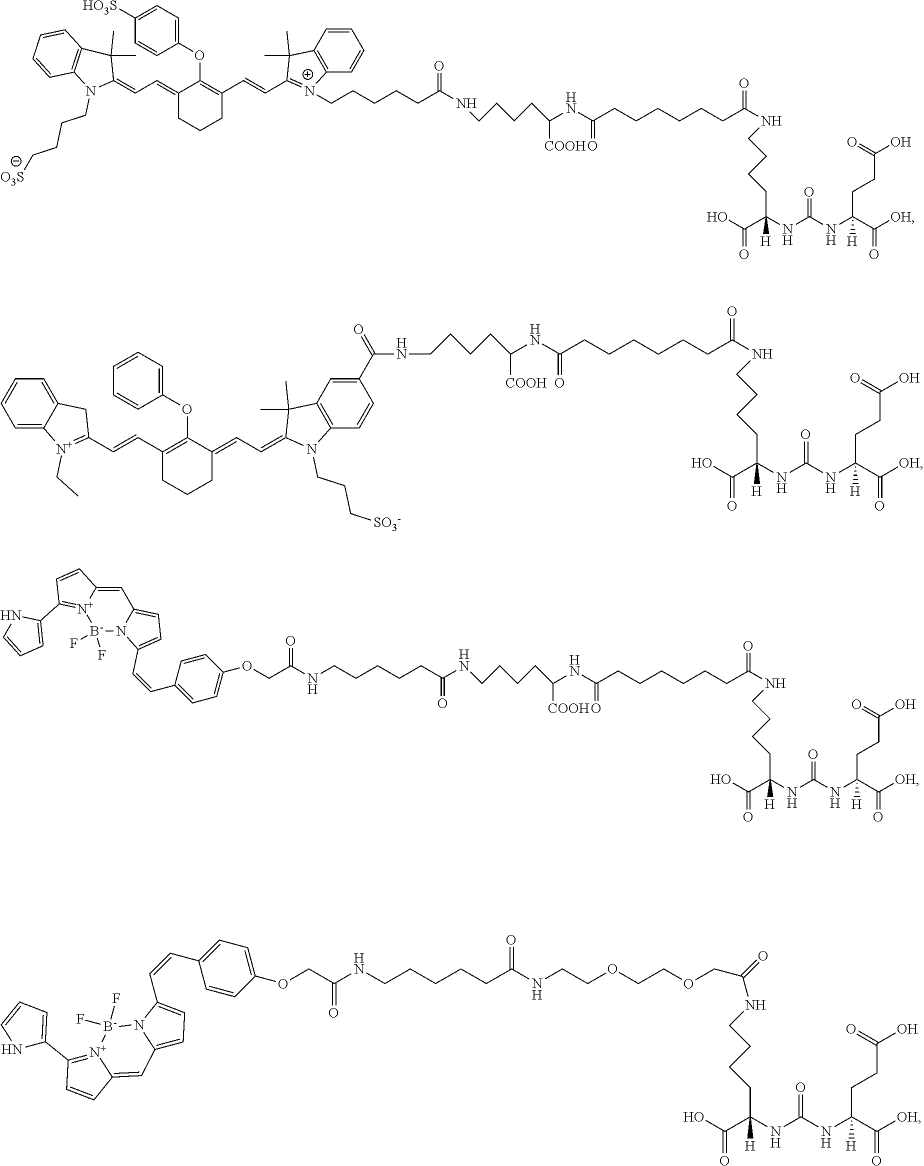

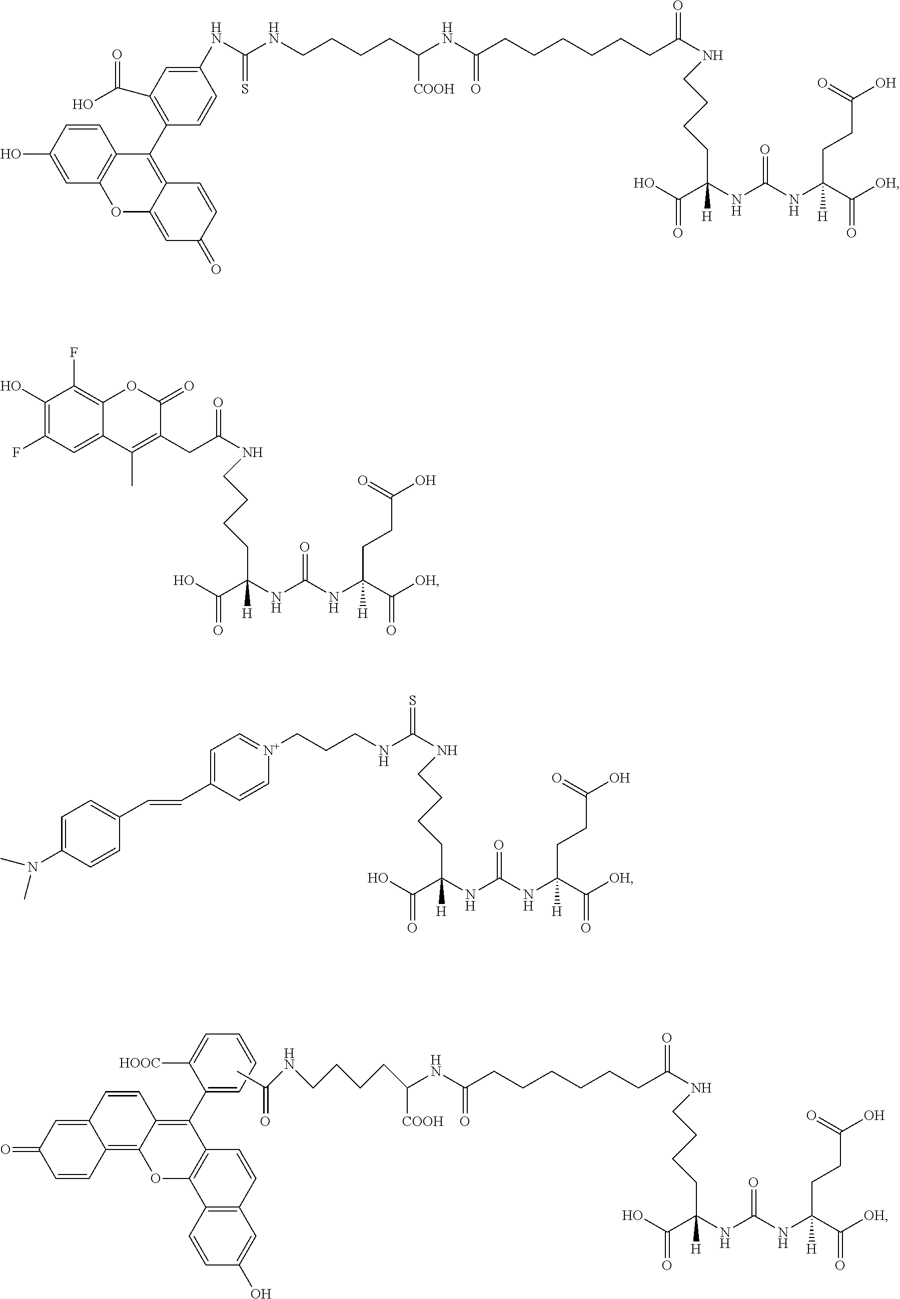

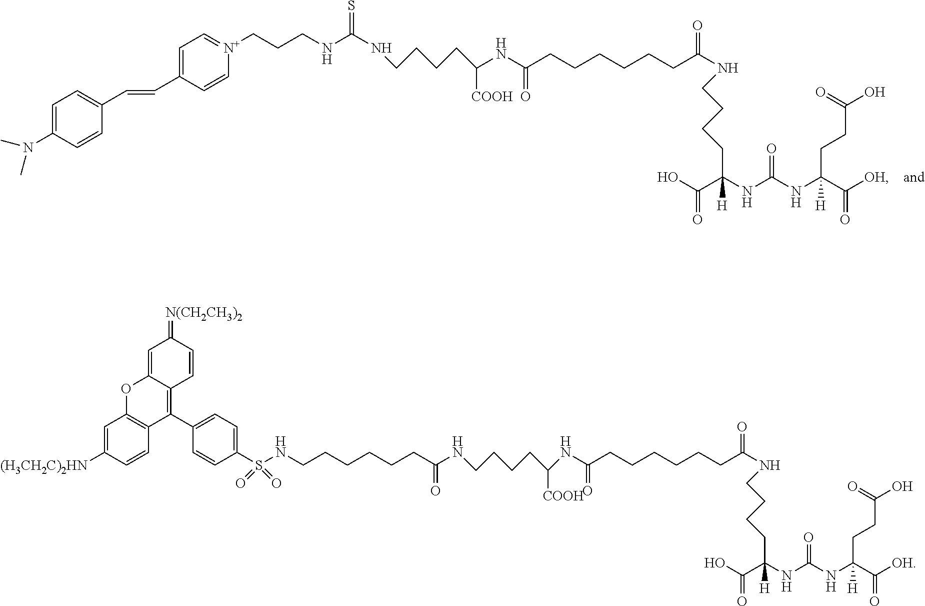

In more particular embodiments, the compound of formula (III) is:

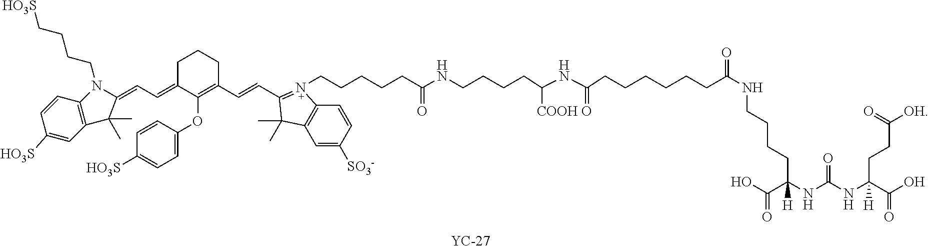

In other embodiments, for the compound of formula (III), the metal chelating moiety is selected from DOTAGA (1,4,7,10-tetraazacyclododececane,1-(glutaric acid)-4,7,10-triacetic acid), DOTA (1,4,7,10-tetraazacyclododecane-1,4,7,10-tetraacetic acid), DOTASA (1,4,7,10-tetraazacyclododecane-1-(2-succinic acid)-4,7,10-triacetic acid), CB-DO2A (10-bis(carboxymethyl)-1,4,7,10-tetraazabicyclo[5.5.2]tetradecane), DEPA (7-[2-(Bis-carboxymethylamino)-ethyl]-4,10-bis-carboxymethyl-1,4,7,10-tetraaza-cyclododec-1-yl-acetic acid)), 3p-C-DEPA (2-[(carboxymethyl)][5-(4-nitrophenyl-1-[4,7,10-tris(carboxymethyl)-1,4,7,10-tetraazacyclododecan-1-yl]pentan-2-yl)amino]acetic acid)), TCMC (2-(4-isothiocyanotobenzyl)-1,4,7,10-tetraaza-1,4,7,10-tetra-(2-carbamonyl methyl)-cyclododecane), oxo-DO3A (1-oxa-4,7,10-triazacyclododecane-5-S-(4-isothiocyanatobenzyl)-4,7,10-triacetic acid), p-NH₂-Bn-Oxo-DO3A (1-Oxa-4,7,10-tetraazacyclododecane-5-S-(4-aminobenzyl)-4,7,10-triacetic acid), TE2A ((1,8-N,N′-bis-(carboxymethyl)-1,4,8,11-tetraazacyclotetradecane), MM-TE2A, DM-TE2A, CB-TE2A (4,11-bis(carboxymethyl)-1,4,8,11-tetraazabicyclo[6.6.2]hexadecane), CB-TE1A1P (4,8,11-tetraazacyclotetradecane-1-(methanephosphonic acid)-8-(methanecarboxylic acid)), CB-TE2P (1,4,8,11-tetraazacyclotetradecane-1,8-bis(methanephosphonic acid), TETA (1,4,8,11-tetraazacyclotetradecane-1,4,8,11-tetraacetic acid), NOTA (1,4,7-triazacyclononane-N,N′,N″-triacetic acid), NODA (1,4,7-triazacyclononane-1,4-diacetate); NODAGA (1,4,7-triazacyclononane,1-glutaric acid-4,7-acetic acid), (NOTAGA) 1,4,7-triazonane-1,4-diyl)diacetic acid, DFO (Deferoxamine), NETA ([4-[2-(bis-carboxymethylamino)-ethyl]-7-carboxymethl-[1,4,7]triazonan-1-yl}-acetic acid), TACN-TM (N,N′,N″, tris(2-mercaptoethyl)-1,4,7-triazacyclononane), Diamsar (1,8-Diamino-3,6,10,13,16,19-hexaazabicyclo(6,6,6)eicosane, 3,6,10,13,16,19-Hexaazabicyclo[6.6.6]eicosane-1,8-diamine), Sarar (1-N-(4-aminobenzyl)-3,6,10,13,16,19-hexaazabicyclo[6.6.6]eicosane-1,8-diamine), AmBaSar (4-((8-amino-3,6,10,13,16,19-hexaazabicyclo[6.6.6]icosane-1-ylamino) methyl) benzoic acid), and BaBaSar.

In particular embodiments, the compound of formula (III), the chelating agent is selected from:

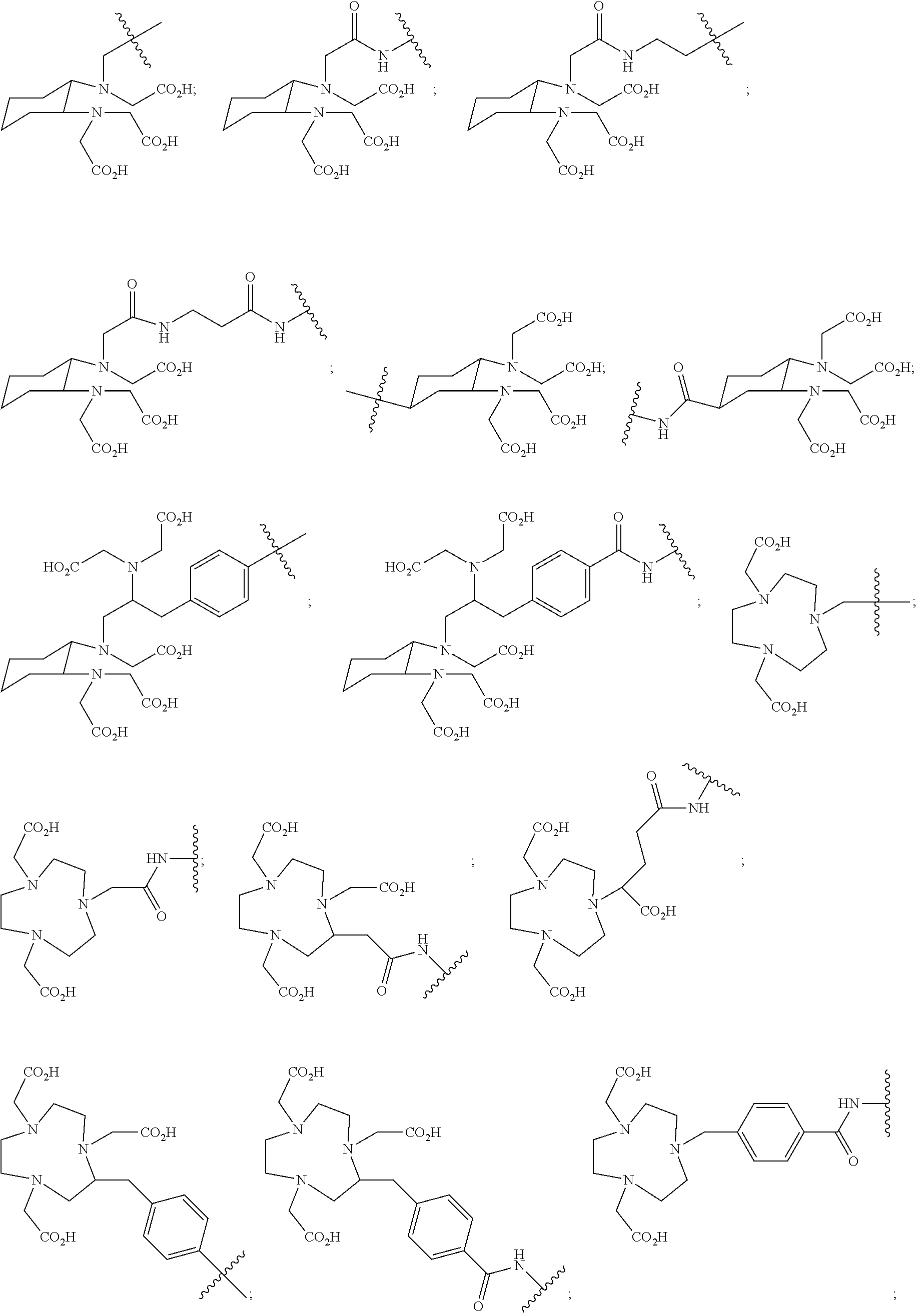

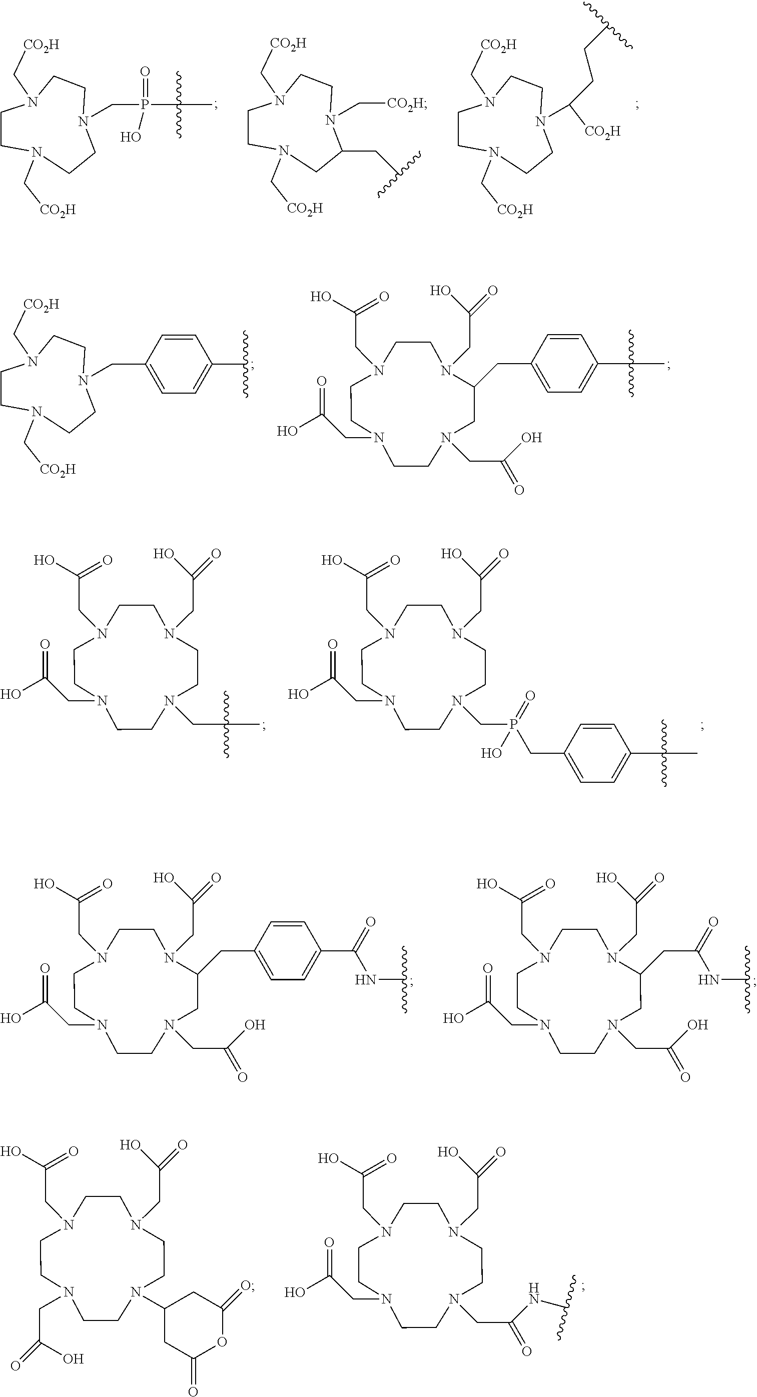

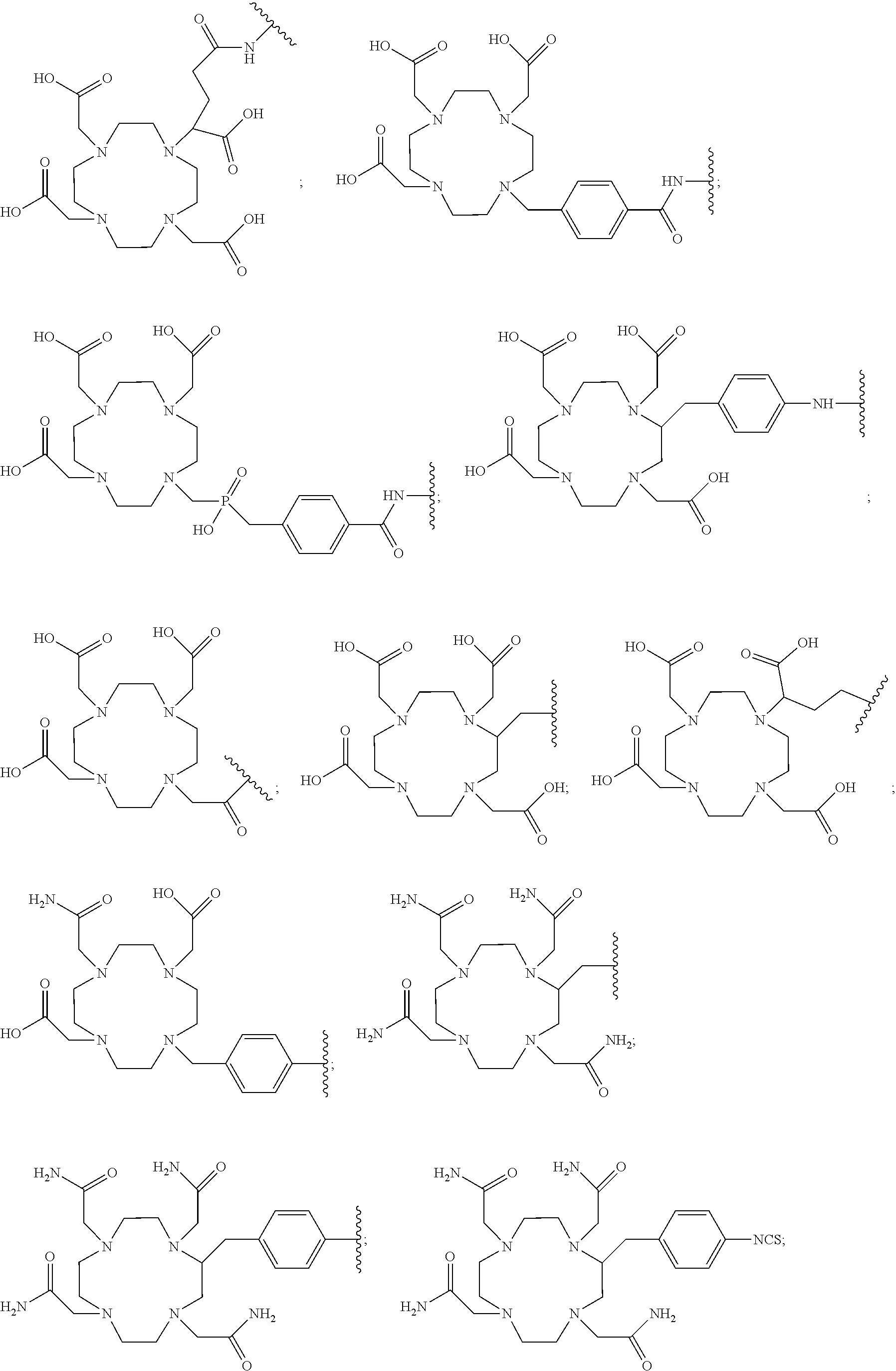

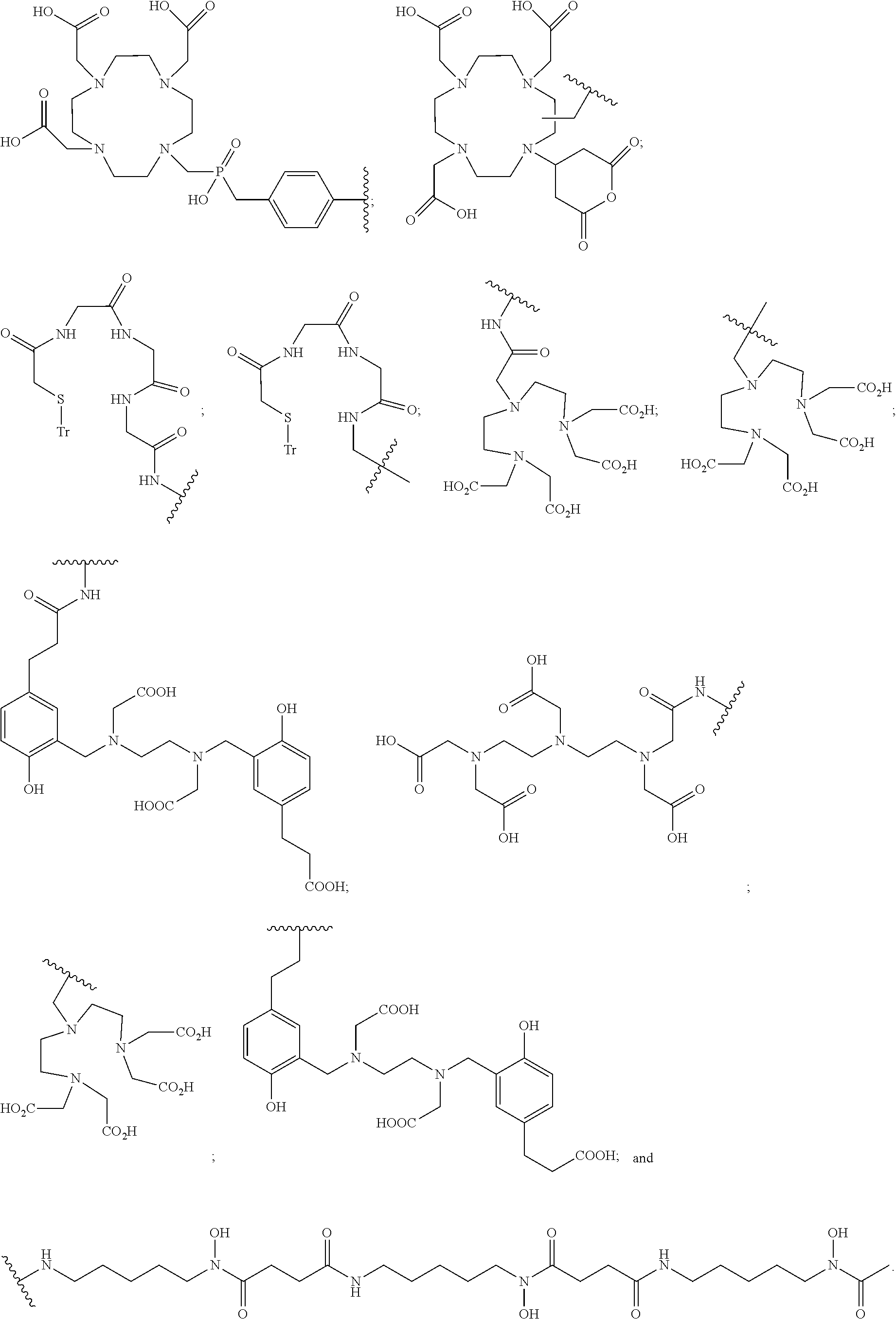

In more particular embodiments, the compound of formula (III) is selected from:

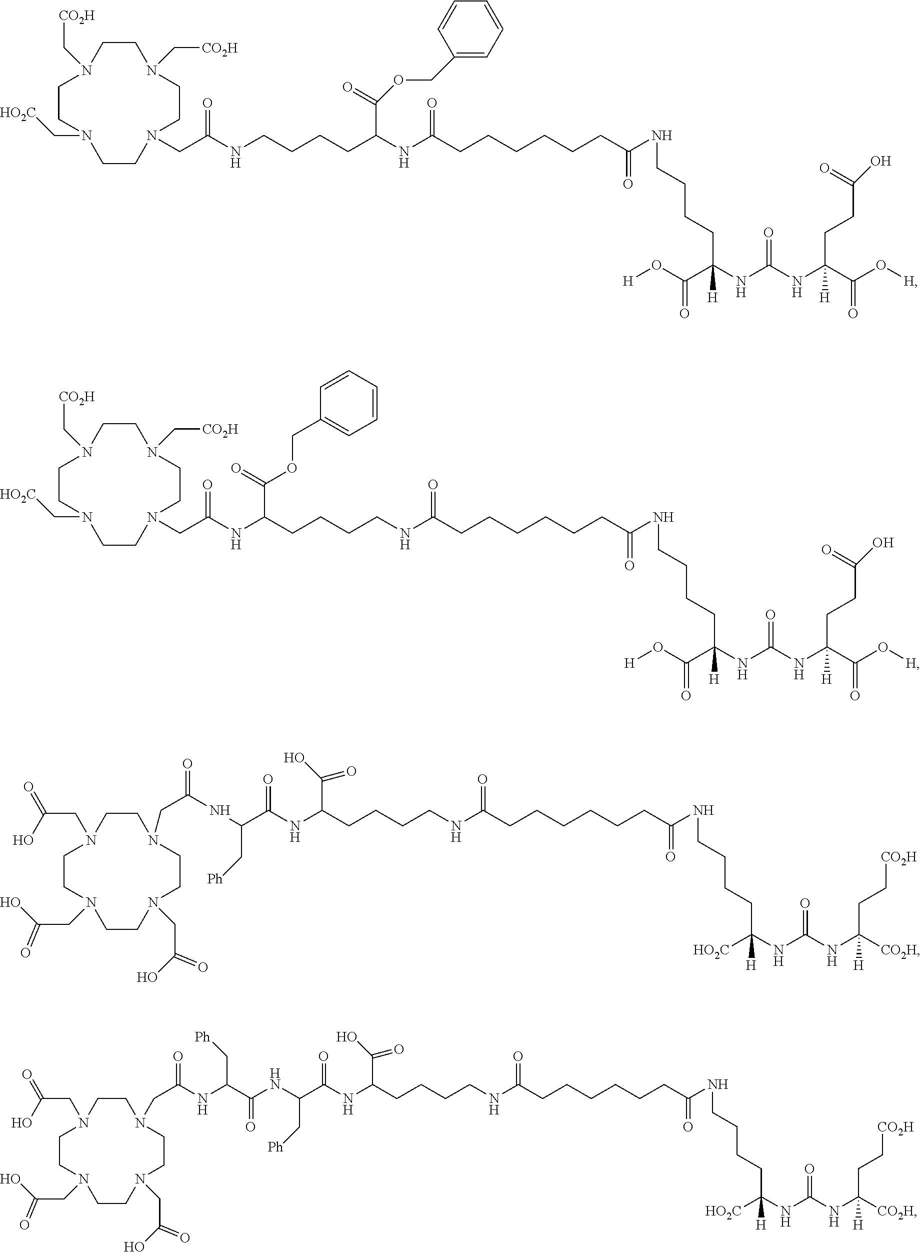

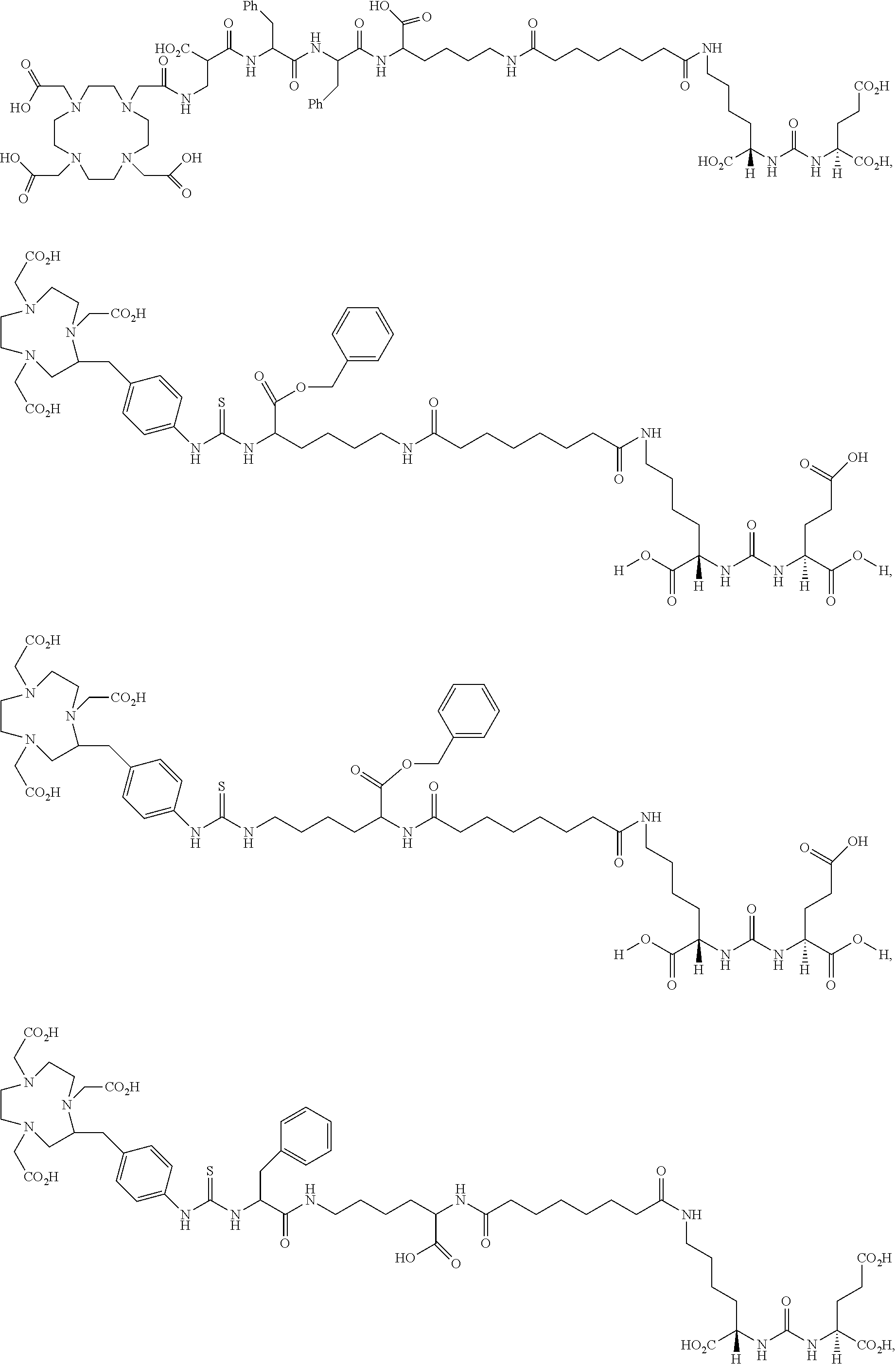

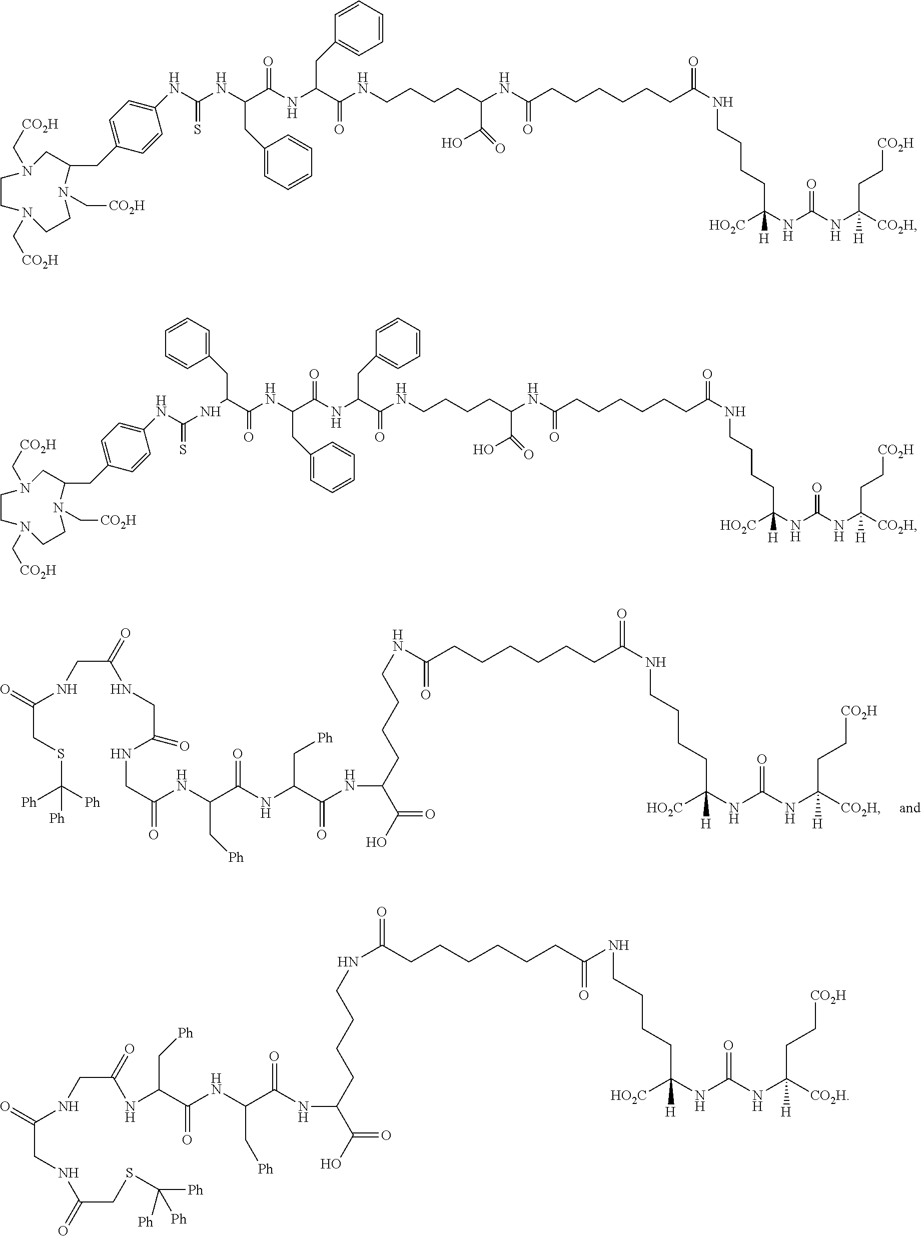

In certain embodiments, for the compound of formula (III), the chelated metal is selected from ⁶⁰Cu, ⁶²Cu, ⁶⁴Cu, ⁶⁷Cu, ⁵⁵Co, ⁵⁷Co, ²⁰³Pb, ²¹²Pb, ²²⁵Ac, ¹⁷⁷Lu, ^{99m}Tc ⁶⁷Ga, ⁶⁸Ga, ¹⁴⁹Tb, ⁸⁶Y, ⁹⁰Y. ¹¹¹In, ¹⁸⁶Re, ¹⁸⁸Re, ¹⁵³Sm, ⁸⁹Zr, ²¹³Bi, ²¹²Bi, ²¹²Pb, ⁶⁷Ga, ⁴⁷Sc, ¹⁶⁶Dy, Al¹⁸F, ¹⁶⁶Ho, and ¹⁷⁷Lu.

In other embodiments, the PSMA-targeting compound is a compound of formula (IV):

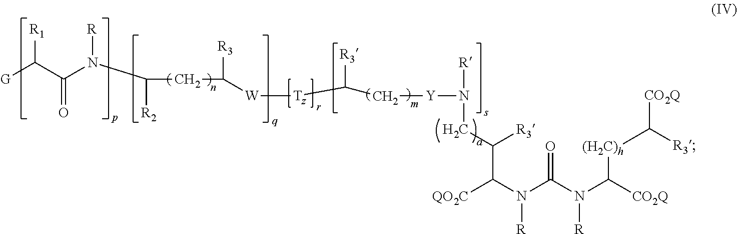

wherein:
each Q is independently hydrogen, a metal ion, a negative charge, or a protecting group;
a, h, m, and n are each independently an integer selected from the group consisting of 1, 2, 3, 4, 5, and 6;
s is 0 or 1;
r is 0 or 1;
q is 0 or 1;
p is an integer selected from the group consisting of 0, 1, 2, and 3, and when p is 2 or 3, each R and Ri can be the same or different;
each R, R′, and R₁ is independently hydrogen, C₁-C₆ substituted or unsubstituted alkyl, C₆-C₁₂ substituted or unsubstituted aryl, C₆-C₁₂ substituted or unsubstituted heteroaryl, or C₆-C₁₆ alkyaryl;
R₂, R₃, and R₃′ are each independently hydrogen, C₁-C₄ substituted or unsubstituted alkyl, —CO₂H,-CO₂Q, or —CO₂R⁴, wherein R₄ is a C₁-C₆ substituted or unsubstituted alkyl, C₆-C₁₂ substituted or unsubstituted aryl, C₆-C₁₂ substituted or unsubstituted heteroaryl, or C₆-C₁₆ alkyaryl, wherein if one of R₂ and R₃ is —CO₂H, or —CO₂R⁴, then the other is H;
Tz is a triazole containing moiety selected from the group consisting of:

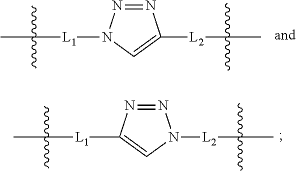

Wherein: L₁ is

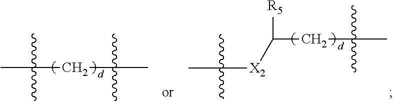

and L₂ is

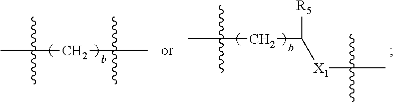

wherein:
X₁ is —NRC(O)—, —NRC(O)NR—, —NRC(S)NR, or —NRC(O)O—;
X₂ is —C(O)NR—, —NRC(O)NR—, —NRC(S)NR—, or —OC(O)NR—;
R₅ is H, —CO₂H, or —CO₂R⁶, wherein R₆ is C₁-C₆ alkyl, C₆-C₁₂ aryl, or C₆-C₁₆ alkyaryl;
b is 1, 2, 3, or 4; and d is 1, 2, 3, or 4;
Y is —C(O)—, —NRC(O)—, —NRC(S)—, —OC(O)—;
W is a bond, —(CH₂—O)ₜ—, —NRC(O)—, —NRC(O)NR—, —NRC(S)NR—, —NRC(O)O—, —OC(O)NR—, —OC(O)—, —C(O)NR—, or —C(O)O—, wherein t is an integer selected from the group consisting of 1, 2, 3, 4, 5, 6, 7, and 8;
G is

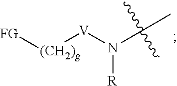

FG is a fluorescent dye moiety that emits in the near-infrared spectrum; V is —C(O)—, —NRC(O)—, —NRC(S)—, or—OC(O)—, and g is an integer selected from the group consisting of 1, 2, 3, 4, 5, and 6;
under the condition that when r is 0, then q and s are both 0 or both 1;
or a pharmaceutically acceptable salt thereof; under the proviso that if R′ is hydrogen.

In certain embodiments, the fluorescent dye moiety is:

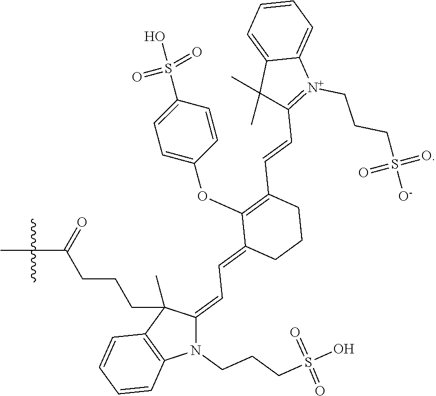 [SMILES: *C(=O)CCCC1(C)/C(=C\C=C2/CCCC(/C=C/C3=[N+](CCCS(=O)(=O)[O-])c4ccccc4C3(C)C)=C2Oc2ccc(S(=O)(=O)O)cc2)N(CCCS(=O)(=O)O)c2ccccc21]

In certain embodiments, the fluorescent dye moiety cannot be:

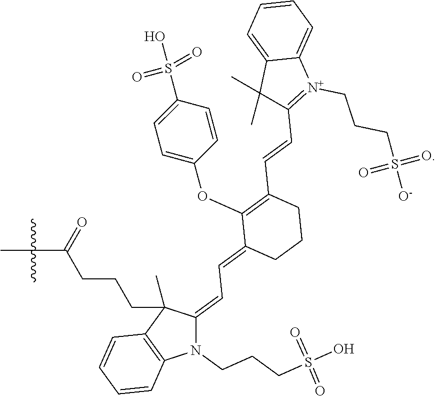 [SMILES: *C(=O)CCCC1(C)/C(=C\C=C2/CCCC(/C=C/C3=[N+](CCCS(=O)(=O)[O-])c4ccccc4C3(C)C)=C2Oc2ccc(S(=O)(=O)O)cc2)N(CCCS(=O)(=O)O)c2ccccc21]

In particular embodiments, the compound of formula (IV), the fluorescent dye moiety is:

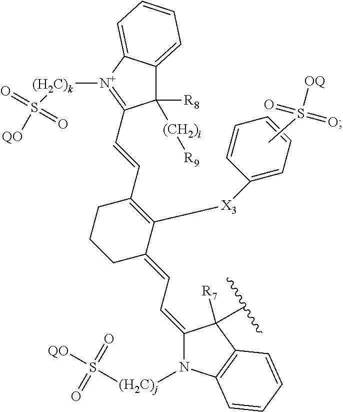

wherein:
i, j, and k are each an integer selected from the group consisting of 1, 2, 3, 4, 5, and 6;
X₃ is a single bond, —O—, or —S—;
R₇, R⁸, and R₉ are each independently hydrogen, C₁-C₄ unsubstituted or substituted alkyl, or -CO₂Q;
each Q is independently hydrogen, a metal ion, a negative charge, or a protecting group.

In certain embodiments, the compound of formula (IV) is

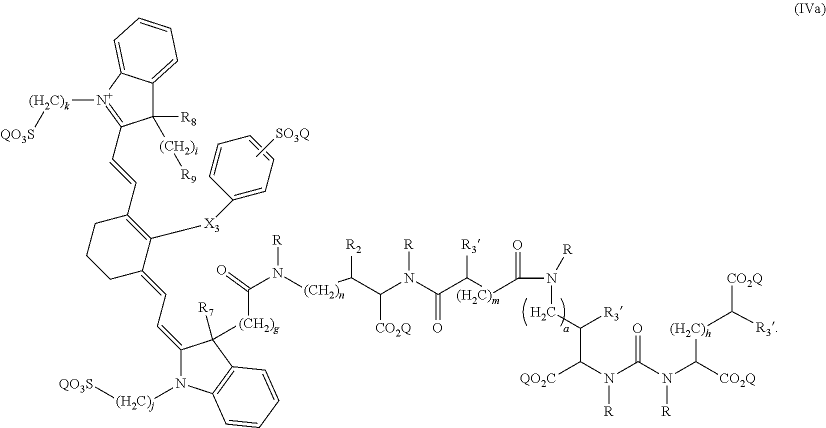

In certain embodiments, the compound of formula (IV) is:

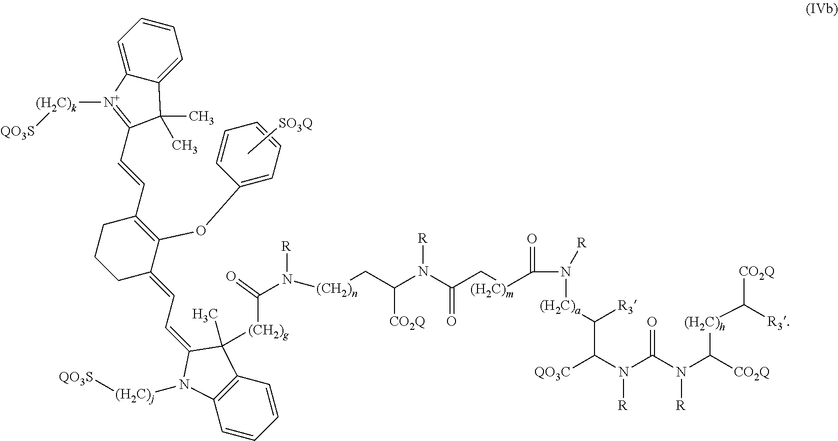

In particular embodiments, the compound of formula (IV) is selected from:

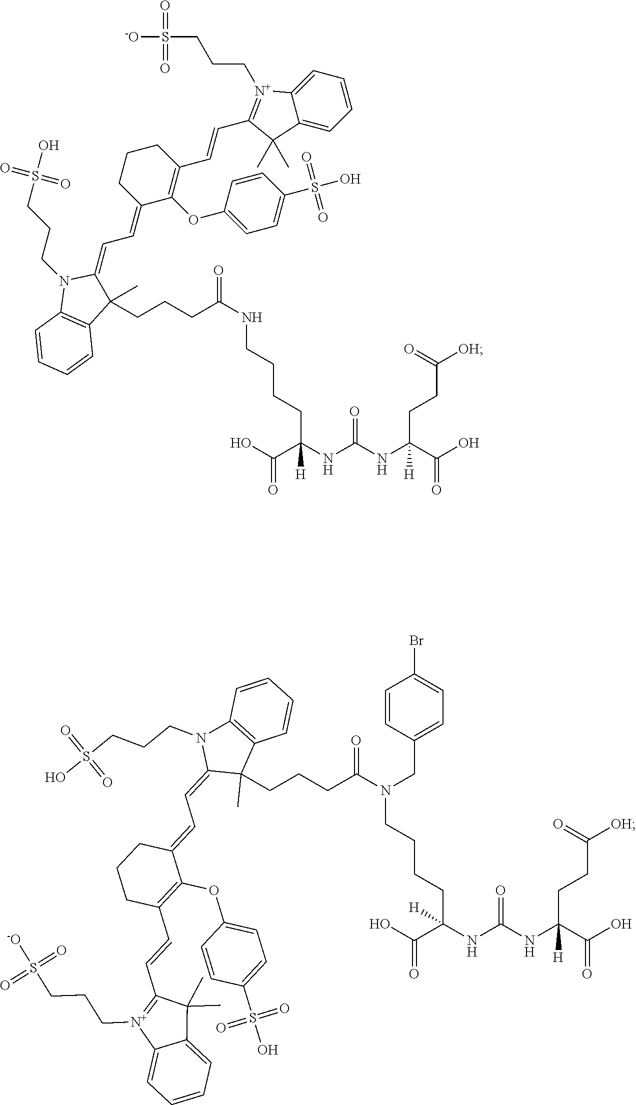

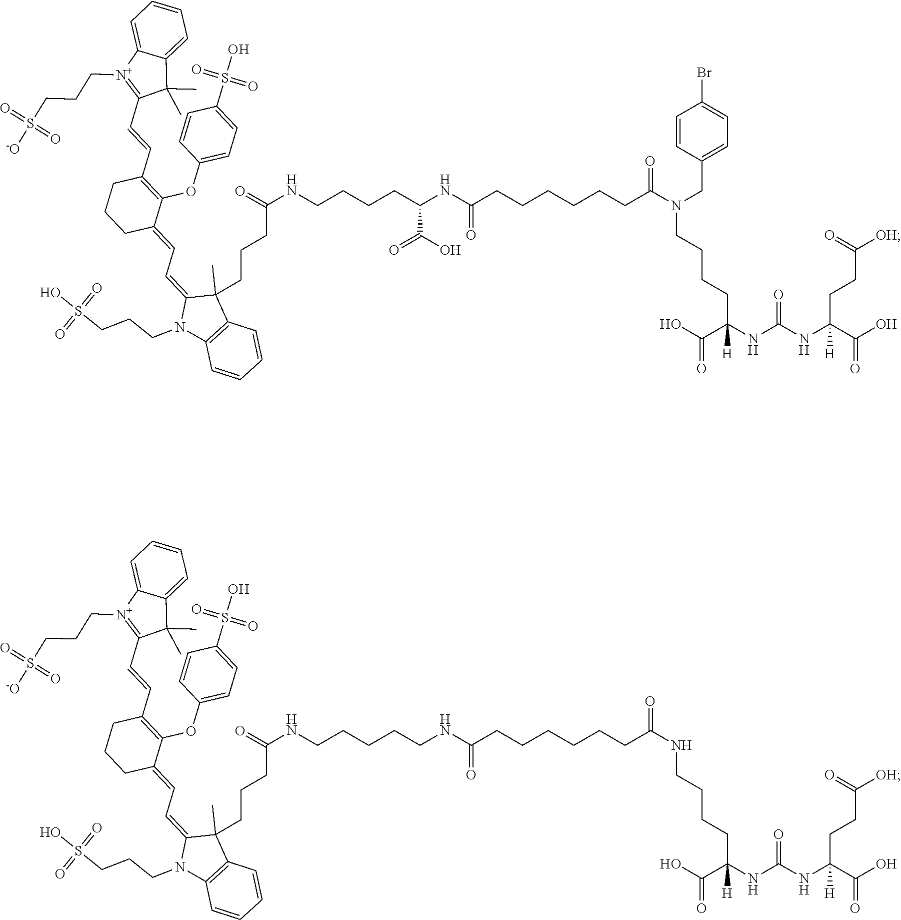

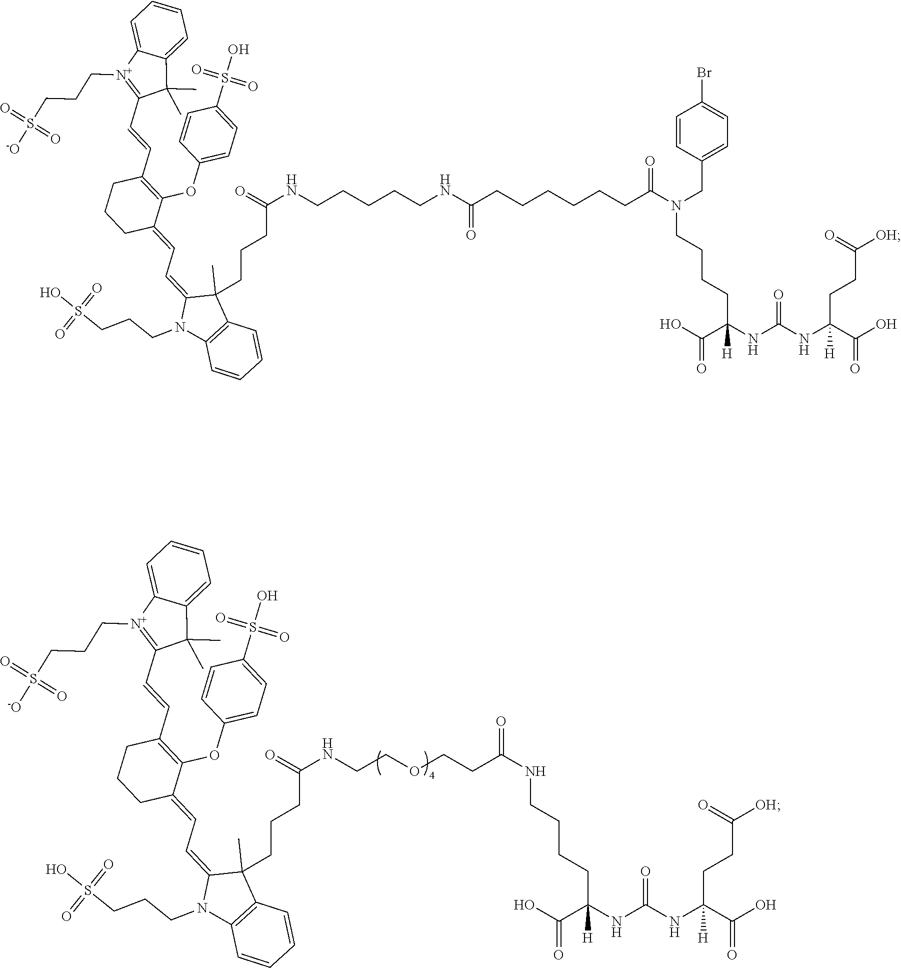

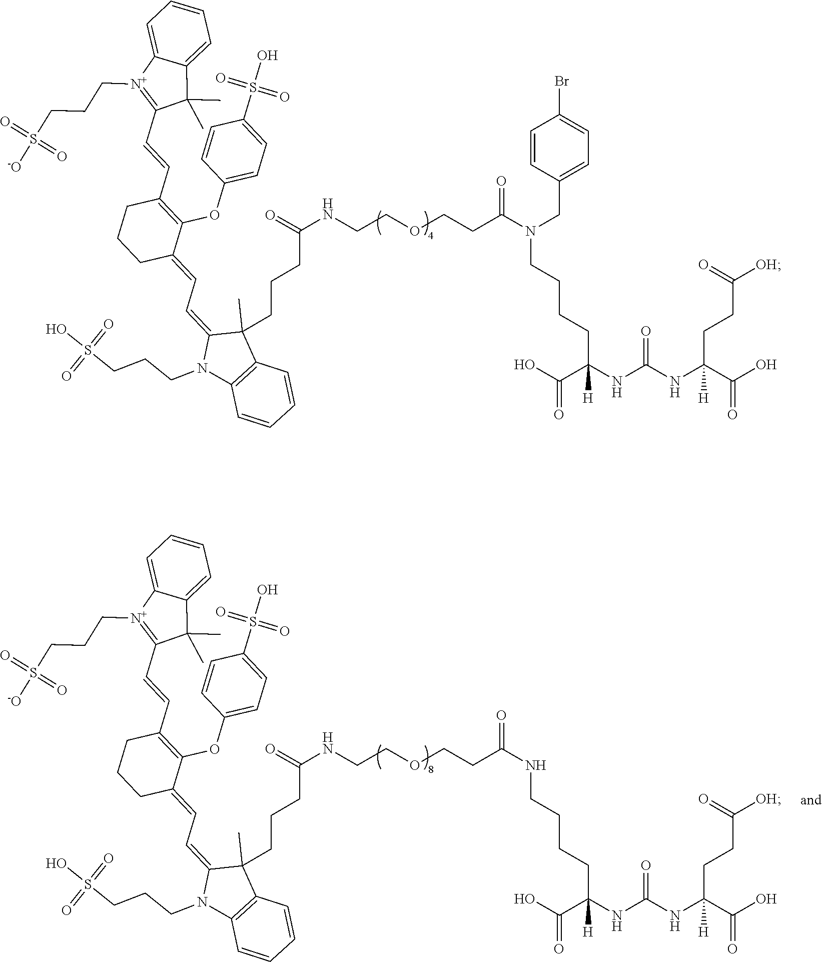

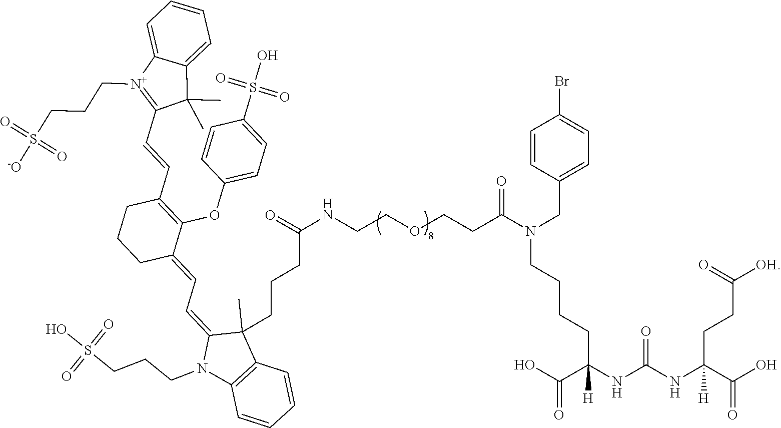

In other embodiments, the PSMA-targeting compound is

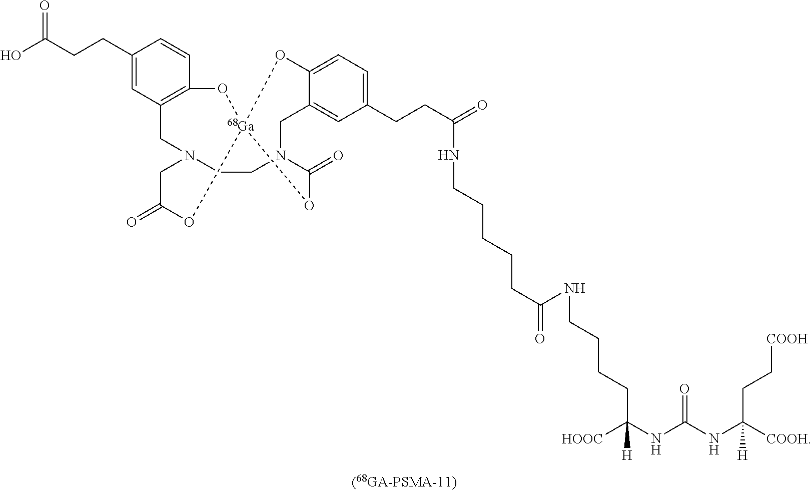

In general, the “effective amount” of an active agent refers to the amount necessary to elicit the desired biological response. As will be appreciated by those of ordinary skill in this art, the effective amount of an agent may vary depending on such factors as the desired biological endpoint, the agent to be delivered, the makeup of the pharmaceutical composition, the target tissue, and the like.

In certain embodiments, the presently disclosed method can be practiced in vitro or ex vivo by introducing, and preferably mixing, the compound and cell(s) or tumor(s) in a controlled environment, such as a culture dish or tube. The method can be practiced in vivo, in which case contacting means exposing the target in a subject to at least one compound of the presently disclosed subject matter, such as administering the compound to a subject via any suitable route. According to the presently disclosed subject matter, contacting may comprise introducing, exposing, and the like, the compound at a site distant to the cells to be contacted, and allowing the bodily functions of the subject, or natural (e.g., diffusion) or man-induced (e.g., swirling) movements of fluids to result in contact of the compound and the target.

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal (non-human) subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. In some embodiments, the subject is human. In other embodiments, the subject is non-human.

The present disclosure provides a pharmaceutical composition including one compound of formula (I-IV) alone or in combination with one or more additional therapeutic agents in admixture with a pharmaceutically acceptable excipient. One of skill in the art will recognize that the pharmaceutical compositions include the pharmaceutically acceptable salts of the compounds described above. Pharmaceutically acceptable salts are generally well known to those of ordinary skill in the art, and include salts of active compounds which are prepared with relatively nontoxic acids or bases, depending on the particular substituent moieties found on the compounds described herein. When compounds of the present disclosure contain relatively acidic functionalities, base addition salts can be obtained by contacting the neutral form of such compounds with a sufficient amount of the desired base, either neat or in a suitable inert solvent or by ion exchange, whereby one basic counterion (base) in an ionic complex is substituted for another. Examples of pharmaceutically acceptable base addition salts include sodium, potassium, calcium, ammonium, organic amino, or magnesium salt, or a similar salt.

When compounds of the present disclosure contain relatively basic functionalities, acid addition salts can be obtained by contacting the neutral form of such compounds with a sufficient amount of the desired acid, either neat or in a suitable inert solvent or by ion exchange, whereby one acidic counterion (acid) in an ionic complex is substituted for another. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-toluenesulfonic, citric, tartaric, methanesulfonic, and the like. Also included are salts of amino acids such as arginate and the like, and salts of organic acids like glucuronic or galactunoric acids and the like (see, for example, Berge et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Accordingly, pharmaceutically acceptable salts suitable for use with the presently disclosed subject matter include, by way of example but not limitation, acetate, benzenesulfonate, benzoate, bicarbonate, bitartrate, bromide, calcium edetate, camsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20^{th} ed.) Lippincott, Williams & Wilkins (2000). In therapeutic and/or diagnostic applications, the compounds of the disclosure can be formulated for a variety of modes of administration, including systemic and topical or localized administration. Techniques and formulations generally may be found in Remington: The Science and Practice of Pharmacy (20^{th} ed.) Lippincott, Williams & Wilkins (2000).

Depending on the specific conditions being treated, such agents may be formulated into liquid or solid dosage forms and administered systemically or locally. The agents may be delivered, for example, in a timed- or sustained-slow release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions, and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing, diluting, or dispersing substances, such as saline; preservatives, such as benzyl alcohol; absorption promoters; and fluorocarbons.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein. Generally, the compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, the bioavailability of the compound(s), the adsorption, distribution, metabolism, and excretion (ADME) toxicity of the compound(s), and the preference and experience of the attending physician.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

The term “combination” is used in its broadest sense and means that a subject is administered at least two agents, more particularly a compound described herein and at least one other therapeutic agent. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the compounds described herein can be administered alone or in combination with adjuvants that enhance stability of the compounds, alone or in combination with one or more therapeutic agents, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of a compound described herein and at least one additional therapeutic agent can be varied so long as the beneficial effects of the combination of these agents are achieved. Accordingly, the phrase “in combination with” refers to the administration of a compound described herein and at least one additional therapeutic agent either simultaneously, sequentially, or a combination thereof. Therefore, a subject administered a combination of a compound described herein and at least one additional therapeutic agent can receive a compound and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the compound described herein and at least one additional therapeutic agent are administered simultaneously, they can be administered to the subject as separate pharmaceutical compositions, each comprising either a compound or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a compound described herein and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

Qₐ/Q_{A}+Q_{b}/Q_{B}=Synergy Index(SI)
wherein:
Q_{A} is the concentration of a component A, acting alone, which produced an end point in relation to component A;
Qₐ is the concentration of component A, in a mixture, which produced an end point;
Q_{B} is the concentration of a component B, acting alone, which produced an end point in relation to component B; and
Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

While the following terms in relation to compounds of formula (I-IV) are believed to be well understood by one of ordinary skill in the art, the following definitions are set forth to facilitate explanation of the presently disclosed subject matter. These definitions are intended to supplement and illustrate, not preclude, the definitions that would be apparent to one of ordinary skill in the art upon review of the present disclosure.

The terms substituted, whether preceded by the term “optionally” or not, and substituent, as used herein, refer to the ability, as appreciated by one skilled in this art, to change one functional group for another functional group on a molecule, provided that the valency of all atoms is maintained. When more than one position in any given structure may be substituted with more than one substituent selected from a specified group, the substituent may be either the same or different at every position. The substituents also may be further substituted (e.g., an aryl group substituent may have another substituent off it, such as another aryl group, which is further substituted at one or more positions).

Where substituent groups or linking groups are specified by their conventional chemical formulae, written from left to right, they equally encompass the chemically identical substituents that would result from writing the structure from right to left, e.g., —CH₂O— is equivalent to —OCH₂—; —C(═O)O— is equivalent to —OC(═O)—; —OC(═O)NR— is equivalent to —NRC(═O)O—, and the like.

When the term “independently selected” is used, the substituents being referred to (e.g., R groups, such as groups R₁, R₂, and the like, or variables, such as “m” and “n”), can be identical or different. For example, both R₁ and R₂ can be substituted alkyls, or R₁ can be hydrogen and R₂ can be a substituted alkyl, and the like.

The terms “a,” “an,” or “a(n),” when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

A named “R” or group will generally have the structure that is recognized in the art as corresponding to a group having that name, unless specified otherwise herein. For the purposes of illustration, certain representative “R” groups as set forth above are defined below.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Unless otherwise explicitly defined, a “substituent group,” as used herein, includes a functional group selected from one or more of the following moieties, which are defined herein:

The term hydrocarbon, as used herein, refers to any chemical group comprising hydrogen and carbon. The hydrocarbon may be substituted or unsubstituted. As would be known to one skilled in this art, all valencies must be satisfied in making any substitutions. The hydrocarbon may be unsaturated, saturated, branched, unbranched, cyclic, polycyclic, or heterocyclic. Illustrative hydrocarbons are further defined herein below and include, for example, methyl, ethyl, n-propyl, isopropyl, cyclopropyl, allyl, vinyl, n-butyl, tert-butyl, ethynyl, cyclohexyl, and the like.

The term “alkyl,” by itself or as part of another substituent, means, unless otherwise stated, a straight (i.e., unbranched) or branched chain, acyclic or cyclic hydrocarbon group, or combination thereof, which may be fully saturated, mono- or polyunsaturated and can include di- and multivalent groups, having the number of carbon atoms designated (i.e., C₁₋₁₀ means one to ten carbons, including 1, 2, 3, 4, 5, 6, 7, 8, 9, and 10 carbons). In particular embodiments, the term “alkyl” refers to C₁₋₂₀ inclusive, including 1, 2, 3, 4, 5, 6, 7, 8, 9, 10, 11, 12, 13, 14, 15, 16, 17, 18, 19, and 20 carbons, linear (i.e., “straight-chain”), branched, or cyclic, saturated or at least partially and in some cases fully unsaturated (i.e., alkenyl and alkynyl) hydrocarbon radicals derived from a hydrocarbon moiety containing between one and twenty carbon atoms by removal of a single hydrogen atom.

Representative saturated hydrocarbon groups include, but are not limited to, methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, n-octyl, n-decyl, n-undecyl, dodecyl, cyclohexyl, (cyclohexyl)methyl, cyclopropylmethyl, and homologs and isomers thereof.

“Branched” refers to an alkyl group in which a lower alkyl group, such as methyl, ethyl, or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁-8 branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, cyano, and mercapto.

The term “heteroalkyl,” by itself or in combination with another term, means, unless otherwise stated, a stable straight or branched chain having from 1 to 20 carbon atoms or heteroatoms or a cyclic hydrocarbon group having from 3 to 10 carbon atoms or heteroatoms, or combinations thereof, consisting of at least one carbon atom and at least one heteroatom selected from O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CHO—CH₃, —Si(CH₃)₃, —CH₂—CH═N—OCH₃, —CH═CH—N(CH₃)— CH₃, O—CH₃, —O—CH₂—CH₃, and —CN. Up to two or three heteroatoms may be consecutive, such as, for example, —CH₂—NH—OCH₃ and —CH₂—O—Si(CH₃)₃.

As described above, heteroalkyl groups, as used herein, include those groups that are attached to the remainder of the molecule through a heteroatom, such as —C(O)NR′, —NR′R″, —OR′, —SR, —S(O)R, and/or —S(O₂)R′. Where “heteroalkyl” is recited, followed by recitations of specific heteroalkyl groups, such as —NR′R or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, unsubstituted alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl, and fused ring systems, such as dihydro- and tetrahydronaphthalene, and the like.

The term “cycloalkylalkyl,” as used herein, refers to a cycloalkyl group as defined hereinabove, which is attached to the parent molecular moiety through an alkylene moiety, also as defined above, e.g., a C₁20 alkylene moiety. Examples of cycloalkylalkyl groups include cyclopropylmethyl and cyclopentylethyl.

The terms “cycloheteroalkyl” or “heterocycloalkyl” refer to a non-aromatic ring system, unsaturated or partially unsaturated ring system, such as a 3- to 10-member substituted or unsubstituted cycloalkyl ring system, including one or more heteroatoms, which can be the same or different, and are selected from nitrogen (N), oxygen (O), sulfur (S), phosphorus (P), and silicon (Si), and optionally can include one or more double bonds.

The cycloheteroalkyl ring can be optionally fused to or otherwise attached to other cycloheteroalkyl rings and/or non-aromatic hydrocarbon rings. Heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocylic refers to a non-aromatic 5-, 6-, or 7-membered ring or a polycyclic group wherein at least one ring atom is a heteroatom selected from O, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Representative cycloheteroalkyl ring systems include, but are not limited to pyrrolidinyl, pyrrolinyl, imidazolidinyl, imidazolinyl, pyrazolidinyl, pyrazolinyl, piperidinyl, piperazinyl, indolinyl, quinuclidinyl, morpholinyl, thiomorpholinyl, thiadiazinanyl, tetrahydrofuranyl, and the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively.

An unsaturated hydrocarbon has one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl.”

More particularly, the term “alkenyl” as used herein refers to a monovalent group derived from a C₂₋₂₀ inclusive straight or branched hydrocarbon moiety having at least one carbon-carbon double bond by the removal of a single hydrogen molecule. Alkenyl groups include, for example, ethenyl (i.e., vinyl), propenyl, butenyl, 1-methyl-2-buten-1-yl, pentenyl, hexenyl, octenyl, allenyl, and butadienyl.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “alkynyl” as used herein refers to a monovalent group derived from a straight or branched C₂₋₂₀ hydrocarbon of a designed number of carbon atoms containing at least one carbon-carbon triple bond. Examples of “alkynyl” include ethynyl, 2-propynyl (propargyl), 1-propynyl, pentynyl, hexynyl, and heptynyl groups, and the like.

The term “alkylene” by itself or a part of another substituent refers to a straight or branched bivalent aliphatic hydrocarbon group derived from an alkyl group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched, or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —CH₂CH₂CH₂CH₂—, —CH₂CH═CHCH₂—, —CH₂CsCCH₂—, —CH₂CH₂CH(CH₂CH₂CH₃)CH₂—, —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons. Typically, an alkyl (or alkylene) group will have from 1 to 24 carbon atoms, with those groups having 10 or fewer carbon atoms being some embodiments of the present disclosure. A “lower alkyl” or “lower alkylene” is a shorter chain alkyl or alkylene group, generally having eight or fewer carbon atoms.

The term “heteroalkylene” by itself or as part of another substituent means a divalent group derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms also can occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—.

The term “aryl” means, unless otherwise stated, an aromatic hydrocarbon substituent that can be a single ring or multiple rings (such as from 1 to 3 rings), which are fused together or linked covalently. The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent forms of aryl and heteroaryl, respectively.

For brevity, the term “aryl” when used in combination with other terms (e.g., aryloxy, arylthioxy, arylalkyl) includes both aryl and heteroaryl rings as defined above. Thus, the terms “arylalkyl” and “heteroarylalkyl” are meant to include those groups in which an aryl or heteroaryl group is attached to an alkyl group (e.g., benzyl, phenethyl, pyridylmethyl, furylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). The term “haloaryl,” however, as used herein is meant to cover only aryls substituted with one or more halogens.

Where a heteroalkyl, heterocycloalkyl, or heteroaryl includes a specific number of members (e.g., “3 to 7 membered”), the term “member” refers to a carbon or heteroatom.

Further, a structure represented generally by the formula:

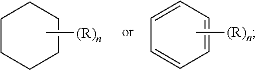

as used herein refers to a ring structure, for example, but not limited to a 3-carbon, a 4-carbon, a 5-carbon, a 6-carbon, a 7-carbon, and the like, aliphatic and/or aromatic cyclic compound, including a saturated ring structure, a partially saturated ring structure, and an unsaturated ring structure, comprising a substituent R group, wherein the R group can be present or absent, and when present, one or more R groups can each be substituted on one or more available carbon atoms of the ring structure. The presence or absence of the R group and number of R groups is determined by the value of the variable “n,” which is an integer generally having a value ranging from 0 to the number of carbon atoms on the ring available for substitution. Each R group, if more than one, is substituted on an available carbon of the ring structure rather than on another R group. For example, the structure above where n is 0 to 2 would comprise compound groups including, but not limited to:

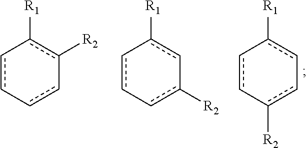

and the like.

A dashed line representing a bond in a cyclic ring structure indicates that the bond can be either present or absent in the ring. That is, a dashed line representing a bond in a cyclic ring structure indicates that the ring structure is selected from a saturated ring structure, a partially saturated ring structure, and an unsaturated ring structure.

The symbol ( [symbol] ) denotes the point of attachment of a moiety to the remainder of the molecule.

When a named atom of an aromatic ring or a heterocyclic aromatic ring is defined as being “absent,” the named atom is replaced by a direct bond.

Each of above terms (e.g., “alkyl,” “heteroalkyl,” “cycloalkyl, and “heterocycloalkyl”, “aryl,” “heteroaryl,” “phosphonate,” and “sulfonate” as well as their divalent derivatives) are meant to include both substituted and unsubstituted forms of the indicated group. Optional substituents for each type of group are provided below.

Substituents for alkyl, heteroalkyl, cycloalkyl, heterocycloalkyl monovalent and divalent derivative groups (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, =N—OR′, —NR′R″, —SR′, -halogen, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″)=NR′″, —S(O)R′, —S(O)₂R′, —S(O)₂NR′R″, —NRSO₂R′, —CN, CF₃, fluorinated C₁₋₄ alkyl, and —NO₂ in a number ranging from zero to (2m′+1), where m′ is the total number of carbon atoms in such groups. R′, R″, R′″ and R″″ each may independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. As used herein, an “alkoxy” group is an alkyl attached to the remainder of the molecule through a divalent oxygen. When a compound of the disclosure includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for alkyl groups above, exemplary substituents for aryl and heteroaryl groups (as well as their divalent derivatives) are varied and are selected from, for example: halogen, —OR′, —NR′R″, —SR′, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″R′″)=NR″ ″, —NR—C(NR′R″)=NR′″—S(O)R′, —S(O)₂R′, —S(O)₂NR′R″, —NRSO₂R′, —CN and —NO₂, —R′, —N₃, —CH(Ph)₂, fluoro(C₁₋₄)alkoxo, and fluoro(C₁₋₄)alkyl, in a number ranging from zero to the total number of open valences on aromatic ring system; and where R′, R″, R′″ and R″″ may be independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the disclosure includes more than one R group, for example, each of the R groups is independently selected as are each R′, R″, R′″ and R″″ groups when more than one of these groups is present.

Two of the substituents on adjacent atoms of aryl or heteroaryl ring may optionally form a ring of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4.

One of the single bonds of the new ring so formed may optionally be replaced with a double bond. Alternatively, two of the substituents on adjacent atoms of aryl or heteroaryl ring may optionally be replaced with a substituent of the formula —(CRR′)ₛ—X′— (C″R′″)_{d}—, where s and d are independently integers of from 0 to 3, and X′ is —O—, —NR′—, —S—, —S(O)—, —S(O)₂—, or —S(O)₂NR′—. The substituents R, R′, R″ and R′″ may be independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “acyl” refers to an organic acid group wherein the —OH of the carboxyl group has been replaced with another substituent and has the general formula RC(═O)—, wherein R is an alkyl, alkenyl, alkynyl, aryl, carbocylic, heterocyclic, or aromatic heterocyclic group as defined herein). As such, the term “acyl” specifically includes arylacyl groups, such as a 2-(furan-2-yl)acetyl)- and a 2-phenylacetyl group. Specific examples of acyl groups include acetyl and benzoyl. Acyl groups also are intended to include amides, —RC(═O)NR′, esters, —RC(═O)OR′, ketones, —RC(═O)R′, and aldehydes, —RC(═O)H.

The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include C₁₋₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, tert-butoxyl, and n-pentoxyl, neopentoxyl, n-hexoxyl, and the like.

The term “alkoxyalkyl” as used herein refers to an alkyl-O-alkyl ether, for example, a methoxyethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl, i.e., C₆H₅—CH₂—O—. An aralkyloxyl group can optionally be substituted.

“Alkoxycarbonyl” refers to an alkyl-O—C(═O)— group. Exemplary alkoxycarbonyl groups include methoxycarbonyl, ethoxycarbonyl, butyloxycarbonyl, and tert-butyloxycarbonyl.

“Aryloxycarbonyl” refers to an aryl-O—C(═O)— group. Exemplary aryloxycarbonyl groups include phenoxy- and naphthoxy-carbonyl.

“Aralkoxycarbonyl” refers to an aralkyl-O—C(═O)— group. An exemplary aralkoxycarbonyl group is benzyloxycarbonyl.

“Carbamoyl” refers to an amide group of the formula —C(═O)NH₂. “Alkylcarbamoyl” refers to a R′RN—C(═O)— group wherein one of R and R′ is hydrogen and the other of R and R′ is alkyl and/or substituted alkyl as previously described. “Dialkylcarbamoyl” refers to a R′RN—C(═O)— group wherein each of R and R′ is independently alkyl and/or substituted alkyl as previously described.

The term carbonyldioxyl, as used herein, refers to a carbonate group of the formula —O—C(═O)—OR.

“Acyloxyl” refers to an acyl-O— group wherein acyl is as previously described.

The term “amino” refers to the —NH₂ group and also refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups, respectively.

An “aminoalkyl” as used herein refers to an amino group covalently bound to an alkylene linker. More particularly, the terms alkylamino, dialkylamino, and trialkylamino as used herein refer to one, two, or three, respectively, alkyl groups, as previously defined, attached to the parent molecular moiety through a nitrogen atom. The term alkylamino refers to a group having the structure —NHR′ wherein R′ is an alkyl group, as previously defined; whereas the term dialkylamino refers to a group having the structure —NR′R″, wherein R′ and R″ are each independently selected from alkyl groups. The term trialkylamino refers to a group having the structure —NR′R″R′″, wherein R′, R″, and R′″ are each independently selected from alkyl groups. Additionally, R′, R″, and/or R′″taken together may optionally be —(CH₂)ₖ— where k is an integer from 2 to 6. Examples include, but are not limited to, methylamino, dimethylamino, ethylamino, diethylamino, diethylaminocarbonyl, methylethylamino, isopropylamino, piperidino, trimethylamino, and propylamino.

The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms alkylthioether and thioalkoxyl refer to a saturated (i.e., alkyl-S—) or unsaturated (i.e., alkenyl-S— and alkynyl-S—) group attached to the parent molecular moiety through a sulfur atom. Examples of thioalkoxyl moieties include, but are not limited to, methylthio, ethylthio, propylthio, isopropylthio, n-butylthio, and the like.

“Acylamino” refers to an acyl-NH— group wherein acyl is as previously described. “Aroylamino” refers to an aroyl-NH— group wherein aroyl is as previously described.

The term “carbonyl” refers to the —C(═O)— group, and can include an aldehyde group represented by the general formula R—C(═O)H.

The term “carboxyl” refers to the —COOH group. Such groups also are referred to herein as a “carboxylic acid” moiety.

The term “cyano” refers to the —C≡N group.

The terms “halo,” “halide,” or “halogen” as used herein refer to fluoro, chloro, bromo, and iodo groups. Additionally, terms such as “haloalkyl,” are meant to include monohaloalkyl and polyhaloalkyl. For example, the term “halo(C₁₋₄)alkyl” is mean to include, but not be limited to, trifluoromethyl, 2,2,2-trifluoroethyl, 4-chlorobutyl, 3-bromopropyl, and the like.

The term “hydroxyl” refers to the —OH group.

The term “hydroxyalkyl” refers to an alkyl group substituted with an —OH group.

The term “mercapto” refers to the —SH group.

The term “oxo” as used herein means an oxygen atom that is double bonded to a carbon atom or to another element.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term thiohydroxyl or thiol, as used herein, refers to a group of the formula —SH.

More particularly, the term “sulfide” refers to compound having a group of the formula —SR.

The term “sulfone” refers to compound having a sulfonyl group —S(O₂)R.

The term “sulfoxide” refers to a compound having a sulfinyl group —S(O)R.

The term ureido refers to a urea group of the formula —NH—CO—NH₂.

Throughout the specification and claims, a given chemical formula or name shall encompass all tautomers, congeners, and optical- and stereoisomers, as well as racemic mixtures where such isomers and mixtures exist.

Certain compounds of the present disclosure may possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as D- or L- for amino acids, and individual isomers are encompassed within the scope of the present disclosure. The compounds of the present disclosure do not include those which are known in art to be too unstable to synthesize and/or isolate. The present disclosure is meant to include compounds in racemic, scalemic, and optically pure forms. Optically active (R)- and (S)-, or D- and L-isomers may be prepared using chiral synthons or chiral reagents, or resolved using conventional techniques. When the compounds described herein contain olefenic bonds or other centers of geometric asymmetry, and unless specified otherwise, it is intended that the compounds include both E and Z geometric isomers.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium and which are readily converted from one isomeric form to another.

Unless otherwise stated, structures depicted herein are also meant to include compounds which differ only in the presence of one or more isotopically enriched atoms. For example, compounds having the present structures with the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present disclosure, whether radioactive or not, are encompassed within the scope of the present disclosure.

The compounds of the present disclosure may exist as salts. The present disclosure includes such salts. Examples of applicable salt forms include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures, succinates, benzoates, and salts with amino acids such as glutamic acid. These salts may be prepared by methods known to those skilled in art. Also included are base addition salts such as sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent or by ion exchange. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

In addition to salt forms, the present disclosure provides compounds, which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

The term “protecting group” refers to chemical moieties that block some or all reactive moieties of a compound and prevent such moieties from participating in chemical reactions until the protective group is removed, for example, those moieties listed and described in T. W. Greene, P.G.M. Wuts, Protective Groups in Organic Synthesis, 3rd ed. John Wiley & Sons (1999). It may be advantageous, where different protecting groups are employed, that each (different) protective group be removable by a different means. Protective groups that are cleaved under totally disparate reaction conditions allow differential removal of such protecting groups. For example, protective groups can be removed by acid, base, and hydrogenolysis. Groups such as trityl, dimethoxytrityl, acetal and tert-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, without limitation, methyl, ethyl, and acetyl in the presence of amines blocked with acid labile groups such as tert-butyl carbamate or with carbamates that are both acid and base stable but hydrolytically removable.

Carboxylic acid and hydroxy reactive moieties may also be blocked with hydrolytically removable protective groups, such as the benzyl group, while amine groups capable of hydrogen bonding with acids may be blocked with base labile groups such as Fmoc. Carboxylic acid reactive moieties may be blocked with oxidatively-removable protective groups such as 2,4-dimethoxybenzyl, while co-existing amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a palladium(O)-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Representative protecting groups include, but are not limited to, benzyl, p-methoxybenzyl (PMB), tertiary butyl (t-Bu), methoxymethyl (MOM), methoxyethoxymethyl (MEM), methylthiomethyl (MTM), tetrahydropyranyl (THP), tetrahydrofuranyl (THF), benzyloxymethyl (BOM), trimethylsilyl (TMS), triethylsilyl (TES), t-butyldimethylsilyl (TBDMS), and triphenylmethyl (trityl, Tr), and include moieties having the following chemical structures:

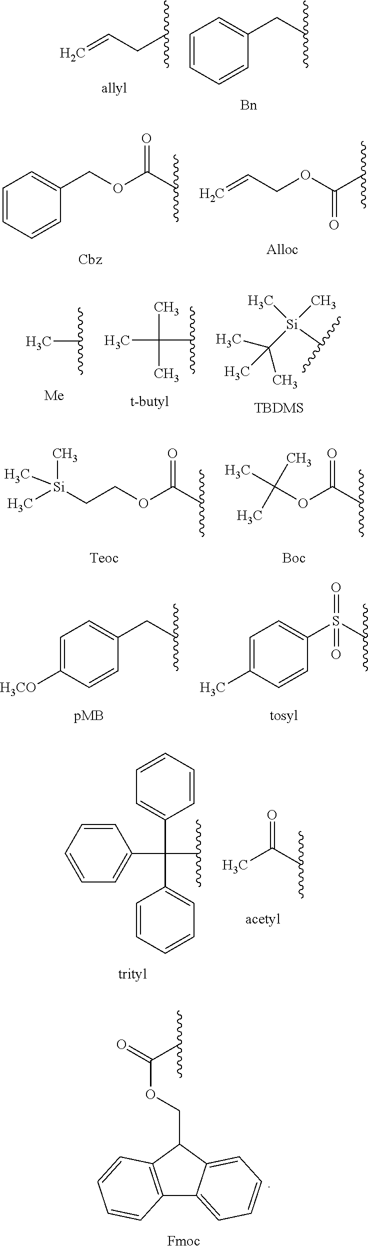

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Representative TSPO-Targeting Compounds for Evaluation of Injuries in the Peripheral Nervous System

### Near-Infrared Imaging

Ex-vivo near-infrared (NIR) imaging of different nerve injuries was performed using NIR-labeled DPA-713. Lewis rats received 10-nmol doses of DPA-713-dye via tail vein injection prior to sacrifice, hip disarticulation, and hindlimb imaging using a LI-COR Pearl Impulse Small Animal Imaging System (LI-COR Biosciences). Hindlimb groups included: 4-weeks after sciatic nerve transection; 4-weeks after sciatic nerve transection with epineurial suture repair; 2-weeks after sciatic nerve transection with epineurial suture repair; 2-weeks after common peroneal nerve crush; and uninjured control.

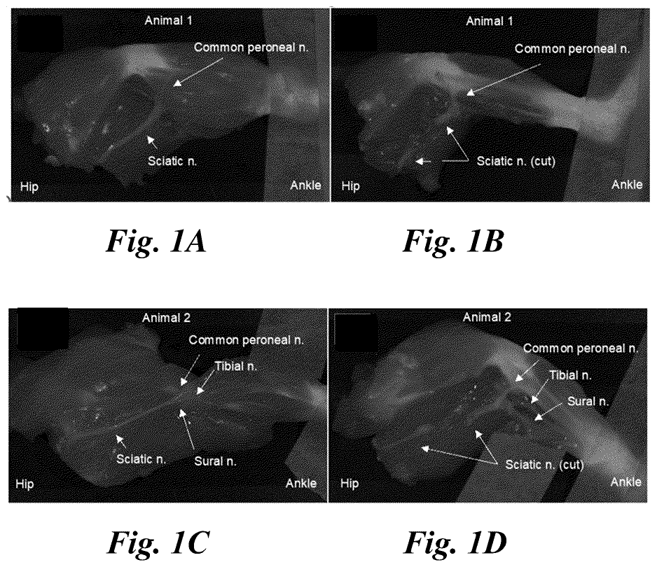

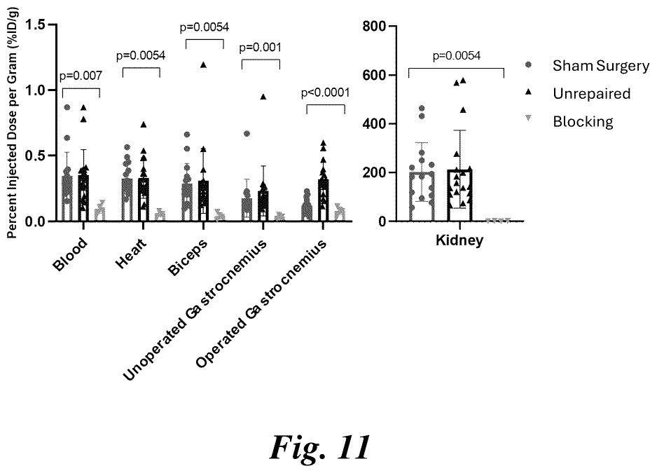

We demonstrated low baseline DPA-713-dye uptake in uninjured sciatic nerves (FIG. 1A, FIG. 11C), and a consistent increase in DPA-713-dye uptake within denervation portions of nerve after sciatic transection without repair (FIG. 1B, FIG. 1D).

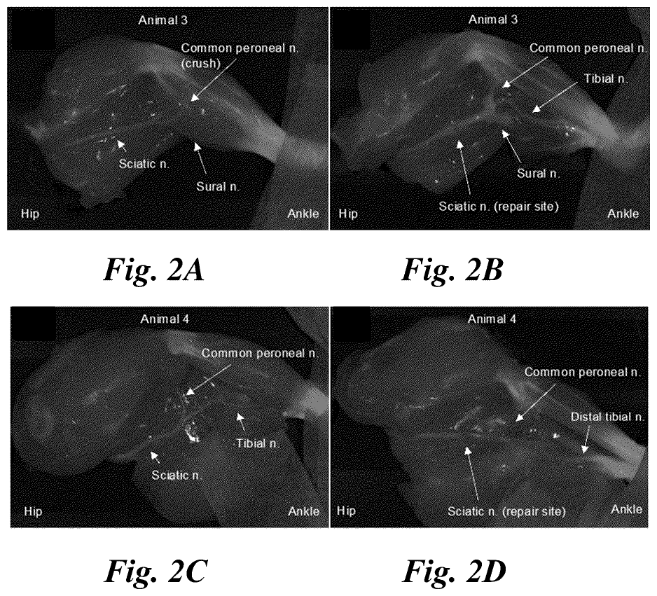

At 2 weeks post-injury, we also demonstrated increase DPAY-713-dye uptake distal to the site of common perineal nerve crush (FIG. 2A) and distal to the site of sciatic nerve transection with repair (FIG. 2B). At 4 weeks following nerve repair, we demonstrated resolution of DPA-713-dye uptake in regeneration portions of nerve (FIG. 2D) toward baseline of the uninjured limb (FIG. 2C).

### Biodistribution

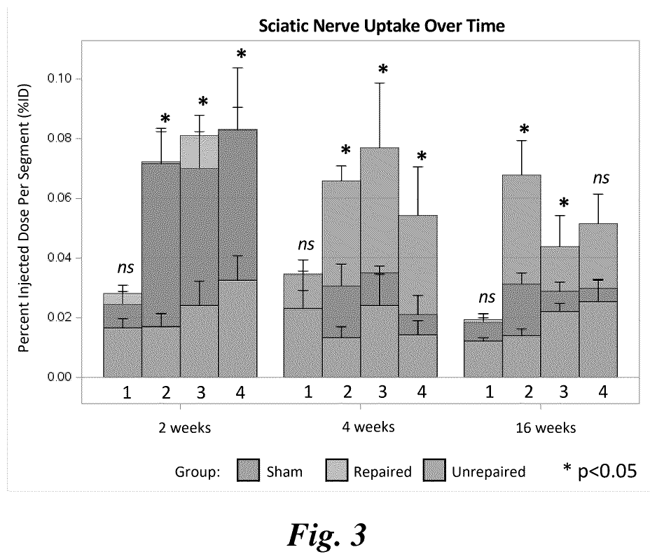

We next evaluated the biodistribution of ¹²⁴I-iodo-DPA713 after sciatic nerve injury. Forty-nine Lewis rats received unilateral sciatic nerve transection without repair, transection with repair, or sham surgery and underwent biodistribution evaluations at 2 weeks, 4 weeks, or 16 weeks post-injury (n=16 per timepoint). Animals received 109+/−50 μCi of ¹²⁴I-Iodo-DPA713 intravenously prior to euthanasia and harvest of the sciatic nerve. The nerve was harvested in 4 segments to measure uptake along the nerve: 2 segments proximal to the injury site and 2 segments distal to the injury site. Activity in each segment was calculated using an automated gamma counter (LKB Compugamma CS 1282) and expressed as percent injected dose per gram (% ID/segment). Similar to the NIR imaging finding, uptake was elevated just proximal to the injury site and distal to the injury site at 2 weeks post-injury in nerves that had undergone either transection without repair or transection with repair (FIG. 3). By 4 weeks post-injury, nerve uptake resolved toward sham surgery in repaired nerves but remained elevated in unrepaired nerves (FIG. 3). This trend persisted at 16 weeks post-injury (FIG. 3).

Large animal ¹²⁴I-iodo-DPA713 PET-CT

A female Yorkshire pig at age 5 months underwent proximal right median nerve transection with repair and left median nerve transection without repair at the proximal humerus, 12 cm from the olecranon. The median nerve size, regenerative capacity, and regenerative distance between injury and forearm flexor muscles are analogous to humans. Scholz et al., 2010; Smith et al., 2022.

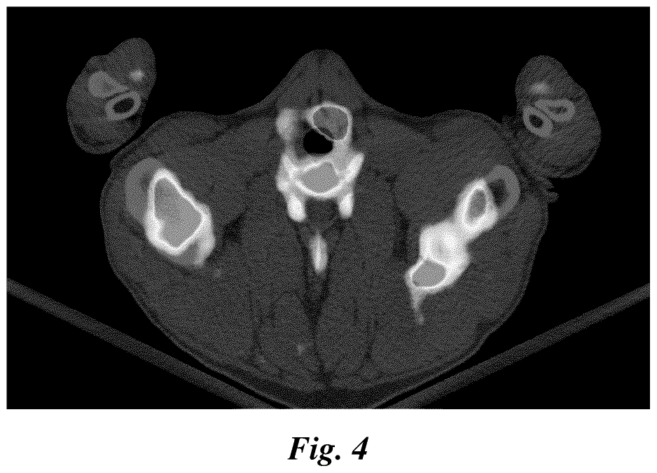

At 16-weeks post-injury and a weight of 68.8 kg, the animal received intravenous injection of 3 mCi of ¹²⁴I-iodo-DPA713 prior to whole-body imaging on a clinical PET-CT scanner (Siemens Biography mCT PET-CT). Increased uptake ¹²⁴I-iodo-DPA713 localized to the bilateral distal forearms in regions corresponding the expected location of the median nerves (FIG. 4).

### Large Animal Histology

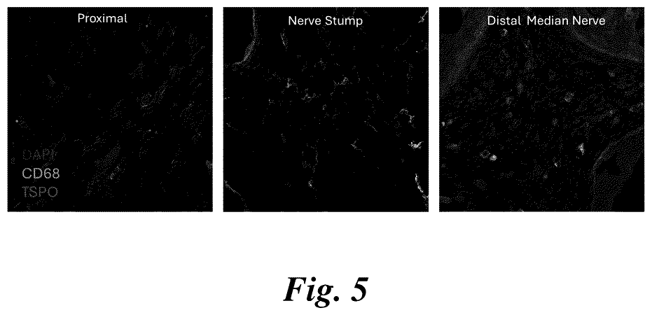

The Yorkshire pig was sacrificed following PET-CT and the median nerves were harvested for histology. Immunohistochemical analysis identified co-localization between the macrophage marker CD68 and TSPO at the neuroma stump, as well as in the distal denervated median nerve (FIG. 5).

### Human Histology

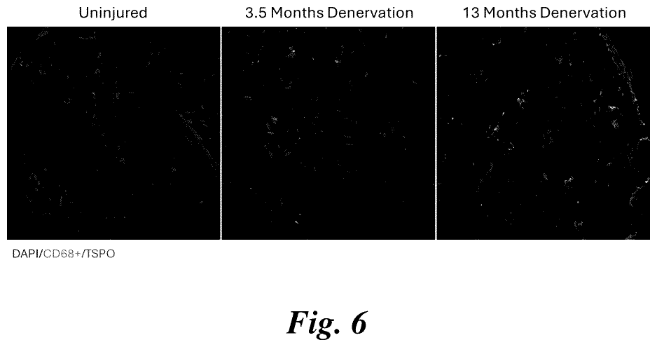

TSPO expression was also evaluated in human major peripheral nerve biopsies collected from patients with nerve injuries at the time of nerve reconstruction. As in the pig histology, TSPO+/CD68+ macrophages were identified in degenerated nerves several months after injury (FIG. 6).

### Example 2

### Representative PSMA-Targeting Compounds for Evaluation of Injuries in the Peripheral Nervous System

### BACKGROUND

In this Example, we sought to investigate the clinical GCPII-binding PET agents to evaluate muscle denervation. We demonstrate sustained uptake of ¹⁸F-DCFPyL and ⁶⁸Ga-PSMA-11 in both rodent and pig nerve injury models at least 16 weeks after muscle denervation. Given its established safety and clinical availability, GCPII-based PET has high translational potential in the care of patients with complex PNS injuries.

### Near-Infrared Imaging

Lewis rats received diverse injuries to the sciatic nerve and its branches. At various timepoints after injury, animals rats received 10 nanomole doses of the fluorescent PSMA-targeting ligand YC27 via tail vein injection prior to sacrifice and hindlimb harvest. The skin was removed and biceps femoris dissected away to expose the sciatic nerve for evaluation using a Pearl Impulse Near-Infrared (NIR) Imager.

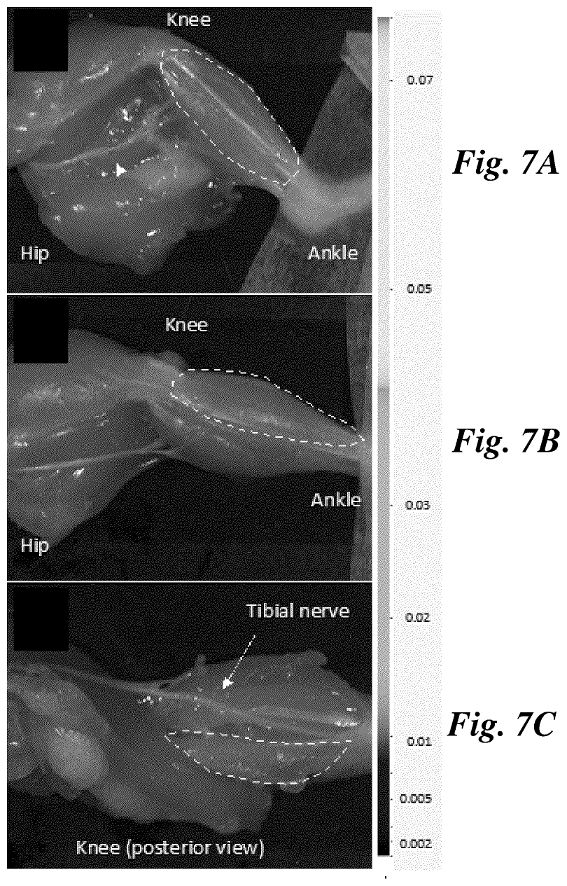

At 2 weeks post-injury, sciatic nerve transection with repair resulted in elevated YC27 uptake in anterior and posterior leg compartment musculature (FIG. 7A). A complete crush injury of the proximal common peroneal nerve resulted in YC27 uptake in the denervated anterior compartment muscles only (FIG. 7B). Selective transection of the medial gastrocnemius muscle branch resulted in specific YC27 uptake to the denervated medial head with sparing of the uninjured lateral head (FIG. 7C).

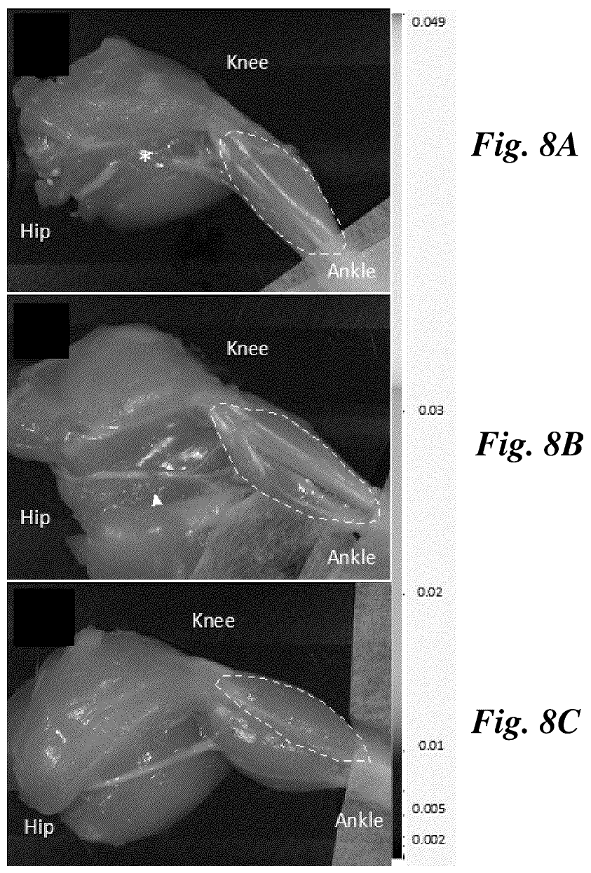

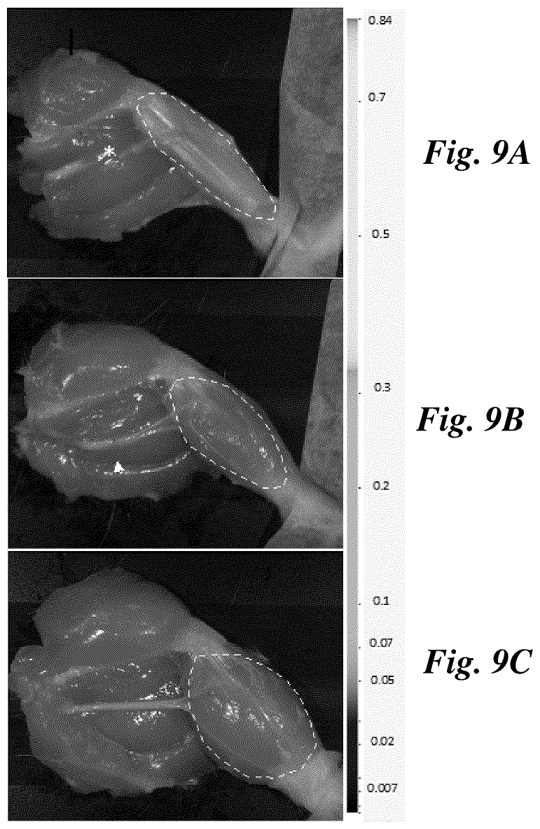

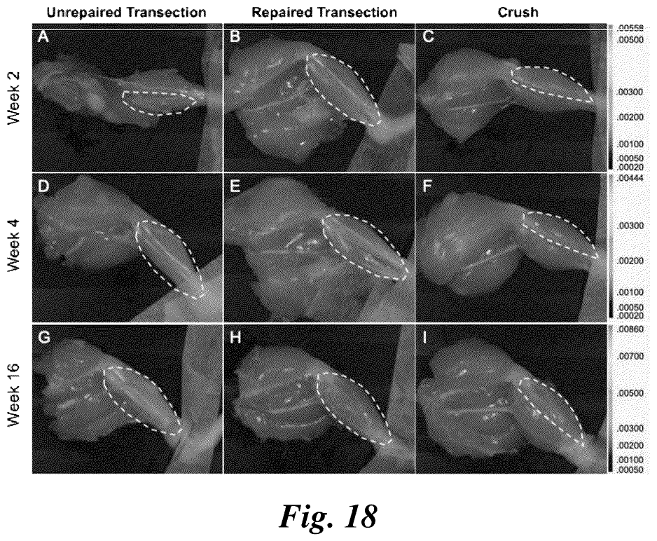

At 4 weeks post-injury, YC27 uptake remained elevated in affected muscles after transection without repair (FIG. 8A) and transection with repair (FIG. 8B). Uptake resolved 4 weeks after common peroneal crush, consistent with the expected course of re-innervation (FIG. 8C). Similarly, YC27 uptake remained high 16 weeks after sciatic nerve transection without repair (FIG. 9A but resolved in the limb after sciatic transection with repair (FIG. 9B). Muscle uptake also resolves towards baseline 16 weeks after common perineal nerve crush (FIG. 9C). See also FIG. 18.

### Biodistribution

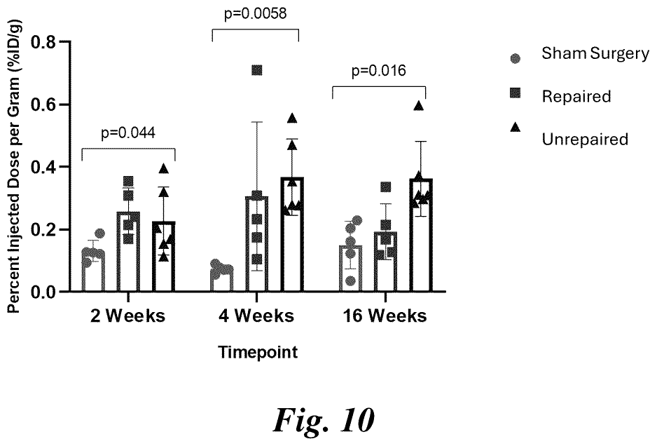

Forty-eight rats (24 Male, 24 Female) received unilateral sciatic nerve transection without repair, transection with repair, or sham surgery and underwent biodistribution evaluations at 2 weeks, 4 weeks, or 16 weeks post-injury (n=16 per timepoint). Animals received 140+/−45 μCi of ¹⁸F-DCFPyL intravenously prior to euthanasia and harvest of gastrocnemius muscles (FIG. 10). Activity in each muscle was calculated using an automated gamma counter (LKB Compugamma CS 1282) and expressed as percent injected dose per gram (% ID/g).

### Blocking Studies

Four additional animals received co-injection of 100±2 μCi of ¹⁸F-DCFPyL and 100 mg/kg of ZJ-43, a potent competitive inhibitor of GCPII binding, 4 weeks after sciatic nerve transection without repair. ZJ-43 eliminated differences in ¹⁸F-DCFPyL activity between denervated and non-denervated muscles, consistent with ¹⁸F-DCFPyL specificity for muscle GCPII (FIG. 11).

### Serial Small-Animal PET-MRI

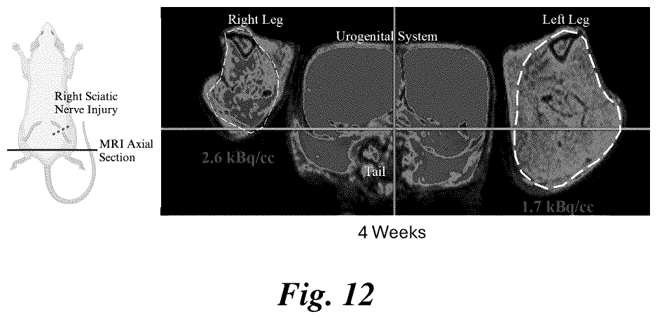

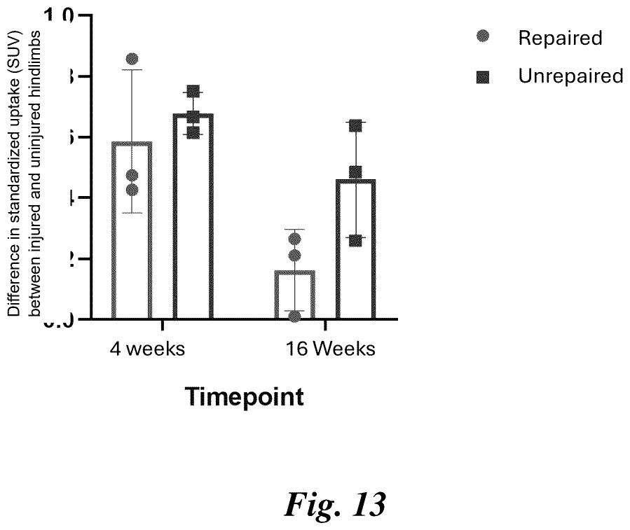

Six adult Lewis rats underwent serial ⁶⁸Ga-PSMA-11 PET-MRI (right sciatic transection with or without repair; n=3 each) at 4 weeks and 16 weeks post-injury. Animals received ⁶⁸Ga-PSMA-11 intravenously 1 hour prior to 7T PET-MRI (Bruker BioSpec 70/30). An example axial slice taken from an animal 4 weeks after unilateral sciatic transection without repair is demonstrated in FIG. 12. At 4 weeks post-injury, the injured limbs in both operative groups had approximately 2 times greater uptake than the uninjured contralateral side (FIG. 13). At 16 weeks post-injury, uptake remained elevated in unrepaired animals at 1.99±0.42 times greater than contralateral limbs, but decreased in repaired animals to 1.33±0.29 times greater than contralateral (FIG. 13).

### Large Animal ⁶⁸Ga-PSMA-11 PET-CT

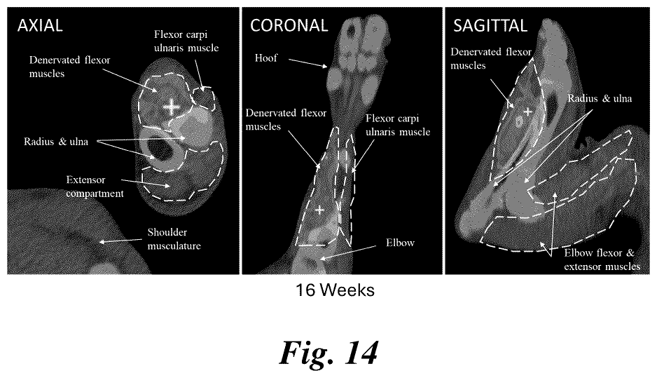

A female Yorkshire pig at age 5 months underwent proximal right median nerve transection with repair and left median nerve transection without repair at the proximal humerus, 12 cm from the olecranon. At 16-weeks post-injury, the animal received intravenous injection of 2.41 mCi of ⁶⁸Ga-PSMA-11 prior to whole-body imaging on a clinical PET-CT scanner (Siemens Biography mCT PET-CT). SUVₘₐₓ (maximum standardized uptake value) was elevated in all median-nerve innervated muscle groups relative to uninjured muscle references (Table 1, FIG. 14). When pooled across muscle groups, denervated muscles had 70% greater SUVₘₐₓ than uninjured, non-denervated muscles (1.39 kBq/cc versus 0.82 kBq/cc).

TABLE 1
Tissue ⁶⁸Ga-PSMA-11 PET-CT uptake
in a swine model for nerve injury.

| Tissue | Averaged (kBq/cc) | SD (kB/cc) |
|---|---|---|
| Right deep flexor compartment | 0.684119 | 0.148651 |
| Left deep flexor comparmtent | 0.767523 | 0.162857 |
| Right superficial flexor | 0.510268 | 0.154572 |
| compartment | | |
| Left superficial flexor compartment | 0.591223 | 0.149287 |
| Forearm Extensor compartments | 0.388471 | 0.098278 |
| Biceps Muscles | 0.416186 | 0.075396 |
| Pectoralis Major muscles | 0.36479 | 0.115247 |
| Latissimus muscles | 0.204879 | 0.075032 |
| Liver | 0.676448 | 0.219343 |

### GCPII Expression is Partly Attributable to Muscle Macrophages

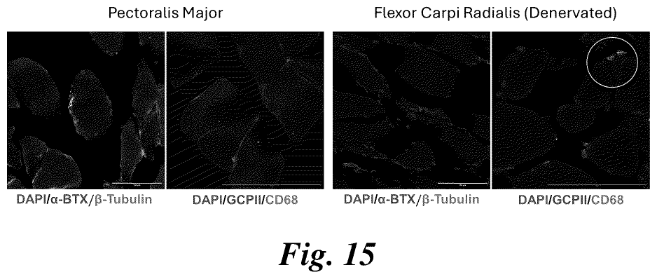

Immunohistochemical analyses were performed on muscles harvested after pig ⁶⁸Ga-PSMA-11 PET-CT. Increased GCPII expression in denervated muscles, such as the flexor carpi radialis, muscle was noted in part to co-localize with CD68, which is a common marker for macrophages (FIG. 15). Note absence of GCPII/CD68 co-localization in the uninjured, non-denervated pectoralis major muscle. The innervation status of both muscles was confirmed by staining for beta tubulin, a common marker for axons, and alpha bungarotoxin, a common marker for neuromuscular junctions.

### Example 3

### Glutamate Carboxypeptidase II (GCPII)-Targeted PET to Identify Muscle Denervation in Peripheral Nervous System Injuries

This Example demonstrates that positron emission tomography (PET) targeting muscle glutamate carboxypeptidase II (GCPII) expression can identify denervation and reinnervation after nerve injury and repair.

### Overview

Management of peripheral nervous system (PNS) neuropathies, including traumatic peripheral nerve injury (PNI), relies on accurate assessment of muscle denervation and recovery. Practical limitations in needle electromyography (EMG), the current gold-standard clinical test, impair diagnostic accuracy and downgrade patient outcomes. We sought to improve the noninvasive characterization of PNIs by using positron emission tomography (PET). We investigated the application of two, clinically-available glutamate carboxypeptidase II (GCPII)-targeted PET agents developed for prostate cancer imaging, e.g., [¹⁸F]DCFPyL and [⁶⁸Ga]PSMA-11, to diagnose muscle denervation and subsequent reinnervation. In preclinical small and large animal PNI models, we found that denervated muscles had approximately twice the uptake of innervated muscles on rat ex vivo biodistribution, rat PET/MRI, and pig PET/CT that persisted for at least 16 weeks post-injury. Importantly, GCPII-targeted uptake resolved with muscle reinnervation by regenerating axons in the setting of surgically repaired PNI. To assess clinical feasibility, we performed [¹⁸F]DCFPyL PET/CT in a patient who had sustained a unilateral radial nerve injury 15 weeks prior and observed elevations in denervated muscle uptake that mirrored our findings in preclinical injury models. The consistency in the magnitude of GCPII-targeted PET uptake across species, its persistence in chronically denervated muscle several months after injury and resolution with muscle reinnervation, and the established safety profile of available PET agents, support GCPII-targeted PET as a highly translatable strategy for characterization and longitudinal monitoring of PNS neuropathies.

### BACKGROUND

Peripheral nervous system (PNS) neuropathies often manifest as persistent and debilitating paralysis from loss of skeletal muscle innervation. Novak et al., 2011. Determining the extent and distribution of muscle denervation is necessary for establishing a correct diagnosis, localizing PNS lesions, and monitoring disease progression or resolution to thereby determine the appropriate treatment strategy. In the case of traumatic peripheral nerve injury (PNI), accurate diagnosis of muscle denervation and subsequent reinnervation is particularly important for guiding timely surgical treatment, given that muscle denervation results in progressive atrophy that diminishes the potential for motor functional recovery. Houdek and Shin, 2015.

Needle electromyography (EMG), the current gold-standard clinical test for muscle denervation, Preston and Shapiro, 2002; Simon et al., 2016, suffers from several important limitations. EMG involves percutaneous placement of electrodes into specific muscles of interest based on surface landmarks, which can be inaccurate when testing small confluent or overlapping muscle bellies or high-risk when testing muscles near critical structures (e.g., intercostal muscles overlying parietal pleura). Cushman et al., 2020; Goodmurphy et al., 2007. Assessment of volitional activity, which is an important component of EMG, relies on patient cooperation, which can be influenced by anxiety, discomfort from needlesticks, or lack of understanding. London et al., 2014; London, 2017; Strommen and Daube, 2001; Daube and Rubin, 2009.

Indeed, because needle placement is painful and testing is time-intensive, it would be impractical for the tester and intolerable for the patient to evaluate all potentially involved muscles. Jan et al., 1999. Therefore, only a subset of muscles is typically selected for testing, which introduces sampling errors that can obscure diagnoses, particularly in the setting of multifocal or proximal PNS lesions. Schreiber et al., 2014; Mackin et al., 1999. Variability in testing equipment and conditions also affect voltage measurements and complicate direct comparison between labs. Fuglsang-Frederiksen and Pugdahl, 2011. Importantly, the interpretations obtained from EMG are largely subjective, highly operator-dependent, and cannot be independently corroborated once testing is complete. Chouteau et al., 2010. Consequently, patients referred for nerve reconstruction often require repeat testing by a specific neurologist or lab that the nerve surgeon trusts, which imposes additional costs, inconvenience and pain for patients, and potentially delays time-sensitive treatment.

### Scope

We sought to address these limitations by using positron emission tomography (PET) to characterize muscle denervation. Specifically, we investigated whether PET imaging with clinically available GCPII-targeted agents could identify muscle denervation and subsequent reinnervation after nerve injury and repair. GCPII, also termed prostate-specific membrane antigen (PSMA), is an established biomarker for prostate cancer. O'Keefe et al., 2018. Multiple GCPII-targeted PET agents are approved in the United States and Europe for prostate cancer imaging, such as [⁶⁸Ga]PSMA-11 and [¹⁸F]DCFPyL. Bois et al., 2020; Pienta et al., 2021. Although GCPII is expressed in several tissues outside of the prostate, Barinka et al., 2012; Ismail et al., 2023, its activity is typically very low in normal adult skeletal muscle. Jansen et al., 2019; Rovenski et al., 2008; Foss et al., 2005; Ferreira et al., 2019. GCPII inhibition, however, was recently shown to delay muscle loss in preclinical models for amyotrophic lateral sclerosis (ALS) and age-related atrophy, both of which involve muscle denervation. Tallon et al., 2022; Su et al., 2024; Horwath, 2025. We therefore hypothesized that uptake of GCPII-targeted imaging agents may be elevated in denervated muscles after PNI.

In this Example, we first examined GCPII expression at multiple timepoints within both persistently denervated and reinnervated muscle using near-infrared (NIR) and PET imaging in rat PNI models. To assess translatability, we next performed [⁶⁸Ga]PSMA-11 PET/CT in a pig median nerve transection model. Finally, we performed [¹⁸F]DCFPyL PET/CT in a patient 15 weeks after sustaining a complete proximal radial nerve injury. Our findings demonstrate the effectiveness of GCPII-targeted PET in diagnosing muscle denervation and subsequent reinnervation, offering the promise of an objective and painless test that addresses the critical limitations of EMG for patients with traumatic PNI and other PNS neuropathies.

### Results

### Muscle GCPII Expression Localizes in Part to the Perinuclear Sarcolemma

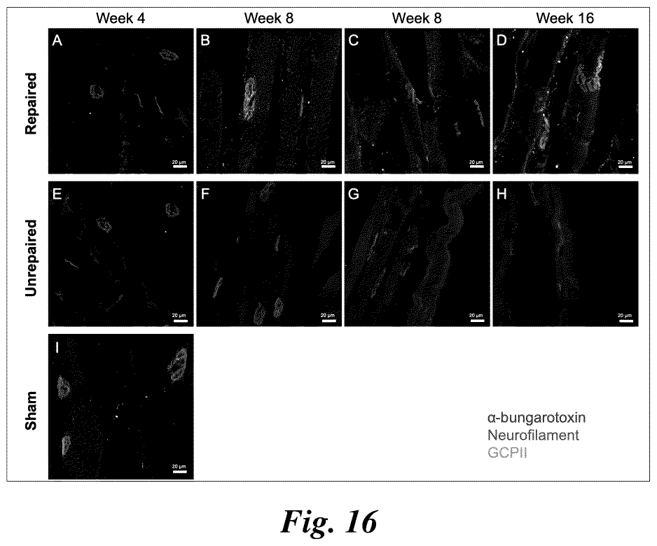

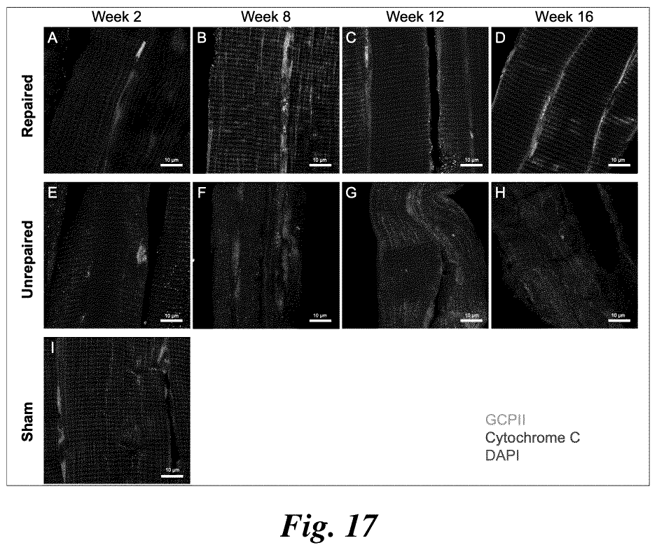

Muscle GCPII expression was evaluated in denervated and reinnervating muscles at multiple timepoints post-injury. Lewis rat hindlimb muscles were subjected to either sustained denervation (i.e., nerve transection without repair) or to denervation with subsequent reinnervation (i.e., nerve transection with immediate reconstruction). Reinnervation began between four and eight weeks after nerve transection with repair (FIG. 16 panels A-B) and continued until complete reinnervation of NMJs by 16 weeks (FIG. 16 panel D). By contrast, NMJs remained denervated and progressively fragmented by 16 weeks in animals that underwent nerve transection without repair, confirming that persistent muscle denervation was achieved (FIG. 16 panels E-H). Clusters of GCPII expression were identified around intact and reinnervated NMJs in the sham and repaired animals, respectively (FIG. 16 panels B-D, panel I), but not around denervated NMJs in the unrepaired animals or early repaired animals (FIG. 16 panel A, panels E-H). Importantly, GCPII expression at innervated NMJs accounted for a minority of GCPII expression in the overall samples. Instead, in both innervated and denervated muscles, GCPII expression primarily localized to the sarcolemma adjacent to clusters of perinuclear subsarcolemmal mitochondria (FIG. 17). These perinuclear clusters persisted over time in denervated muscles (FIG. 17 panels E-H) despite the declines in mitochondrial content that are expected from denervation-induced mitophagy. Yang et al., 2020.

Near-Infrared (NIR) Imaging with YC27 Identifies Denervated and Reinnervated Muscles after Nerve Injury

We next assessed whether uptake of GCPII-targeted agents varied with muscle denervation and reinnervation. We first evaluated muscle uptake of YC27, a fluorescent compound that specifically binds to GCPII and emits in the near-infrared region, Chen et al., 2009, as NIR imaging allows for higher spatial resolution than PET. We generated diverse injuries to the sciatic nerve and its distal branches, both with and without nerve repair, in Lewis rats and performed NIR imaging at multiple timepoints post-injury. This assessment was done to examine the selectivity of YC27 uptake for specific muscles or muscle groups, its persistence over time in chronically denervated muscles in the absence of nerve repair, and its responsiveness to varying degrees of muscle reinnervation by regenerating axons in the setting of nerve repair. The rat sciatic nerve branches into the tibial nerve to innervate posterior compartment leg muscle, such as the medial gastrocnemius muscle, and the common peroneal nerve to innervate anterior compartment leg muscles, such as the tibialis anterior muscle. Whereas a sciatic nerve transection with repair results in at least four weeks of denervation in the anterior and posterior compartment muscles, Menovsky and Beek, 2001; Tang et al., 2019; Hundepool et al., 2018; English et al., 2007; Dinh et al., 2009; Saltzman et al., 2019; Penna et al., 2012; Ijkema-Paassen et al., 2002, a common peroneal nerve crush injury results in two weeks of denervation in anterior compartment only followed by more rapid muscle reinnervation by axons regenerating across the low-grade injury site. Aydin et al., 2006; Swett et al., 1991; Fontaine et al., 2021; Farinas et al., 2020.

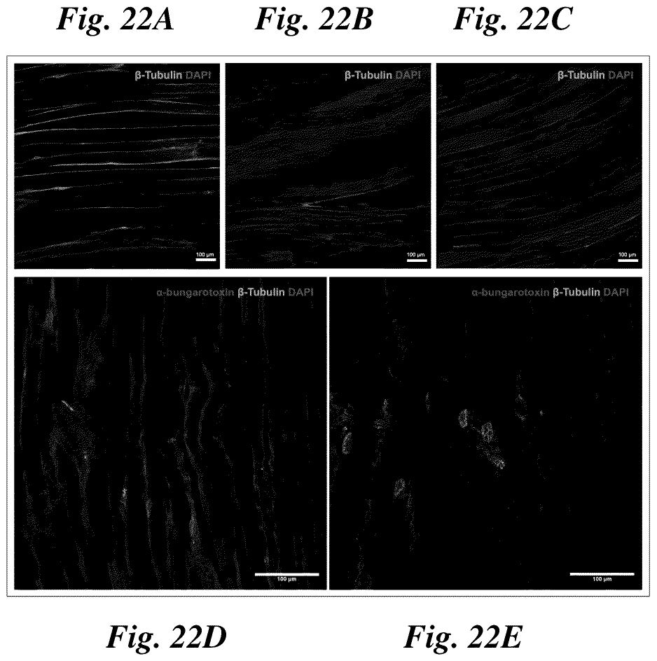

Accordingly, YC27 uptake was found to be elevated within denervated muscles groups two weeks after nerve transection without repair, nerve transection with repair, and nerve crush, with highly specific uptake noted following selective transection of distal muscle branches (FIG. 18 panels A-C). At four weeks, YC27 uptake remained elevated in affected muscles after nerve transection irrespective of nerve repair (FIG. 18 panel D, panel E), whereas uptake resolved in anterior compartment muscles after common peroneal nerve crush (FIG. 18 panel F). As anticipated, histological assessment of affected muscles at four weeks post-injury confirmed reinnervation after common peroneal nerve crush and continued denervation after sciatic nerve transection irrespective of repair (FIG. 22). By 16 weeks after sciatic nerve transection, YC27 uptake remained elevated only in the setting of nerve transection without repair (FIG. 18 panel G, FIG. 18 panel H). See also FIG. 7, FIG. 8, and FIG. 9.

### [¹⁸F]DCFPyL Uptake is Elevated in Denervated Muscles

To quantify GCPII-targeted agent uptake within denervated and reinnervated muscles, 48 rats (24 male, 24 female) underwent unilateral sciatic nerve transection without repair, sciatic nerve transection with repair, or sham surgery. At two, four, or 16 weeks post-injury (n=16 per timepoint), animals received 5.18±1.67 MBq of [¹⁸F]DCFPyL intravenously prior to gastrocnemius muscle harvest. Tissue radioactivity was expressed as percent injected dose per gram (% ID/g). Gastrocnemius muscles in both repaired and unrepaired groups demonstrated increased uptake relative to sham at two weeks (mean % ID/g: repaired 0.26, unrepaired 0.23, sham 0.13) and four weeks (mean % ID/g: repaired 0.31, unrepaired 0.37, sham 0.073) (FIG. 10). At 16 weeks, elevated [¹⁸F]DCFPyL uptake persisted in unrepaired animals (mean % ID/g: 0.36) when compared to sham (mean % ID/g: 0.15), but uptake resolved toward sham in repaired animals (mean % ID/g: 0.19). When pooled across timepoints, mean gastrocnemius muscle % ID/g in unrepaired groups was 0.32±0.13 compared to 0.12±0.06 for sham (p<0.0001).

### Muscle [¹⁸F]DCFPyL Uptake is Attributable to Changes in GCPII Expression

To confirm that [¹⁸F]DCFPyL uptake in denervated muscles is specific for GCPII expression, four additional animals underwent co-injection of 3.7±0.074 MBq of [¹⁸F]DCFPyL and 100 mg/kg of ZJ-43, a potent competitive inhibitor of GCPII binding, Yamamoto et al., 2004, four weeks after sciatic nerve transection without repair. ZJ-43 administration eliminated differences in [¹⁸F]DCFPyL uptake between denervated and non-denervated gastrocnemius muscles (FIG. 11).

Sustained [⁶⁸Ga]PSMA-11 Uptake within Denervated Muscles on Serial Rodent PET/MRI

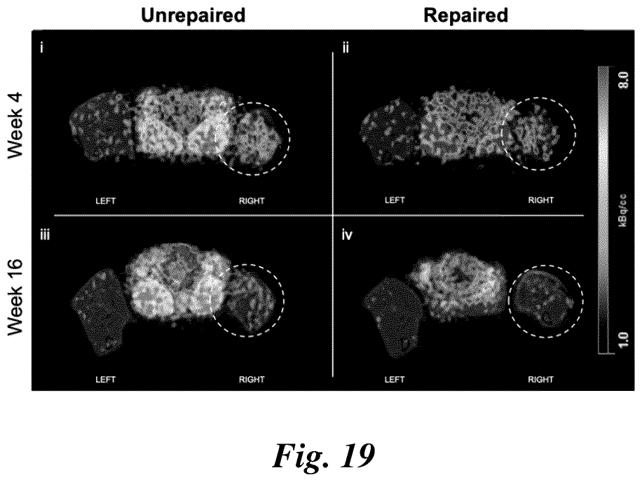

To assess whether changes in muscle GCPII uptake are detectable on PET, we next performed serial [⁶⁸Ga]PSMA-11 PET-MR in six male Lewis rats after right sciatic nerve transection with or without repair (n=3 per group). Animals were imaged at four weeks and 16 weeks post-injury. Consistent with [¹⁸F]DCFPyL biodistribution results, both groups exhibited increased uptake at four weeks (FIG. 19A); the uptake ratio in affected versus unaffected hindlimbs was 2.12±0.14 and 2.29±0.15 for repaired and unrepaired groups, respectively (FIG. 13). At 16 weeks, uptake ratio remained elevated in unrepaired animals at 1.85±0.18, whereas it declined to 1.37±0.17 in repaired animals (p=0.037).

Pig [⁶⁸Ga]PSMA-11 PET/CT Identifies Denervated Muscles after Median Nerve Injury

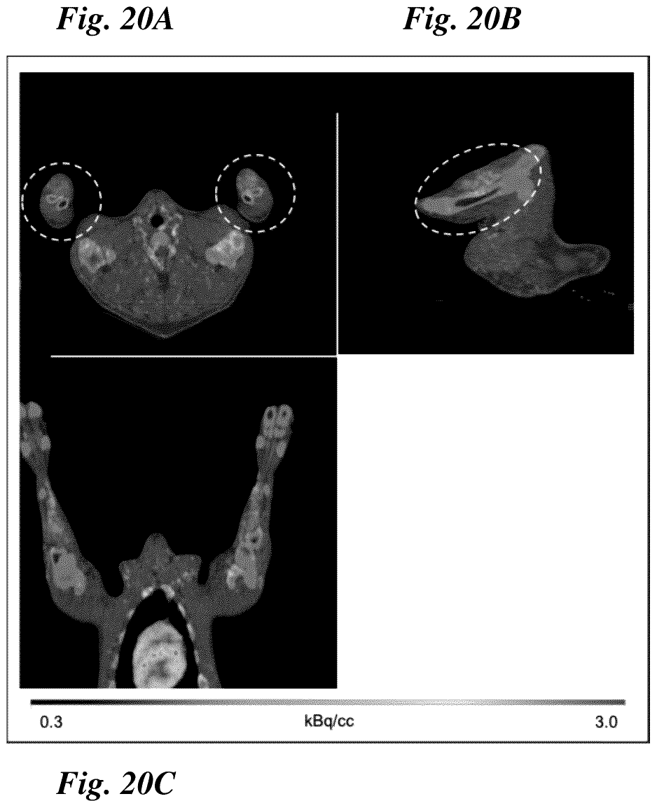

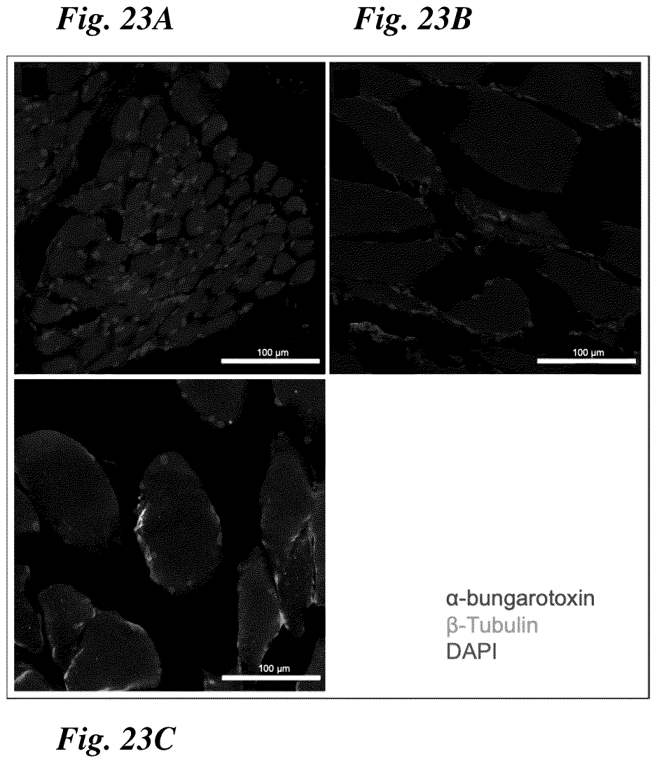

To study the translatability of our rodent PET-MR findings in a large animal pre-clinical model, a female Yorkshire pig underwent proximal right median nerve transection with repair and left median nerve transection without repair at the proximal humerus. The median nerve diameter, regenerative capacity, and regenerative distance between injury and forearm flexor muscles are comparable to humans. Scholz et al., 2010; Smith et al., 2022. At 16 weeks post-injury, the animal received 89.17 MBq of [⁶⁸Ga]PSMA-11 prior to whole-body PET/CT. A 16-week timepoint was selected to allow for direct comparison against rat PET/MRI results. Mean activity was elevated in all median-nerve innervated muscle groups relative to uninjured muscle references (Table 2, FIG. 20). When pooled across muscle groups, denervated muscles had 1.88 times greater activity than uninjured muscles (0.64 kBq/cc versus 0.34 kBq/cc). Bilateral median-nerve innervated muscles remained denervated at this timepoint irrespective of repair (FIG. 23).

TABLE 2
Tissue [⁶⁸Ga]PSMA-11 uptake on pig PET/CT.

| Tissue | Status | Mean Activity (kBq/cc) |
|---|---|---|
| Deep Flexor Compartment | | |
| Left | Injured | 0.77 ± 0.16 |
| Right | Injured | 0.68 ± 0.15 |
| Superficial | | |
| Flexor Compartment | | |
| Left | Injured | 0.59 ± 0.15 |
| Right | Injured | 0.51 ± 0.15 |
| Forearm Extensor | Unaffected | 0.39 ± 0.098 |
| Compartments | | |
| Biceps Muscles | Unaffected | 0.42 ± 0.075 |
| Pectoralis Major Muscles | Unaffected | 0.36 ± 0.12 |
| Latissimus Muscles | Unaffected | 0.20 ± 0.075 |
| Liver | Reference | 0.68 ± 0.22 |
| | organ | |

Table 2. Tissue [⁶⁸Ga]PSMA-11 uptake on pig PET/CT. Both superficial and deep flexor compartments are affected by a median nerve injury. Note lower uptake in unaffected muscle groups. Mean activity within each muscle or compartment of muscles is expressed in kilobecquerel per cc (kBq/cc).

Human [¹⁸F]DCFPyL PET/CT Identifies Denervated Muscles after Radial Nerve Injury

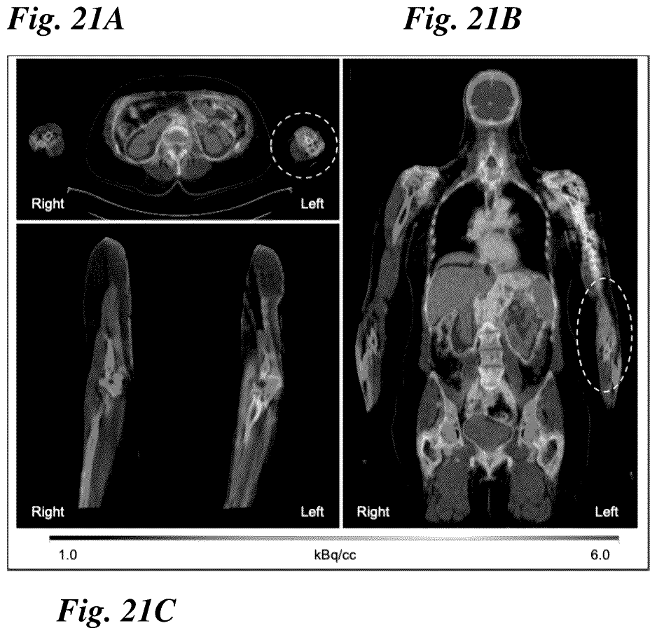

Following validation in small and large animals, we performed [¹⁸F]DCFPyL PET/CT in a 55-year-old woman who had sustained a complete left radial nerve palsy 15 weeks prior. This patient was selected for her discrete, clinically well-defined nerve injury with a similar chronicity as our preclinical injury models. Physical examination and electrodiagnostic evaluation prior to imaging confirmed absent radial nerve function and complete muscle denervation distal to the left triceps muscle, including the supinator and brachioradialis muscles. The patient received 344.1 MBq of [¹⁸F]DCFPyL one hour prior to lose-dose CT from vertex of the head to mid-thigh, with two minutes of PET acquisition per field-of-view. Consistent with our rat and pig PET results, activity in affected left radial nerve-innervated muscles was 2.0 times greater than the contralateral arm (Table 3, FIG. 21).

TABLE 3
Tissue [¹⁸F]DCFPyL uptake on human PET/CT.

| Tissue | Status | Mean Activity (kBq/cc) |
|---|---|---|
| Mobile Wad Compartment | | |
| Left | Injured | 2.17 ± 0.24 |
| Right | Unaffected | 0.96 ± 0.12 |
| Forearm Extensor | | |
| Compartment | | |
| Left | Injured | 2.17 ± 0.35 |
| Right | Unaffected | 1.21 ± 0.13 |
| Forearm Flexor | | |
| Compartments | | |
| Left | Unaffected | 1.27 ± 0.13 |
| Right | Unaffected | 1.08 ± 0.15 |
| Triceps Muscle | | |
| Left | Unaffected | 1.16 ± 0.20 |
| Right | Unaffected | 0.94 ± 0.18 |
| Pectoralis Major Muscle | | |
| Left | Unaffected | 1.09 ± 0.14 |
| Right | Unaffected | 0.83 ± 0.14 |
| Thigh Anterior | | |
| Compartment | | |
| Left | Unaffected | 0.84 ± 0.15 |
| Right | Unaffected | 0.87 ± 0.18 |
| Thigh Posterior | | |
| Compartment | | |
| Left | Unaffected | 0.76 ± 0.21 |
| Right | Unaffected | 0.81 ± 0.19 |
| Liver | Reference organ | 9.98 ± 0.71 |
| Kidney | Reference organ | 62.65 ± 9.75 |

Table 3: Tissue [¹⁸F]DCFPyL uptake on human PET/CT. The mobile wad compartment consists of the radial nerve-innervated brachioradialis, flexor carpi radials longus, and flexor carpi radials brevis muscles. The forearm extensor compartment consists of wrist and digital extensor muscles. The triceps muscle, while innervated by the radial nerve, receives innervation proximal to the site of injury. Note increased uptake in injured muscle groups when compared to unaffected muscles, including the left triceps muscle. Mean activity within each muscle or compartment of muscles is expressed in kilobecquerel per cc (kBq/cc).

### DISCUSSION

A key finding of this Example is that GCPJJ-targeted PET agents can identify muscle denervation and subsequent reinnervation after nerve injury and repair. We observed that both innervated and denervated myocytes express GCPII, predominantly in the perinuclear sarcolemma near clusters of subsarcolemmal mitochondria. Denervation increased muscle uptake of the NIR agent YC27 and the PET agents [¹⁸F]DCFPyL, and [⁶⁸Ga]PSMA-11 at all tested timepoints between two and 16 weeks post-injury. Similarly, muscle reinnervation led to a reduction in uptake of YC27, [¹⁸F]DCFPyL, [⁶⁸Ga]PSMA-11 toward baseline. We also demonstrated that the same [¹⁸F]DCFPyL PET protocol (i.e., dose, time interval between injection and imaging, and acquisition time) commonly used for prostate cancer imaging could accurately identify the pattern of muscle denervation in a woman who had sustained a closed radial nerve injury.

GCPII converts N-acetyl-aspartyl-glutamate (NAAG) into glutamate and N-acetyl-aspartate (NAA) and also releases glutamate from pteroylpoly-gamma-glutamate to form pteroylglutamate (i.e., folate). Barinka et al., 2002. It is well known that denervation increases myocyte intracellular glutamate concentrations. Rennie et al., 1989; Turinsky and Long, 1990. The consequences of this increase, and importance of GCPII in these processes, however, are not well defined. The role of GCPII within prostate cancers is similarly poorly understood despite the widespread use of GCPII-targeted PET for prostate cancer diagnosis. Roberts et al., 2023. Glutamate signaling modulates competitive synaptic elimination at mammalian NMJs during postnatal development, and GCPII is expressed at NMJs during this period. Marmiroli et al., 2012; Personius et al., 2016; Personius et al., 2022.

While polyneuronal innervation and synaptic elimination also has been demonstrated at nascent NMJs in reinnervating adult rat muscle, Favero et al., 2007, our histology demonstrated that GCPII was only expressed at innervated NMJs within sham and reinnervated muscles. NMJs also accounted for relatively little GCPII staining, and NMJ GCPII expression would not explain the uniform increases in GCPII-targeted agent uptake seen within denervated muscles. Similar findings were reported in a rodent model for amyotrophic lateral sclerosis (ALS), in which the vast majority of muscle GCPII activity was unexpectedly expressed outside of NMJs. Tallon et al., 2022.

Co-localization of GCPII with subsarcolemmal nuclei suggests a possible link to mitochondrial reactive oxygen species (ROS) generation, which are important modulators of denervation-induced autophagy. Adhihetty et al., 2007; Muller et al., 2007; O'Leary et al., 2012. The relationship between glutamate release and oxidative stress is well described in neurodegenerative disorders, Coyle and Puttfarcken, 1993, and inhibition of GCPII has been shown to be neuroprotective in several preclinical brain injury models. Cao et al., 2016; Rahn et al., 2012. Although glutamate influences ROS production in non-synaptic brain mitochondria, Panov et al., 2009; Selivanov, 2021, the significance of this process in denervated muscles is unknown. Alternatively, increased GCPII expression may reflect a compensatory change from broader alterations in protein degradation and synthesis. Argadine et al., 2009. For example, denervation causes constitutional activation of both the ubiquitin-proteasome pathway for protein degradation and rapamycin complex 1 (mTORC1), a primary suppressor of autophagy. Kim and Guan, 2015; Quy et al., 2013. Glutamate can act as an intracellular reservoir for glutamine, which is important for several anabolic and antiapoptotic pathways including mTORC1 modulation, potentially explaining the perinuclear localization of GCPII in this Example. Jewell et al., 2015; Brasse-Lagnel et al., 2009; Nguyen et al., 2019. A link between GCPII expression and constitutional changes in protein homeostasis also is consistent with the sustained elevation in GCPII-targeted agent uptake observed across timepoints.

GCPII-targeted PET addresses several limitations of EMG by providing quantitative, reproducible, and noninvasive evaluation of all potentially affected muscles, generating imaging data that can be independently interpreted and verified by treating physicians. Jan et al., 1999; Fuglsang-Frederiksen and Pugdahl, 2011. Our findings also support the superiority of GCPII-targeted PET to other previously investigated PET-based approaches for this indication. Specifically, fluorodeoxyglucose (FDG)-PET has demonstrated poor efficacy for identifying muscle denervation in patients with PNIs. Parida et al., 2017; Choi et al., 2019. This observation is unsurprising as changes in muscle glucose metabolism after nerve injury are heterogeneous and short-lived, with utilization actually increasing after denervation in some cases. Pak et al., 2016; Lee et al., 2014; Lee et al., 2019; Nam et al., 2019. By contrast, GCPII-targeted PET demonstrated consistent differences between denervated and non-denervated muscles that were sustained over several months following PNI without repair. GCPII-targeted PET has special relevance to the management of PNS neuropathies that produce complex and unpredictable patterns of muscle denervation, such as proximal PNI involving the brachial plexus or lumbosacral plexus, multi-focal PNIs and radiculopathies, Parsonage Turner syndrome (i.e., neuralgic amyotrophy), and many other motor neuron pathologies. In such cases, comprehensive evaluation of all potentially involved muscle groups will avoid the risk of sampling errors inherent in EMG. O'shea et al., 2011. GCPII-targeted PET also has the potential to improve our ability to diagnose muscle denervation resulting from concurrent lower motor neuron (LMN) injury in the setting of spinal cord injury (SCI). Approximately 80% of cervical SCIs involve damage to cell bodies of PNS motor neurons that reside in the ventral horn. Mulcahey et al., 1999; Berger et al., 2023. EMG is poorly suited to diagnose muscle denervation in this setting of SCI, as patients are unable to volitionally recruit motor unit potentials regardless of whether LMN injury is present. Indeed, EMG is particularly unreliable in this population. Jain et al., 2020. With the advent of nerve transfers to restore upper extremity function following cervical SCI, there is an acute need to establish reliable methods of diagnosing concurrent LMN injury to determine candidacy and timing for these procedures. Berger et al., 2022; Fox et al., 2015; Fox et al., 2018; Hill and Fox, 2019; Javeed et al., 2022; van Zyl et al., 2019; Khalifeh et al., 2019.

This Example has several limitations. For one, the persistence of GCPII-targeted PET uptake in the setting of very long-term denervation remains unclear. Although the histologic and functional consequences of chronic denervation-induced muscle atrophy in rodents are analogous to those in humans, the rate of progression is accelerated in rats, with end-stage muscle atrophy reached with approximately seven months of denervation compared to 18-24 months in humans. Sakuma et al., 2016; Sarhane et al., 2021.

We evaluated muscles at 16 weeks post-injury as it approximates the end of the clinically-relevant window in patients, beyond which functional muscle reinnervation becomes unlikely. Gordon et al., 2003; Gordon et al., 2011. While our clinical PET data from a patient with PNI provides a promising proof-of-concept, additional clinical testing is needed to define the clinical sensitivity and specificity of this diagnostic modality. In particular, while GCPII-targeted PET provides quantitative data that may enable characterization of varying degrees of partial denervation, additional studies will be needed to investigate this question. Although beyond the scope of this work, mechanistic investigations to elucidate the pathophysiologic underpinnings and implications of GCPII activity in denervated muscle will shed light on potential therapeutic targets to mitigate the deleterious effects of denervation-induced muscle atrophy.

In summary, GCPII-targeted PET is a promising method to diagnose muscle denervation, addressing several unmet needs in the evaluation of PNS insults. Given the established safety and clinical availability of [¹⁸F]DCFPyL and [⁶⁸Ga]PSMA-11, GCPII-targeted PET is well-positioned for rapid translation to patient care.

### MATERIALS AND METHODS

### Study Design

An objective of this Example was to address the unmet need for noninvasive diagnosis and monitoring of complex PNS neuropathies. To address this objective, we evaluated whether muscle uptake of GCPII-targeted PET agents could serve as a noninvasive biomarker for muscle denervation. While several proteins are elevated in denervated muscles and traumatized nerves, GCPII is a highly attractive target because GCPII-based PET is already employed clinically for prostate cancer imaging. Without wishing to be bound to any one particular theory, it is thought that the PET agents, [¹⁸F]DCFPyL and [⁶⁸Ga]PSMA-11, could be repurposed to characterize muscle innervation. We evaluated the uptake of known GCPII-targeted agents in small- and large-animal models after various nerve injuries and at multiple timepoints post-injury. We first performed ex vivo NIR imaging in nerve-injured Lewis rats after intravenous YC27 injection. We corroborated our NIR results using a separate cohort of rats, which underwent unilateral sciatic transection without repair, transection with repair, or sham surgery. At two, four, or 16 weeks post-injury, these animals received intravenous [¹⁸F]DCFPyL injection prior to tissue harvest and ex vivo quantification using an automated gamma counter. A third cohort of rats underwent serial in vivo [⁶⁸Ga]PSMA-11 PET/MRI at four and 16 weeks after sciatic nerve transection with or without repair. We next examined the translatability of these results to large animals by performing [⁶⁸Ga]PSMA-11 PET/CT in a Yorkshire pig 16 weeks after bilateral median nerve injuries. Finally, we evaluated muscle [¹⁸F]DCFPyL PET/CT uptake in a 55-year-old woman who had sustained a left radial nerve injury 15 weeks prior. For these experiments, histologic assessments were blinded to injury model. Uptake measurements of GCPII-targeted agents were unblinded, as the injuries produced overt anatomic changes to affected limbs. Animal protocols were approved by our Institutional Animal Care and Use Committee (Protocols RA21M203, RA22M257, SW23M91) and were conducted per National Institutes of Health (NIH) guidelines for animal welfare. Clinical imaging was performed after obtaining informed consent and with Institutional Review Board approval (IRB00422704).

### Synthesis of GCPII-Targeting Imaging Agents

The properties and synthesis of YC27 were described previously. Chen et al., 2009. In brief, YC27 is a high-affinity GCPII-binding urea (Kᵢ 0.37 nM) that is functionalized with the commercially-available NIR dye, IRDye800CW (LI-COR Biosciences, Lincoln, NE, USA). YC27 exhibits a maximum absorbance of 774 nm and fluoresces with a maximum emission at 792 nm. For animal studies, [¹⁸F]DCFPyL was synthesized at the Johns Hopkins PET Center as described previously. Chen et al., 2011. [⁶⁸Ga]PSMA-11 (Illuxix®; Telix Pharmaceuticals, Melbourne, Australia) was purchased from a local radiopharmacy and supplied in a 10 mL solution suitable for injection. For both radiopharmaceuticals, 100-300 μL doses of the parent solution were drawn to reach the target activity necessary for rodent and pig evaluations. For the clinical scan, [¹⁸F]DCFPyL was purchased from the clinical manufacturer (Pylarify®; Lantheus, North Billerica, MA, USA).

### Animal Models

Rodent surgical procedures involved Lewis rats (Charles River, MD, USA) aged 14-16 weeks and were performed under general anesthesia via inhalational isoflurane. The rat sciatic nerve branches into a common peroneal nerve branch to anterior leg compartment muscles and a tibial nerve branch to posterior leg compartment muscles, including separate branches to the medial and lateral heads of the gastrocnemius muscle. For animals undergoing nerve transection with repair, the sciatic nerve was sharply divided using straight microsurgical scissors and immediately repaired using three 8-0 non-absorbable monofilament epineurial sutures. For sciatic nerve transection without repair, the nerve was sharply transected, and the distal stump was mobilized upward 120 degrees clockwise and tacked to nearby muscle fascia using one 8-0 non-absorbable monofilament suture to prevent spontaneous reinnervation. For common peroneal nerve crush, the common peroneal nerve was exposed at its origin from the sciatic nerve and a Webster 5″ smooth needle holder (Aesculap, MO, USA) was closed over the nerve for 30 seconds. For isolated medial head of gastrocnemius muscle denervation, the tibial nerve was identified at its origin at the sciatic nerve and exposed distally between the medial and lateral heads of the gastrocnemius muscles. The branch to the medial head was isolated and segmentally resected from its origin at the tibial nerve to its muscular insertion to prevent spontaneous reinnervation. For sham surgery, the sciatic nerve was exposed and gently mobilized from the underlying muscle fascia. In all rodents, the surgical wound was closed in layers using 4-0 absorbable sutures.

The pig surgery involved a female Yorkshire pig (Archer Farms, MD, USA) aged 22 weeks. In a dedicated large animal operating room, the animal was placed supine with bilateral upper extremities abducted 90 degrees. General anesthesia was provided via endotracheal isoflurane. For each upper extremity, a longitudinal incision was made in the axilla just posterior to the anterior axillary fold. The pectorals major muscle belly was bluntly split longitudinally to expose the proximal median nerve, which was sharply transected using an 11-blade scalpel at a position 10 cm from the sternum and 12 cm from the olecranon. On the right extremity, the nerve was immediately repaired using six interrupted 8-0 non-absorbable monofilament epineurial sutures. On the left extremity, the nerve was left in discontinuity, and the distal stump was mobilized upward 120 degrees and tacked to the pectoralis major muscle fascia using two 8-0 non-absorbable monofilament sutures to prevent spontaneous reinnervation. The pectoralis muscle was repaired, and the skin closed in layers using 4-0 absorbable sutures.

### Ex Vivo Biodistribution

Forty-eight rats (24 male, 24 female) received right sciatic nerve transection without repair (n=18), transection with repair (n=15), or sham surgery (n=15), and underwent ex vivo biodistribution studies at two, four, or 16 weeks post-injury (n=16 per timepoint). Animals received 5.18±1.67 MBq of [¹⁸F]DCFPyL via tail vein injection two hours prior to euthanasia by isoflurane overdose and cervical dislocation. Tissues were rapidly harvested and weighed prior to quantification using an LKB CompuGamma CS 1282 automated gamma counter (PerkinElmer, Waltham, MA, USA). Tissues included the right gastrocnemius muscle (operated side), left gastrocnemius muscle (non-operated side), blood, heart, and kidney. Counts were background- and decay-corrected and compared against a 1:10 diluted standard dose to calculate percent injected dose per gram (% ID/g) for each tissue. This calculation was performed as follows, where CPM represents counts per minute:

$$\frac{\% ID}{g} = \frac{1 : 10 Standard Dose ( Bq )}{Injected Dose ( Bq )} \times \frac{Sample CPM - Background CPM}{Standard CPM} \times \frac{1}{Sample Weight ( g )} \times 100 \%$$

### Blocking Studies

An additional four Lewis rats underwent sciatic nerve transection without repair. At four weeks post-injury, animals received co-injection of 3.7±0.74 MBq of [¹⁸F]DCFPyL and 100 mg/kg of the competitive GCPII inhibitor ZJ-43 (Tocris Biosciences, Bristol, UK). Ex vivo biodistribution of [¹⁸F]DCFPyL was assessed after two hours of uptake time as described above.

### Near-Infrared (NIR) Imaging

Six Lewis rats received multiple nerve injuries, including sciatic nerve transection with or without repair, common peroneal nerve crush, and selective transection of the medial gastrocnemius muscle branch of the tibial nerve. Imaging was performed two, four, or 16 weeks post-injury. Animals received a 10-nM dose of YC27 via tail vein injection 24 hours prior to euthanasia, hindlimb disarticulation, and imaging using a Pearl Impulse Near-Infrared Imager (LI-COR Biosciences, Lincoln, NE, USA). Images were acquired with a 790/800 nm band-pass filter and overlaid on a white light photograph using the manufacturer's software (Pearl Impulse version 2.0).

### Small Animal PET-MR

Six rats underwent sciatic nerve transection without repair (n=3) or transection with repair (n=3). Animals were imaged serially using a 7T simultaneous PET/MRI scanner (Bruker BioSpec 70/30; Bruker Corporation, Billerica, MA, USA) at four and 16 weeks post-injury. An average 4.44±1.67 MBq of [⁶⁸Ga]PSMA-11 was administered via tail vein injection one hour prior to each imaging session. Scans were performed from hip to ankle using the following parameters: multislice TurboRARE T2-weighted MRI scan with echo time msec/repetition time msec (TE/TR) of 30/4000, resolution of 0.25×0.25 mm, field of view of 70×50 mm, 40 sections, 1-mm section thickness, matrix size of 280×200, and four averages. Simultaneous 10-minute static PET scans were acquired using a 72-mm PET-optimized Tx/Rx radiofrequency coil (Bruker Corporation, Billerica, MA, USA) centered inside the PET detector. The acquired PET images were reconstructed using a three-dimensional maximum-likelihood expectation-maximization iterative image reconstruction algorithm with a pixel size of 0.5 mm and 18 iterations. The PET-MR images were spatially co-registered using a geometry-based approach with MRI images as a reference in the Bruker ParaVision 360 software and converted to Digital Imaging and Communications in Medicine format (DICOM). The data visualizations and quantitative analysis were performed using PMOD software version 4.004 (PMOD Technologies, Zurich, Switzerland).

### Large Animal PET/CT

At 16 weeks post-injury, the Yorkshire pig underwent intravenous injection of 89.17 MBq of [⁶⁸Ga]PSMA-11 and was imaged on a Siemens Biograph mCT PET/CT scanner (Siemens Healthineers, Knoxville, TN, USA) after one hour of uptake. PET data were collected for 70 minutes using a multi-bed dynamic protocol. The animal was positioned supine and maintained under general anesthesia via endotracheal isoflurane for the duration of the scan. Following imaging, the animal was euthanized via barbiturate overdose. PET images were reconstructed with ordered subsets expectation maximization (OSEM) algorithm, with corrections for attenuation, scatter, random, and time-of-flight. A total of two iterations with 21 subsets per iteration were used. A post-reconstruction Gaussian filtering with a filter size of 5 mm was applied to reduce noise. The software package PMOD version 3.7 (PMOD Technologies, Zurich, Switzerland) was used for PET image processing and analysis. The volumes of interest (VOIs) were manually drawn on co-registered PET/CT images. The mean and standard deviation of tracer activity in each VOI was then quantified from PET images.

### Clinical PET/CT

A 55-year-old female with a left radial nerve injury at the mid-humerus resulting in absent radial nerve function distal to the triceps muscle underwent imaging 15 weeks post-injury. The patient received 345.21 MBq of [¹⁸F]DCFPyL and underwent Siemens Biograph Vision PET/CT (Siemens Healthineers, Knoxville, TN, USA) after one hour of uptake. The scan was performed from the vertex of the head to mid-thigh using seven bed positions, with two minutes of acquisition per bed position. This protocol is the same as is typically used for prostate cancer imaging at our center. PET images were reconstructed with OSEM algorithm, with corrections for attenuation, scatter, random, and time-of-flight. A total of eight iterations with five subsets per iteration were used. A post-reconstruction Gaussian filtering with a filter size of 6 mm was applied. The VOIs were manually drawn on co-registered PET/CT images and quantified using PMOD software as described above.

### Histologic Evaluation

Immunohistochemistry analysis was performed on tissue sections fixed with 4% paraformaldehyde (PFA) and cryoprotected in 15% and 30% sucrose. Tissues were embedded in O.C.T. tissue freezing medium (ThermoFisher 23-730-571) and sectioned at 20-am thickness (HM525 NX, Epredia). Slides were permeabilized with 0.2% Triton X-100 for ten minutes. Sections were blocked for an hour with 5% normal goat serum and 2% bovine serum albumin. Sections were incubated overnight at 4° C. with the primary antibodies of interest (see herein below). After washing three times with PBS, tissue sections were treated on the following day with the secondary antibodies (see herein below). Sections were mounted using Fluoromount G mounting medium with DAPI for nuclear staining. Confocal imaging was performed using a Zeiss LSM 800 laser scanning confocal microscope (Carl Zeiss AG, Oberkochen, Germany) equipped with Plan-Apochromat 20×/0.8 NA and 63×/1.4 NA oil DIC objectives and 405, 488, 561, and 640 nm laser lines. Images were acquired with a pinhole size set to 1 Airy unit, bidirectional scanning, scan speed of 8, and 2× line averaging. For z-stack acquisition, the optimal step size was calculated according to the Nyquist theorem. Maximum intensity projections were generated for three-dimensional visualization. Image acquisition and processing were controlled using ZEN Blue software. For GCPII content, primary antibody was rabbit anti-GCPII polyclonal antibody (Proteintech, 13163-1-AP, 1:50), secondary antibody was anti-rabbit CoraLite488-conjugated IgG (H+L) (Proteintech, SA00013-2, 1:200). For mitochondrial staining, primary antibody was mouse anti-cytochrome C monoclonal antibody (Invitrogen, 45-6100, 1:100), secondary antibody was goat anti-mouse Alexa Fluor 594 IgG1 (Invitrogen, A-21125, 1:200). For motor endplates, we used alpha-bungarotoxin conjugate (Invitrogen, B13423, 1:1000). For neurite infiltration, primary antibodies were chicken anti-neurofilament H (200 kDa) antibody (Millipore Sigma, AB5539, 1:800) or rabbit β-Tubulin (Sigma Aldrich T8578, 1:1000), and secondary antibodies were goat anti-chicken Alexa Fluor 647 IgY (H+L) (Invitrogen, A-21449, 1:800) or goat anti-rabbit CoraLite488-conjugated IgG (H+L) (Proteintech, SA00013-2, 1:200).

### Statistical Analyses

For biodistribution studies, tissue uptake was calculated in percent injected dose per gram (% ID/g), and groupwise comparisons within each timepoint were performed using Kruskal-Wallis test. For small animal PET-MR data, the ratio of operative leg uptake in kBq/cc divided by non-operative leg uptake was compared between groups and across timepoints using repeated-measures ANOVA, in which group (i.e., unilateral sciatic nerve transection with repair versus transection without repair) was the between-subjects factor and timepoint (i.e., four weeks versus 16 weeks post-injury) was the within-subjects factor. All statistical analyses were performed using SAS software version 9.4 (SAS Institute), and p-values <0.05 were considered statistically significant.

### Example 4

### Clinical Applications of [¹⁸F]DCFPyL PET/CT for Spinal Cord Injury and Brachial Plexopathies

Example 4 demonstrated the utility of [¹⁸F]DCFPyL PET/CT for identifying muscle denervation after peripheral nerve injury. To evaluate the specificity of [¹⁸F]DCFPyL for denervation versus disuse atrophy and its applicability to more proximal peripheral nervous system (PNS) insults, we next imaged three patients with various spinal cord injuries (SCI). Although commonly considered a pathology affecting upper motor neurons (UMNs) of the central nervous system, SCI generally also results in injury to lower motor neurons (LMNs) of the PNS because the cell bodies of LMNs reside within the spinal cord. Consequently, muscles that have lost volitional control from UMN injuries may or may not also be denervated from LMN injuries.

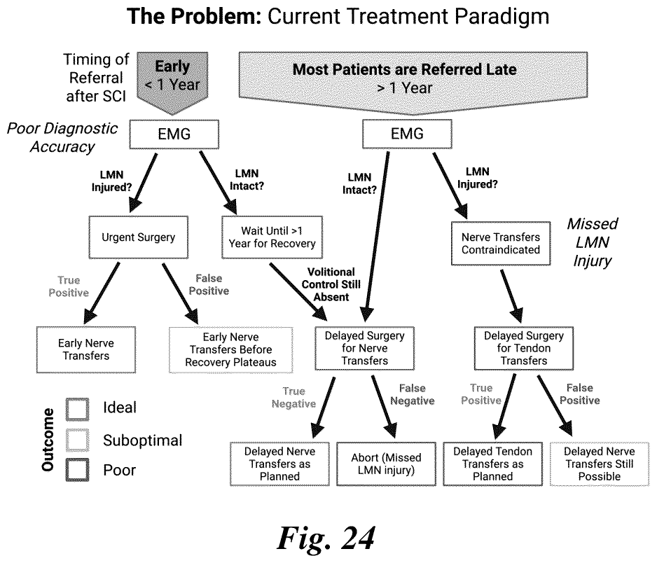

Historically, LMN injuries in SCI were of little relevance because muscles are paralyzed in these patients irrespective of LMN integrity. New therapeutic and surgical strategies for SCI, however, such as peripheral nerve transfers, have made distinguishing innervated from denervated muscles critically important. Nerve transfers involve rerouting peripheral nerves from expendable muscles with volitional control to functionally-important muscles that are paralyzed, thereby transferring volitional control to critical muscles. The timing and success of nerve transfers depends foremost on the integrity of LMNs. If LMNs are injured, nerve transfers must be performed within one year to rescue denervated muscles from irreversible atrophy. If LMNs are intact, nerve transfers can safely be delayed for several years to allow time for potential spontaneous recovery through upper motor neuron (UMN) reorganization. Unfortunately, non-invasive tests for LMN injuries, such as electromyography (EMG), are not reliable in patients with cervical SCI. As a result, LMN injuries are often missed, nerve transfers are either aborted intraoperatively or are performed unnecessarily early, and patient outcomes are often poor (FIG. 24).

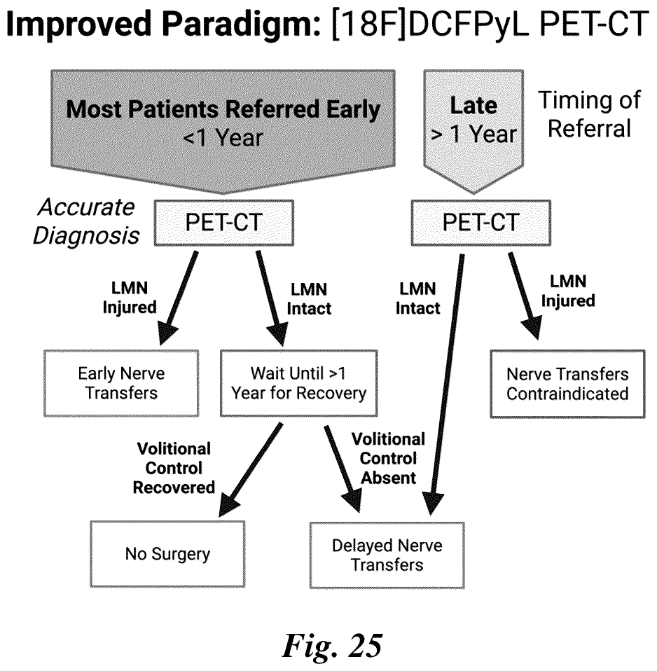

Needle EMG is considered the gold-standard non-invasive diagnostic test for muscle denervation after peripheral nerve injury. EMG, however, is not reliable in patients with SCI, demonstrating only 40% sensitivity and 80% specificity for predicting the results of intraoperative electrical stimulation. Explanations for this uniquely poor performance in SCI care are that (1) patients cannot voluntarily contract muscles due to UMN injuries; (2) sampling a few areas within each muscle misrepresents the overall status of partially denervated muscles; and (3) atrophy, contractures, and involuntary movements complicate both electrode positioning and recordings. Various imaging modalities have been investigated to address these limitations, but none have been able to supplant EMG. Without wishing to be bound to any one particular theory, it is thought that [¹⁸F]DCFPyL PET/CT offers a precise and pragmatic way to evaluate all extremity muscles through a single scan. This approach has promise to improve utilization of peripheral nerve transfers that salvage and restore upper extremity function (FIG. 25).

### Clinical Case 1:

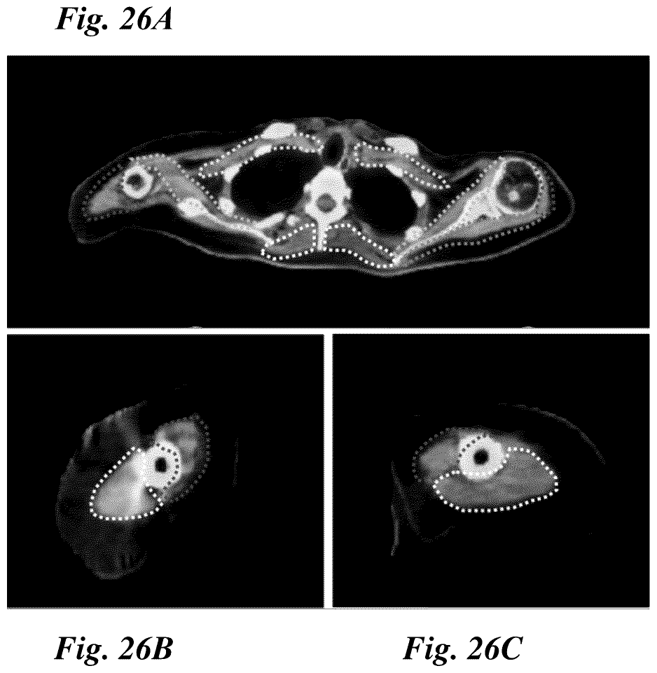

FIG. 26 demonstrates [¹⁸F]DCFPyL PET/CT findings from a 21-year-old man who sustained a complete C4 SCI 16 months prior. This injury produced significant spinal cord damage below C4, and the patient was expected to have LMN damage in at least C5-C6 spinal levels. On exam, the patient had no significant volitional control of his C5-C6 innervated shoulder girdle muscles, elbow flexor muscles (e.g., biceps and brachialis muscles), or triceps muscles. On [¹⁸F]DCFPyL PET/CT, he demonstrated elevated PET uptake in all C5-C6 innervated shoulder girdle muscles and low uptake in his trapezius muscles, which are innervated by the spinal accessory nerve (i.e., CN XI) and were not involved in his injury (FIG. 26A). Moreover, he demonstrated high PET uptake in his biceps/brachialis muscles bilaterally, which are innervated by C5-C7 (FIG. 26B-FIG. 26C). This corroborated the results of needle EMG, which demonstrated denervation of bilateral biceps muscles. Of note, he also had high PET uptake in his right triceps muscle, but low uptake in his left triceps muscle (FIG. 26B-FIG. 26C).

This observation suggested that the left triceps muscle had preserved LMN innervation, and that he would potentially be a candidate for delayed nerve transfers to the left triceps muscle only. This patient went on to receive surgery to restore elbow flexion on his right side. Intraoperative electrical stimulation confirmed that his native right elbow flexor muscles were terminally atrophic and that his trapezius muscle was intact. Accordingly, he received a free functional muscle transfer to restore elbow flexion, in which the right CN XI was utilized to provide innervation to the transferred muscle.

### Clinical Case 2:

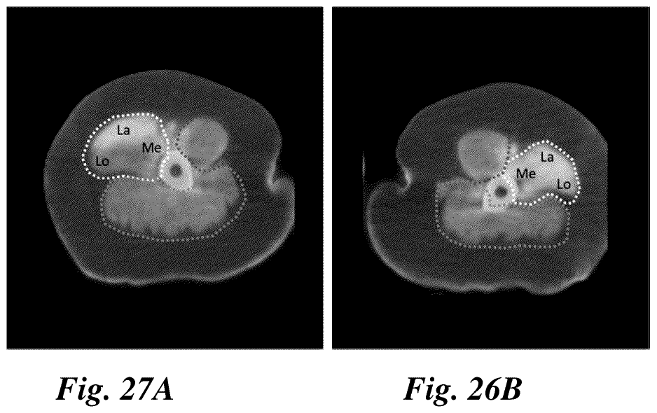

FIG. 27 demonstrates the results of [¹⁸F]DCFPyL PET/CT in a 23-year-old man who sustained an incomplete C6 SCI 20 months prior. On clinical exam, he had normal deltoid function and elbow flexion bilaterally. He also had clinically normal triceps function on the right side, but absent triceps function on the left side. Needle EMG demonstrated denervation in his left triceps muscle, which was corroborated by high [¹⁸F]DCFPyL PET uptake in the left triceps (FIG. 27B). Again, this observation indicated that his left triceps was chronically denervated and would not respond to nerve transfer at this late timepoint. [¹⁸F]DCFPyL PET/CT also revealed high uptake in the right lateral triceps head, which is primarily innervated by C6, suggesting partial muscle denervation that was not captured by clinical exam or EMG (FIG. 27A).

### Clinical Case 3:

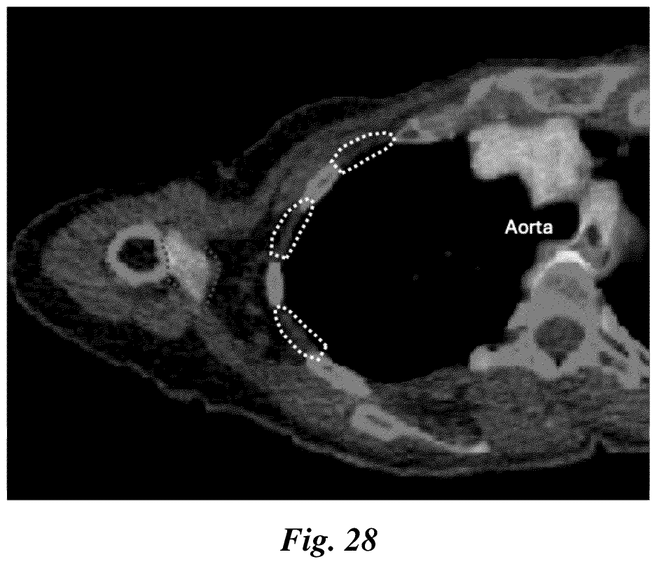

FIG. 28 demonstrates the results of [¹⁸F]DCFPyL PET/CT in a 59-year-old woman who had sustained an incomplete SCI from a spinal arteriovenous malformation (AVM) that spontaneously bled. The zone of injury was predominantly between C4-C6 spinal levels. The patient presented 7 months after this injury with persistent absence of right elbow flexion and was interested in surgical options to restore elbow function. At this relatively early timepoint after injury, the patient would be a candidate for intercostal to musculocutaneous nerve transfer to restore biceps/brachialis muscle function, provided that her intercostal muscles also were not denervated by her injury. Needle EMG of these intercostal muscles, however, was deemed to have an unacceptably high risk of causing pneumothorax by our neurology group, and so it could not be performed. [¹⁸F]DCFPyL PET/CT demonstrated high uptake in her elbow flexors and low uptake in her intercostal muscles, suggesting that she would be a candidate for nerve transfer. This observation highlights an advantage of non-invasive PET/CT over needle EMG for surgical planning.

### Clinical Case 4:

Parsonage-Turner syndrome, also termed neuralgic amyotrophy or brachial plexus neuritis, is a rare yet highly debilitating degenerative neuropathy with an estimated 20-30 new cases per 100,000 individuals annually. Patients typically develop sudden, severe upper extremity pain that is followed by multifocal paralysis and patchy sensory loss. Although spontaneous resolution is possible, most patients develop chronic functional deficits. Prompt nerve surgical intervention can enhance long-term recovery. Diagnosis, however, is typically both delayed and uncertain as there is currently no objective way to identify this condition. While nerve conduction studies (NCS) are considered the diagnostic gold-standard, more than 80% of affected patients actually have normal sensory NCS. On histopathology, Parsonage-Turner syndrome is defined by hourglass-like constrictions (HGCs) along involved nerves, which consist of focal areas of narrowing, fibrosis, axonal injury, and dense inflammation. Whereas high resolution ultrasound (HRUS) and magnetic resonance neurography (MRN) have demonstrated promise for detecting HGCs in patients with electrodiagnostically-confirmed Parsonage-Turner syndrome, their accuracy in the majority of patients without classic electrodiagnostic findings is unclear. Performance of these studies also requires highly specialized technicians and radiologists, which limits their availability. Therefore, the rule for patients with Parsonage-Turner syndrome is a long and frustrating search for answers, with most diagnoses occurring 10 months after symptom onset and more than 60% of patients being initially misdiagnosed with either shoulder pathology or cervical radiculopathy. We investigated whether [¹⁸F]DCFPyL could identify muscle denervation in these patients to assist in localizing HGCs for surgical decompression.

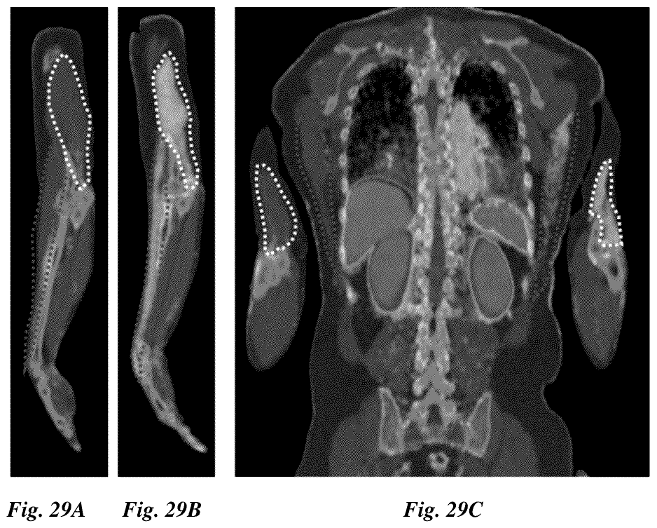

FIG. 29 demonstrates the results of [¹⁸F]DCFPyL PET/CT in a 56-year-old man with a 7-month history of paralysis in his left triceps muscle and wrist/finger extensor muscles that was attributed to Parsonage-Turner syndrome. The patient's symptoms appeared to be localized to the radial nerve. PET/CT, however, suggested that the left latissimus muscle also was also denervated, which had not been identified clinically due to the redundant function of the latissimus muscles with other shoulder girdle muscles. This observation indicated that the lesion was more proximal than the radial nerve, likely localizing instead to the posterior cord of the brachial plexus. The patient subsequently underwent surgical exploration, which confirmed that an HGC was present within the posterior cord.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Aydin M A, Comlekci S, Ozguner M, Cesur G, Nasir S, Aydin Z D. The finfluence of continuous exposure to 50 Hz electric field on nerve regeneration in a rat peroneal nerve crush injury model. Bioelectromagnetics: Journal of the Bioelectromagnetics Society, The Society for Physical Regulation in Biology and Medicine, The European Bioelectromagnetics Association. 2006; 27(5):401-403.
Adhihetty P J et al., Effect of denervation on mitochondrially mediated apoptosis in skeletal muscle. Journal of applied physiology 102, 1143-1151 (2007).
Argadine H M et al., The effect of denervation on protein synthesis and degradation in adult rat diaphragm muscle. Journal of applied physiology 107, 438-444 (2009).
Bailey R, Kaskutas V, Fox I, Baum C M, Mackinnon S E. Effect of upper extremity nerve damage on activity participation, pain, depression, and quality of life. The Journal of hand surgery. 2009; 34(9):1682-1688.
Barinka C, Rojas C, Slusher B, Pomper M. Glutamate carboxypeptidase II in diagnosis and treatment of neurologic disorders and prostate cancer. Current medicinal chemistry. 2012; 19(6):856-870.
Barinka, C. et al., Substrate specificity, inhibition and enzymological analysis of recombinant human glutamate carboxypeptidase II. Journal of neurochemistry 80, 477-487 (2002).
Berger M J, Adewuyi A A, Doherty C, et al. Segmental infralesional lower motor neuron abnormalities in patients with sub-acute traumatic spinal cord injury. medRxiv. 2023:2023.02. 18.23286121.
Berger M J, Robinson L, Krauss E M. Lower motor neuron abnormality in chronic cervical spinal cord injury: implications for nerve transfer surgery. Journal of neurotrauma. 2022; 39(3-4):259-265.
Bertuzzi M, Chang W, Ampatzis K. Adult spinal motoneurons change their neurotransmitter phenotype to control locomotion. Proceedings of the National Academy of Sciences. 2018; 115(42):E9926-E9933.
Birch R, Misra P, Stewart M, et al. Nerve injuries sustained during warfare: part I—epidemiology. The Journal of Bone and Joint Surgery British volume. 2012(a); 94(4):523-528.
Birch R, Misra P, Stewart M, et al. Nerve injuries sustained during warfare: part II: Outcomes. The Journal of Bone and Joint Surgery British volume. 2012(b); 94(4):529-535.
Bois F et al., [68Ga]Ga-PSMA-11 in prostate cancer: a comprehensive review. American Journal of Nuclear Medicine and Molecular Imaging 10, 349 (2020).
Borisov A B, Carlson B M. Cell death in denervated skeletal muscle is distinct from classical apoptosis. The Anatomical Record: An Official Publication of the American Association of Anatomists. 2000; 258(3):305-318.
Brasse-Lagnel C et al., Control of mammalian gene expression by amino acids, especially glutamine. The FEBS journal 276, 1826-1844 (2009).
Bruyns C N, Jaquet J B, Schreuders T A, Kalmijn S, Kuypers P D, Hovius S E. Predictors for return to work in patients with median and ulnar nerve injuries. J Hand Surg Am. Jan 2003; 28(1):28-34.
Cao Y et al., Glutamate carboxypeptidase II gene knockout attenuates oxidative stress and cortical apoptosis after traumatic brain injury. BMC neuroscience 17, 15 (2016).
Carmeli E, Moas M, Reznick A Z, Coleman R. Matrix metalloproteinases and skeletal muscle: a brief review. Muscle & Nerve: Official Journal of the American Association of Electrodiagnostic Medicine. 2004; 29(2):191-197.
Cartwright M S, Chloros G D, Walker F O, Wiesler E R, Campbell W W. Diagnostic ultrasound for nerve transection. Muscle & Nerve: Official Journal of the American Association of Electrodiagnostic Medicine. 2007; 35(6):796-799.
Castanov V, Berger M, Ritsma B, Trier J, Hendry J M. Optimizing the timing of peripheral nerve transfers for functional re-animation in cervical spinal cord injury: a conceptual framework. Journal of Neurotrauma. 2021; 38(24):3365-3375.
Chang S S, Reuter V E, Heston W, Bander N H, Grauer L S, Gaudin P B. Five different anti-prostate-specific membrane antigen (PSMA) antibodies confirm PSMA expression in tumor-associated neovasculature. Cancer research. 1999; 59(13):3192-3198.
Chen Y et al., 2-(3-{1-Carboxy-5-[(6-[18F]Fluoro-Pyridine-3-Carbonyl)-Amino]-Pentyl}-Ureido)-Pentanedioic Acid, [18F]DCFPyL, a PSMA-Based PET Imaging Agent for Prostate Cancer [18F]DCFPyL Synthesis and In Vivo Evaluation. Clinical Cancer Research 17, 7645-7653 (2011).
Chen Y, Dhara S, Banerjee S R, et al. A low molecular weight PSMA-based fluorescent imaging agent for cancer. Biochemical and biophysical research communications. 2009; 390(3):624-629.
Choi J S, Seo H G, Oh B M, et al. 18F-FDG uptake in denervated muscles of patients with peripheral nerve injury. Annals of Clinical and Translational Neurology. 2019; 6(11):2175-2185.
Chouteau W L et al., Interrater reliability of needle electromyographic findings in lumbar radiculopathy. American journal of physical medicine & rehabilitation 89, 561-569 (2010).
Colbert S H, Mackinnon S E. Nerve transfers for brachial plexus reconstruction. Hand clinics. 2008; 24(4):341-361.
Colombo M N, Francolini M. Glutamate at the vertebrate neuromuscular junction: from modulation to neurotransmission. Cells. 2019; 8(9):996.
Conway R E, Petrovic N, Li Z, Heston W, Wu D, Shapiro L H. Prostate-specific membrane antigen regulates angiogenesis by modulating integrin signal transduction. Molecular and cellular biology. 2006; 26(14):5310-5324.
Coyle, JT and Puttfarcken P, Oxidative stress, glutamate, and neurodegenerative disorders. Science 262, 689-695 (1993).
Cushman, D M et al., Complications associated with electromyography: a systematic review. American Journal of Physical Medicine & Rehabilitation 99, 149-155 (2020).
Date A A, Rais R, Babu T, et al. Local enema treatment to inhibit FOLH1/GCPII as a novel therapy for inflammatory bowel disease. Journal of Controlled Release. 2017; 263:132-138.
Daube J R and Rubin D I, Needle electromyography. Muscle & Nerve: Official Journal of the American Association of Electrodiagnostic Medicine 39, 244-270 (2009).
Dinh P, Hazel A, Palispis W, Suryadevara S, Gupta R. Functional assessment after sciatic nerve injury in a rat model. Microsurgery: Official Journal of the International Microsurgical Society and the European Federation of Societies for Microsurgery. 2009; 29(8):644-649.
Ditunno J F, Stover S L, Freed M M, Ahn J. Motor recovery of the upper extremities in traumatic quadriplegia: a multicenter study. Archives of physical medicine and rehabilitation. 1992; 73(5):431-436.
Ehmsen J T, Hoke A. Cellular and molecular features of neurogenic skeletal muscle atrophy. Experimental Neurology. 2020; 331:113379.
English A W, Chen Y, Carp J S, Wolpaw J R, Chen X Y. Recovery of electromyographic activity after transection and surgical repair of the rat sciatic nerve. Journal of neurophysiology. 2007; 97(2):1127-1134.
Evans J C, Malhotra M, Cryan J F, O'Driscoll C M. The therapeutic and diagnostic potential of the prostate specific membrane antigen/glutamate carboxypeptidase II (PSMA/GCPII) in cancer and neurological disease. British journal of pharmacology. 2016; 173(21):3041-3079.
Farinas A F et al., Diffusion magnetic resonance imaging predicts peripheral nerve recovery in a rat sciatic nerve injury model. Plastic and reconstructive surgery 145, 949-956 (2020).
Favero M et al., Synapse formation and elimination: role of activity studied in different models of adult muscle reinnervation. Journal of neuroscience research 85, 2610-2619 (2007).
Ferreira G, Iravani A, Hofman M S, Hicks R J. Intra-individual comparison of 68 Ga-PSMA-11 and 18 F-DCFPyL normal-organ biodistribution. Cancer Imaging. 2019; 19:1-10.
Fontaine C, Yeager E A, Sledziona M, Jones A K, Cheetham J. Revitalizing the common peroneal function index for assessing functional recovery following nerve injury. Brain and Behavior. 2021; 11(2):e01968.
Foss C A, Mease R C, Fan H, et al. Radiolabeled small-molecule ligands for prostate-specific membrane antigen: in vivo imaging in experimental models of prostate cancer. Clinical cancer research. 2005; 11(11):4022-4028.
Fox I K, Davidge K M, Novak C B, et al. Nerve transfers to restore upper extremity function in cervical spinal cord injury: update and preliminary outcomes. Plastic and reconstructive surgery. 2015; 136(4):780-792.
Fox I K, Novak C B, Krauss E M, et al. The use of nerve transfers to restore upper extremity function in cervical spinal cord injury. PM&R. 2018; 10(11):1173-1184. e2.
Fuglsang-Frederiksen A and Pugdahl K, Current status on electrodiagnostic standards and guidelines in neuromuscular disorders. clinical neurophysiology 122, 440-455 (2011).
Giuffre J L, Kakar S, Bishop A T, Spinner R J, Shin A Y. Current concepts of the treatment of adult brachial plexus injuries. The Journal of hand surgery. 2010; 35(4):678-688.
Goodmurphy C et al., The accuracy of needle placement in extremity muscles: a blinded study. Journal of Clinical Neurophysiology 24, 366-378 (2007).
Gordon T et al., Experimental strategies to promote functional recovery after peripheral nerve injuries. Journal of the peripheral nervous system 8, 236-250 (2003).
Gordon T, Tyreman N, Raji M A. The basis for diminished functional recovery after delayed peripheral nerve repair. Journal of Neuroscience. 2011; 31(14):5325-5334.
Hanwright P J, Qiu C, Rath J, et al. Sustained IGF-1 delivery ameliorates effects of chronic denervation and improves functional recovery after peripheral nerve injury and repair. Biomaterials. 2022; 280:121244.
Hargreaves B, Worters P W, Pauly K B, Pauly J M, Koch K M, Gold G E. Metal induced artifacts in MRI. AJR American journal of roentgenology. 2011; 197(3):547.
Hill E J, Fox I K. Current best peripheral nerve transfers for spinal cord injury. Plastic and reconstructive surgery. 2019; 143(1):184e-198e.
Hiltunen J, Suortti T, Arvela S, Seppä M, Joensuu R, Hari R. Diffusion tensor imaging and tractography of distal peripheral nerves at 3 T. Clinical neurophysiology. 2005; 116(10):2315-2323.
Höke A. Mechanisms of Disease: what factors limit the success of peripheral nerve regeneration in humans?Nature clinical practice Neurology. 2006; 2(8):448-454.
Horwath O et al., Ageing leads to selective type II myofibre deterioration and denervation independent of reinnervative capacity in human skeletal muscle. Experimental Physiology 110, 277-292 (2025).
Houdek, MT and Shin A J, Management and complications of traumatic peripheral nerve injuries. Hand Clinics 31, 151-163 (2015).
Huang T C, Blanks R H, Berns M W, Crumley R L. Laser vs. suture nerve anastomosis. Otolaryngology-head and neck surgery. 1992; 107(1):14-20.
Hundepool C A, Bulstra L F, Kotsougiani D, et al. Comparable functional motor outcomes after repair of peripheral nerve injury with an elastase-processed allograft in a rat sciatic nerve model. Microsurgery. 2018; 38(7):772-779.
Ijkema-Paassen J., et al., Reinnervation of muscles after transection of the sciatic nerve in adult rats. Muscle & nerve 25, 891-897 (2002).
Ismail M S, Peters D E, Rowe S P, et al. PSMA-Targeted PET Radiotracer [18F]DCFPyL as an Imaging Biomarker in Inflammatory Bowel Disease. Clinical and Experimental Gastroenterology. 2023:237-247.
Jain N S et al., Evaluation for late nerve transfer surgery in spinal cord injury: predicting the degree of lower motor neuron injury. The Journal of hand surgery 45, 95-103 (2020).
Jan, M M et al., EMG related anxiety and pain: a prospective study. Canadian journal of neurological sciences 26, 294-297 (1999).
Jansen B H, Kramer G M, Cysouw M C, et al. Healthy tissue uptake of 68Ga-prostate-specific membrane antigen, 18F-DCFPyL, 18F-fluoromethylcholine, and 18F-dihydrotestosterone. Journal of Nuclear Medicine. 2019; 60(8):1111-1117.
Javeed S, Dibble C F, Greenberg J K, et al. Upper limb nerve transfer surgery in patients with tetraplegia. JAMA Network Open. 2022; 5(11):e2243890-e2243890.
Jeon T, Fung M M, Koch K M, Tan E T, Sneag D B. Peripheral nerve diffusion tensor imaging: overview, pitfalls, and future directions. Journal of Magnetic Resonance Imaging. 2018; 47(5):1171-1189.
Jewell J L et al., Differential regulation of mTORC1 by leucine and glutamine. Science 347, 194-198 (2015).
Jones D, Pierpaoli C. Contribution of cardiac pulsation to variability of tractography results. 2005:222.
Khalifeh J M, Dibble C F, Van Voorhis A, et al. Nerve transfers in the upper extremity following cervical spinal cord injury. Part 1: systematic review of the literature. Journal of Neurosurgery: Spine. 2019; 31(5):629-640.
Kim Y C and Guan K-L, mTOR: a pharmacologic target for autophagy regulation. The Journal of clinical investigation 125, 25-32 (2015).
Lee S H, Oh B-M, Lee G, Choi H, Cheon G J, Lee S—U. Feasibility of 18F-FDG PET as a noninvasive diagnostic tool of muscle denervation: a preliminary study. Journal of Nuclear Medicine. 2014; 55(10):1737-1740.
Lee S H, Seo H G, Oh B-M, Choi H, Cheon G J, Lee S—U. 18F-FDG positron emission tomography as a novel diagnostic tool for peripheral nerve injury. Journal of neuroscience methods. 2019; 317:11-19.
Lee S K, Wolfe S W. Peripheral nerve injury and repair. JAAOS-Journal of the American Academy of Orthopaedic Surgeons. 2000; 8(4):243-252.
Lisle D, Johnstone S. Usefulness of muscle denervation as an MRI sign of peripheral nerve pathology. Australasian radiology. 2007; 51(6):516-526.
London Z N et al., Altering electromyography studies: importance of the electromyographer's perception of patient pain. Archives of Physical Medicine and Rehabilitation 95, 39-42 (2014).
London Z N, Safety and pain in electrodiagnostic studies. Muscle & nerve 55, 149-159 (2017).
MacDonald E M, Andres-Mateos E, Mejias R, et al. Denervation atrophy is independent from Akt and mTOR activation and is not rescued by myostatin inhibition. Disease models & mechanisms. 2014; 7(4):471-481.
Mackin G A et al., Restoring hand function in patients with severe polyneuropathy: the role of electromyography before tendon transfer surgery. The Journal of hand surgery 24, 732-742 (1999).
Mange K C, Marino R J, Gregory P C, Herbison G J, Ditunno J F. Course of motor recovery in the zone of partial preservation in spinal cord injury. Archives of physical medicine and rehabilitation. 1992; 73(5):437-441.
Marmiroli et al., Tissue distribution of glutamate carboxypeptidase II (GCPII) with a focus on the central and peripheral nervous system. Current medicinal chemistry 19, 1277-1281 (2012).
Menovsky T, Beek J F. Laser, fibrin glue, or suture repair of peripheral nerves: a comparative functional, histological and morphometric study in the rat sciatic nerve. Journal of neurosurgery. 2001; 95(4):694-699.
Midha R, Grochmal J. Surgery for nerve injury: current and future perspectives: JNSPG 75th Anniversary Invited Review Article. Journal of neurosurgery. 2019; 130(3):675-685.
Mulcahey M et al., Evaluation of the lower motor neuron integrity of upper extremity muscles in high level spinal cord injury. Spinal Cord 37, 585-591 (1999).
Mulcahey M, Smith B, Betz R. Evaluation of the lower motor neuron integrity of upper extremity muscles in high level spinal cord injury. Spinal Cord. 1999; 37(8):585-591.
Muller F L et al., Denervation-induced skeletal muscle atrophy is associated with increased mitochondrial ROS production. American journal of physiology-Regulatory, integrative and comparative physiology 293, R1159-Ri168 (2007).
Nam J W, Lee M J, Kim H J. Diagnostic Efficacy of 18 F-FDG PET/MRI in Peripheral Nerve Injury Models. Neurochemical research. 2019; 44:2092-2102.
Nguyen T et al., Uncovering the role of N-acetyl-aspartyl-glutamate as a glutamate reservoir in cancer. Cell reports 27, 491-501. e496 (2019).
Novak, C B, et al., Biomedical and psychosocial factors associated with disability after peripheral nerve injury. JBJS 93, 929-936 (2011).
O'Keefe D S, Bacich D J, Huang S S, Heston W D. A perspective on the evolving story of PSMA biology, PSMA-based imaging, and endoradiotherapeutic strategies. Journal of Nuclear Medicine. 2018; 59(7):1007-1013.
O'Leary, MF, Denervation-induced mitochondrial dysfunction and autophagy in skeletal muscle of apoptosis-deficient animals. American Journal of Physiology-Cell Physiology 303, C447-C454 (2012).
O'shea K, Feinberg J, Wolfe S. Imaging and electrodiagnostic work-up of acute adult brachial plexus injuries. Journal of Hand Surgery (European Volume). 2011; 36(9):747-759.
Padovano W M, Dengler J, Patterson M M, et al. Incidence of nerve injury after extremity trauma in the United States. Hand. 2022; 17(4):615-623.
Pak K, Shin M J, Hwang S-J, et al. Longitudinal changes in glucose metabolism of denervated muscle after complete peripheral nerve injury. Molecular imaging and biology. 2016; 18:741-747.
Pannell W C, Heckmann N, Alluri R K, Sivasundaram L, Stevanovic M, Ghiassi A. Predictors of nerve injury after gunshot wounds to the upper extremity. Hand. 2017; 12(5):501-506.
Panov A et al., The neuromediator glutamate, through specific substrate interactions, enhances mitochondrial ATP production and reactive oxygen species generation in nonsynaptic brain mitochondria. Journal of Biological Chemistry 284, 14448-14456 (2009).
Parida G K, Roy S G, Kumar R. FDG-PET/CT in skeletal muscle: pitfalls and pathologies. Elsevier; 2017:362-372.
Penna V, Wewetzer K, Munder B, Stark G B, Lang E M. The long-term functional recovery of repair of sciatic nerve transection with biogenic conduits. Microsurgery. 2012; 32(5):377-382.
Personius K E et al., Neuromuscular NMDA receptors modulate developmental synapse elimination. Journal of Neuroscience 36, 8783-8789 (2016).
Personius K E, Siebert D, Koch D W, Udin S B. Blockage of neuromuscular glutamate receptors impairs reinnervation following nerve crush in adult mice. Front Cell Neurosci. 2022 Sep. 22; 16:1000218.
Pienta K J et al., A phase 2/3 prospective multicenter study of the diagnostic accuracy of prostate specific membrane antigen PET/CT with 18F-DCFPyL in prostate cancer patients (OSPREY). The Journal of urology 206, 52-61 (2021).
Preston, DC and Shapiro B E, Needle electromyography: Fundamentals, normal and abnormal patterns. Neurologic clinics 20, 361-396 (2002).
Pridmore M D, Glassman G E, Pollins A C, et al. Initial findings in traumatic peripheral nerve injury and repair with diffusion tensor imaging. Annals of Clinical and Translational Neurology. 2021; 8(2):332-347.
Quy P N et al., Proteasome-dependent activation of mammalian target of rapamycin complex 1 (mTORC1) is essential for autophagy suppression and muscle remodeling following denervation. Journal of Biological Chemistry 288, 1125-1134 (2013).
Rahn K A et al., Glutamate in CNS neurodegeneration and cognition and its regulation by GCPII inhibition. Current medicinal chemistry 19, 1335-1345 (2012).
Rennie M J et al., Skeletal muscle glutamine transport, intramuscular glutamine concentration, and muscle-protein turnover. Metabolism 38, 47-51 (1989).
Rivera J C, Glebus G P, Cho M S. Disability following combat-sustained nerve injury of the upper limb. Bone Joint J. February 2014; 96—B(2):254-8. doi:10.1302/0301-620X.96B2.31798
Roberts M J et al., Using PSMA imaging for prognostication in localized and advanced prostate cancer. Nature Reviews Urology 20, 23-47 (2023).
Robinson L R. How electrodiagnosis predicts clinical outcome of focal peripheral nerve lesions. Muscle & nerve. 2015; 52(3):321-333.
Rovenská M, Hlouchová K, Šácha P, et al. Tissue expression and enzymologic characterization of human prostate specific membrane antigen and its rat and pig orthologs. The Prostate. 2008; 68(2):171-182.
Sakuma M, Gorski G, Sheu S H, et al. Lack of motor recovery after prolonged denervation of the neuromuscular junction is not due to regenerative failure. European Journal of Neuroscience. 2016; 43(3):451-462.
Saltzman E B, Villa J C, Doty S B, Feinberg J H, Lee S K, Wolfe S W. A comparison between two collagen nerve conduits and nerve autograft: a rat model of motor nerve regeneration. The Journal of hand surgery. 2019; 44(8):700. e1-700. e9.
Sarhane K A, Slavin B R, Hricz N, et al. Defining the relative impact of muscle versus Schwann cell denervation on functional recovery after delayed nerve repair. Experimental Neurology. 2021; 339:113650.
Scholz T, Pharaon M, Evans G R. Peripheral nerve anatomy for regeneration studies in pigs: feasibility of large animal models. Annals of plastic surgery. 2010; 65(1):43-47.
Schreiber J J et al., Preoperative donor nerve electromyography as a predictor of nerve transfer outcomes. The Journal of Hand Surgery 39, 42-49 (2014).
Selivanov V A et al., Unveiling a key role of oxaloacetate-glutamate interaction in regulation of respiration and ROS generation in nonsynaptic brain mitochondria using a kinetic model. PLoS One 16, e0255164 (2021).
Simon N G, Spinner R J, Kline D G, Kliot M. Advances in the neurological and neurosurgical management of peripheral nerve trauma. Journal of Neurology, Neurosurgery & Psychiatry. 2016; 87(2):198-208.
Smith D H, Burrell J C, Browne K D, et al. Tissue-engineered grafts exploit axon-facilitated axon regeneration and pathway protection to enable recovery after 5-cm nerve defects in pigs. Science Advances. 2022; 8(44):eabm3291.
Steensma B R, Luttje M, Voogt I J, et al. Comparing signal-to-noise ratio for prostate imaging at 7T and 3T. Journal of Magnetic Resonance Imaging. 2019; 49(5):1446-1455.
Strommen, JA, and Daube J R, Determinants of pain in needle electromyography. Clinical neurophysiology 112, 1414-1418 (2001).
Su Y et al., GCPII Inhibition in Macrophages Delays Age-related Muscle Atrophy in Mice. Physiology 39, 2379 (2024).
Swett J E, Hong C Z, Miller P G. All peroneal motoneurons of the rat survive crush injury but some fail to reinnervate their original targets. Journal of comparative neurology. 1991; 304(2):234-252.
Szabo, Z., Mena, E., Rowe, S. P., Plyku, D., Nidal, R., Eisenberger, M. A., . . . & Pomper, M. G. (2015). Initial evaluation of [18 F]DCFPyL for prostate-specific membrane antigen (PSMA)-targeted PET imaging of prostate cancer. Molecular imaging and biology, 17, 565-574.
Tallon C, Sharma A, Zhang Z, et al. Dendrimer-2PMPA Delays Muscle Function Loss and Denervation in a Murine Model of Amyotrophic Lateral Sclerosis. Neurotherapeutics. 2022; 19(1):274-288.
Tan E T, Zochowski K C, Sneag D B. Diffusion MRI fiber diameter for muscle denervation assessment. Quantitative Imaging in Medicine and Surgery. 2022; 12(1):80.
Tang P, Whiteman D R, Voigt C, Miller M C, Kim H. No difference in outcomes detected between decellular nerve allograft and cable autograft in rat sciatic nerve defects. JBJS. 2019; 101(10):e42.
Taylor C A, Braza D, Rice J B, Dillingham T. The incidence of peripheral nerve injury in extremity trauma. American journal of physical medicine & rehabilitation. 2008; 87(5):381-385.
Toros T, Karabay N, Ozaksar K, Sugun T, Kayalar M, Bal E. Evaluation of peripheral nerves of the upper limb with ultrasonography: a comparison of ultrasonographic examination and the intra-operative findings. The Journal of Bone and Joint Surgery British volume. 2009; 91(6):762-765.
Turinsky J and Long C L, Free amino acids in muscle: effect of muscle fiber population and denervation. American Journal of Physiology-Endocrinology and Metabolism 258, E485-E491 (1990).
van Zyl N, Hill B, Cooper C, Hahn J, Galea M P. Expanding traditional tendon-based techniques with nerve transfers for the restoration of upper limb function in tetraplegia: a prospective case series. The Lancet. 2019; 394(10198):565-575.
Watakabe T, Toya R, Saito T, et al. High spatial resolution digital positron emission tomography images with dedicated source-to-background algorithm for radiotherapy planning. Anticancer Research. 2020; 40(5):2567-2572.
Waters R L, Adkins R H, Yakura J S, Sie I. Motor and sensory recovery following complete tetraplegia. Archives of physical medicine and rehabilitation. 1993; 74(3):242-247.
Weber M-A, Wolf M, Wattjes M P. Imaging patterns of muscle atrophy. Thieme Medical Publishers; 2018:299-306.
Widjaja E, Mahmoodabadi S, Rea D, Moineddin R, Vidarsson L, Nilsson D. Effects of gradient encoding and number of signal averages on fractional anisotropy and fiber density index in vivo at 1.5 tesla. Acta Radiologica. 2009; 50(1):106-113.
Yamamoto T et al., Antinociceptive effects of N-acetylaspartylglutamate (NAAG) peptidase inhibitors ZJ-11, ZJ-17 and ZJ-43 in the rat formalin test and in the rat neuropathic pain model. European Journal of Neuroscience 20, 483-494 (2004).
Yang X et al., Denervation drives skeletal muscle atrophy and induces mitochondrial dysfunction, mitophagy and apoptosis via miR-142a-5p/MFN1 axis. Theranostics 10, 1415 (2020).
Zhang Z, Bassam B, Thomas A G, et al. Maternal inflammation leads to impaired glutamate homeostasis and up-regulation of glutamate carboxypeptidase II in activated microglia in the fetal/newborn rabbit brain. Neurobiology of disease. 2016; 94:116-128.
Zhou Y, Narayana P A, Kumaravel M, Athar P, Patel V S, Sheikh K A. High resolution diffusion tensor imaging of human nerves in forearm. Journal of Magnetic Resonance Imaging. 2014; 39(6):1374-1383.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A method for diagnosing a peripheral nervous system (PNS) neuropathy, the method comprising administering to a subject in need of treatment thereof, at least one of a translocator protein (TSPO)-targeting compound or a PSMA-targeting compound and taking an image.

2. The method of claim 1, wherein the peripheral nervous system (PNS) neuropathy includes a PNS insult and/or neuropathy, a recovery of a PNS insult and/or neuropathy, and muscle denervation, including muscle denervation-induced muscle atrophy, and/or muscle re-innervation, and wherein the PNS neuropathy is selected from a peripheral nerve injury (PNI), a brachial plexus injury (BPI), and a spinal cord injury (SCI), including a stretch-related PNI, a laceration, a compression PNI, and a PNI related to one or more conditions selected from radiation, electricity, injection, crush, cold, and an intra-neural or extra-neural pathology.

3. The method of claim 1, wherein the TSPO-targeting compound comprises a compound of formula (I): 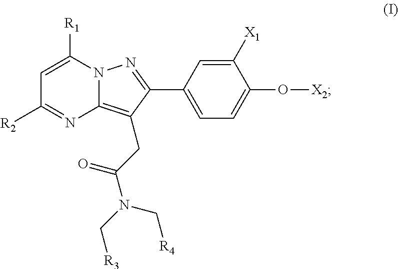
wherein:
X₁ can be present or absent and when present is selected from a radioisotope of fluorine, a radioisotope of iodine, a radioisotope of bromine, and a radioisotope of astatine;
under the proviso that when X₁ is present, X₂ is H or C₁-C₄ alkyl and when X₁ is absent, X₂ is -L-I, wherein L is a linker and I is an imaging agent; and
R₁, R₂, R₃ and R₄ are each independently selected from hydroxyl, C₁ to C₁₀ alkyl, cycloalkyl, aryl, alkylamino, alkylamino, alkenyl, alkynyl, hydroxyalkyl, alkoxyl, dialkylamino thioalkyl, thioalkenyl, thioalkynyl, aryloxy, acyloxy, thioacyl, amido, and sulphonamido; wherein each of alkyl, or aryl moiety may be unsubstituted or substituted with one or more substituents selected from the group consisting of halo, hydroxyl, carboxyl, phosphoryl, phosphonyl, phosphono C₁-C₆ alkyl, carboxy C₁-C₆ alkyl, dicarboxy C₁-C₆ alkyl, dicarboxy halo C₁-C₆ alkyl, sulfonyl, cyano, nitro, alkoxy, alkylthio, acyl, acyloxy, thioacyl, acylthio, aryloxy, amino, alkylamino, dialkylamino, trialkylamino, arylalkylamino, guanidino, aldehydo, ureido, and aminocarbonyl, an amino acid residue, and a substituted amino acid residue; and
pharmaceutically acceptable salts thereof.

4. The method of claim 3, wherein the compound of formula (I) is a compound of formula (Ia): 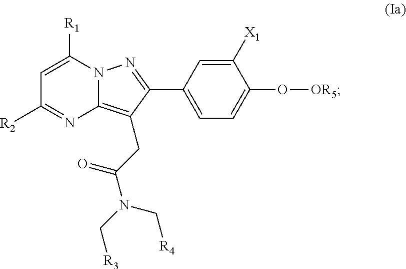
wherein R₅ is H or C₁-C₄ alkyl, wherein X₁ is selected from ¹⁸F, ¹²³I, ¹²⁴I, ¹²⁵I, ¹³¹I, ⁷⁵Br, ⁷⁶Br, ⁷⁷Br, ⁸⁰Br, ^{80m}Br, ⁸²Br, ⁸³Br and ²¹¹At.

5. The method of claim 4, wherein the compound of formula (Ia) is:

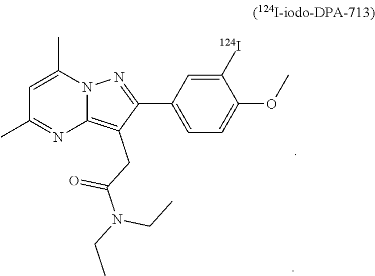

6. The method of claim 3, wherein:
(a) X₁ is ¹²³I or ¹²⁵I and the imaging is single photon emission computed tomography (SPECT); or
(b) X₁ is ¹⁸F or ¹²⁴I and the imaging is positron emission tomography (PET).

7. The method of claim 3, wherein the compound of formula (I) is a compound of formula (Ib):

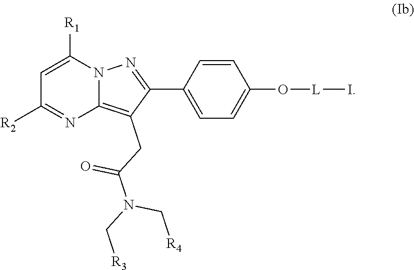

8. The method of claim 7, wherein the compound of formula (Ib) is: 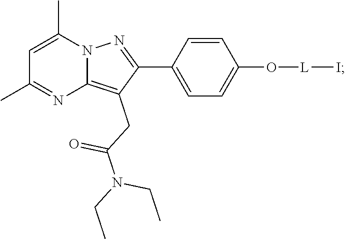
wherein:
L comprises an alkylene linker comprising between 1 to 10 carbon atoms; and
I comprises an imaging agent covalently linked to the linker, L, via an amide linkage.

9. The method of claim 8, wherein the imaging agent comprises an optical dye.

10. The method of claim 11, wherein the optical dye comprises a fluorescent dye.

11. The method of claim 10, wherein the fluorescent dye comprises a fluorescent dye that emits in the near infrared spectral region.

12. The method of claim 10, wherein:
(a) the fluorescent dye is selected from a polymethine dye, a coumarin dye, a xanthene dye, and a boron-dipyrromethene (BODIPY) dye;
(b) the polymethine dye is selected from a carbocyanine dye, an indocarbocyanine dye, an oxacarbocyanine dye, a thiacarbocyanine dye, and a merocyanine dye; and
(c) the xanthene dye is selected from a fluorescein dye and a coumarin dye.

13. The method of claim 10, wherein the fluorescent dye is selected from:
BODIPY FL, BODIPY R6G, BODIPY TR, BODIPY TMR, BODIPY 493/503, BODIPY 530/550, BODIPY 558/568, BODIPY 564/570, BODIPY 576/589, BODIPY 581/591, BODIPY 630/650, and BODIPY 650/665;
VivoTag-645, VivoTag-680, VivoTag-S680, VivoTag-S750, VivoTag-800;
Alexa Fluor 488, Alexa Fluor 532, Alexa Fluor 546, Alexa Fluor 555, Alexa Fluor 568, Alexa Fluor 594, Alexa Fluor 647, Alexa Fluor 660, Alexa Fluor 680, Alexa Fluor 700, Alexa Fluor 750, and AlexaFluor790;
Dy677, Dy676, Dy682, Dy752, Dy780;
DyLight 350, DyLight 405, DyLight 488, DyLight 547, DyLight 550, DyLight 594, DyLight 633, DyLight 647, DyLight 650, DyLight 680, DyLight 755, and DyLight 800;
HiLyte Fluor 405, HiLyte Fluor 488, HiLyte Fluor 532, HiLyte Fluor 555, HiLyte™ Fluor 594, HiLyte Fluor 647, HiLyte Fluor 680, HiLyte Fluor 750;
IR800 (Dimethyl{4-[1,5,5-tris(4-dimethylaminophenyl)-2,4-pentadienylidene]-2,5-cyclohexadien-1-ylidene}ammonium perchlorate);
IRDye 650, IRDye 680RD, IRDye 680LT, IRDye 700, IRDye 700DX, IRDye 750, IRDye 800, IRDye 800CW, IRDye 800RS; and
ADS1065A, ADS1075A, ADS775MI, ADS775MP, ADS775PI, ADS775PP, ADS780HO, ADS780WS, ADS785WS, ADS790WS, ADS795WS, ADS798SM, ADS800AT, ADS815EI, ADS830AT, ADS830WS, ADS832WS, ADS845MC, and ADS920MC.

14. The method of claim 9, wherein the optical dye is selected from:

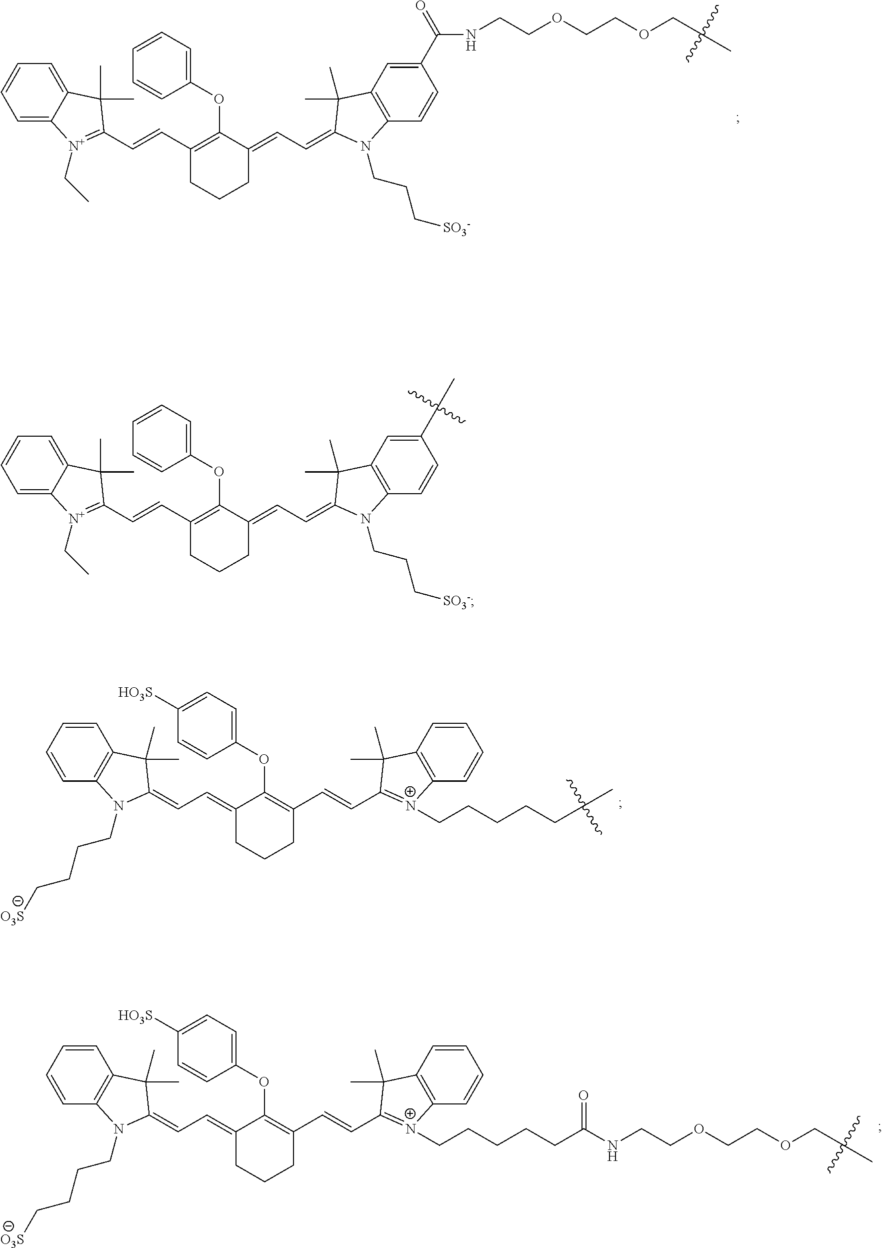

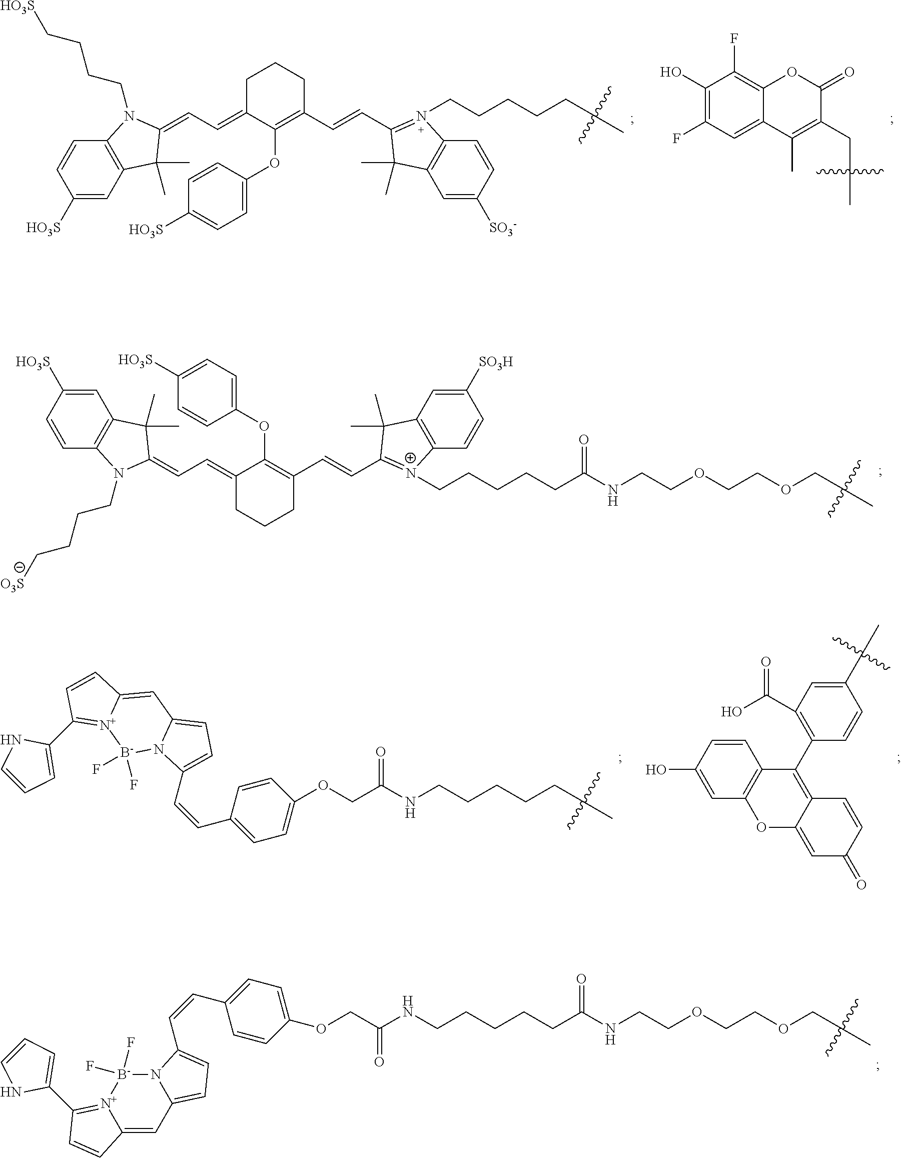

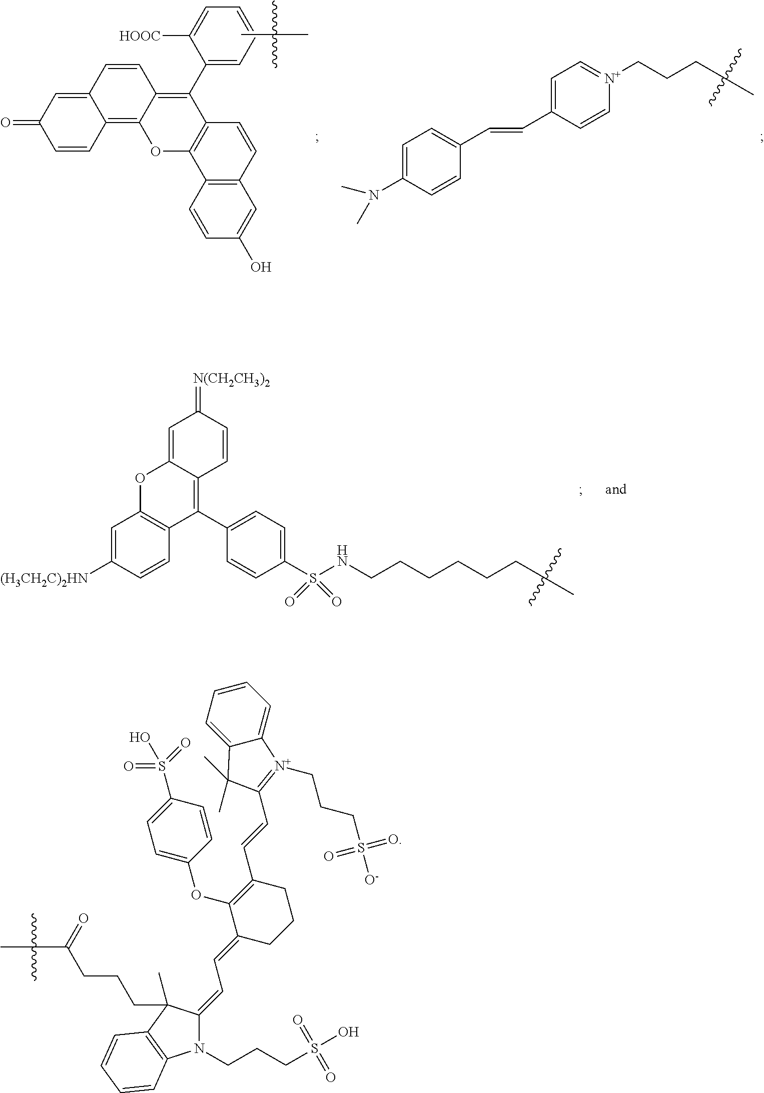

15. The method of claim 1, wherein the PSMA-targeting compound is a compound of formula (II): 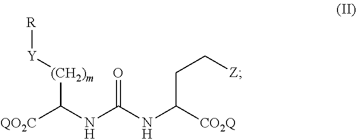
wherein:
Z is tetrazole or CO₂Q;
each Q is independently selected from hydrogen or a protecting group;
and wherein:
(A) m is 0, 1, 2, 3, 4, 5, or 6;
R is a pyridine ring selected from: 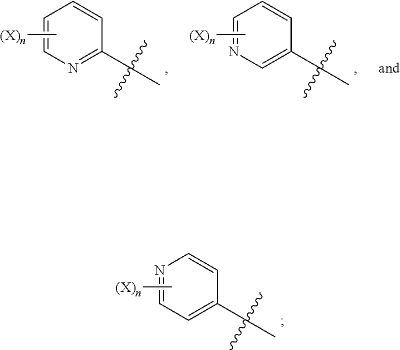
wherein:
X is fluorine, iodine, a radioisotope of fluorine, a radioisotope of iodine, chlorine, bromine, a radioisotope of bromine, a radioisotope of astatine, NO₂, NH₂, N⁺(R²)₃, Sn(R²)₃, Si(R²)₃, Hg(R²), B(OH)₂, —NHNH₂, —NHN═CHR³, —NHNH—CH₂R³;
n is 1, 2, 3, or 4;
Y is O, S, N(R′), C(O), NR′C(O), C(O)N(R′), OC(O), C(O)O, NR′C(O)NR′, NR′C(S)NR′, NR′S(O)₂, S(CH₂)ₚ, NR′(CH₂)ₚ, O(CH₂)ₚ, OC(O)CHR⁸NHC(O), NHC(O)CHR⁸NHC(O), or a covalent bond; wherein p is 1, 2, or 3, R′ is H or C₁-C₆ alkyl, and R′ is hydrogen, alkyl, aryl or heteroaryl, each of which may be substituted;
R² is C₁-C₆ alkyl; and
R³ is alkyl, alkenyl, alkynyl, aryl, or heteroaryl each of which is substituted by fluorine, iodine, a radioisotope of fluorine, a radioisotope of iodine, chlorine, bromine, a radioisotope of bromine, or a radioisotope of astatine, NO₂, NH₂, N⁺(R²)₃, Sn(R²)₃, Si(R²)₃, Hg(R²), or B(OH)₂; or
(B) m is 0, 1, 2, 3, 4, 5, or 6;
Y is O, S, N(R′), C(O), NR′C(O), C(O)N(R′), OC(O), C(O)O, NR′C(O)NR′, NR′C(S)NR′, NR′S(O)₂, S(CH₂)ₚ, NR′(CH₂)ₚ, O(CH₂)ₚ, OC(O)CHR⁸NHC(O), NHC(O)CHR⁸NHC(O), or a covalent bond; wherein p is 1, 2, or 3, R′ is H or C₁-C₆ alkyl, and R′ is hydrogen alkyl, aryl or heteroaryl, each of which may be substituted;
R is: 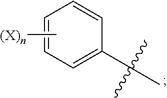
wherein:
X′ is selected from the group consisting of NHNII₂, —NHN═CHR³, and —NHNH—CH₂R³; wherein R³ is alkyl, alkenyl, alkynyl, aryl, or heteroaryl each of which is substituted by fluorine, iodine, a radioisotope of fluorine, a radioisotope of iodine, bromine, a radioisotope of bromine, or a radioisotope of astatine; NO₂, NH₂, N⁺(R²)₃, Sn(R²)₃, Si(R²)₃, Hg(R²), or B(OH)₂;
R² is C₁-C₆ alkyl;
n is 1, 2, 3, 4, or 5; or
(C) m is 4,
Y is NR′, and
R is: 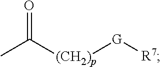
wherein G is O, NR′ or a covalent bond;
R is H or C₁-C₆ alkyl; p is 1, 2, 3, or 4, and
R⁷ is selected from the group consisting of NH₂, N═CHR³, NH—CH₂R³, wherein R³ is alkyl, alkenyl, alkynyl, aryl, or heteroaryl each of which is substituted by fluorine, iodine, a radioisotope of fluorine, a radioisotope of iodine, chlorine, bromine, a radioisotope of bromine, or a radioisotope of astatine, NO₂, NH₂, N⁺(R²)₃, Sn(R²)₃, Si(R²)₃, Hg(R²), B(OH)₂; and R² is C₁-C₆ alkyl.

16. The method of claim 15, wherein the compound of formula (II) has the structure: 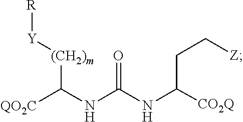
wherein m is 0, 1, 2, 3, 4, 5, or 6;
R is a pyridine ring selected from the group consisting of: 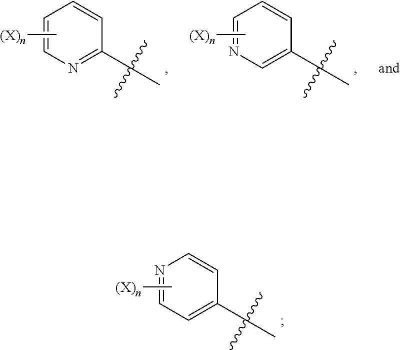
wherein:
X is fluorine, iodine, a radioisotope of fluorine, a radioisotope of iodine, chlorine, bromine, a radioisotope of bromine, a radioisotope of astatine, NO₂, NH₂, N⁺(R²)₃, Sn(R²)₃, Si(R²)₃, Hg(R²), B(OH)₂, —NHNH₂, —NHN═CHR³, and —NHNH—CH₂R³;
each Q is independently selected from hydrogen or a protecting group;
Y is O, S, N(R′), C(O), NR′C(O), C(O)N(R′), OC(O), C(O)O, NR′C(O)NR′, NR′C(S)NR′, NR′S(O)₂, S(CH₂)ₚ, NR′(CH₂)ₚ, O(CH₂)ₚ, OC(O)CHR⁸NHC(O), NHC(O)CHR⁸NHC(O), or a covalent bond; wherein p is 1, 2, or 3, R′ is H or C₁-C₆ alkyl, and R′ is hydrogen, alkyl, aryl or heteroaryl, each of which may be substituted;
Z is tetrazole or CO₂Q;
R² is C₁-C₆ alkyl; and
R³ is alkyl, alkenyl, alkynyl, aryl, or heteroaryl, each of which is substituted by fluorine, iodine, a radioisotope of fluorine, a radioisotope of iodine, chlorine, bromine, a radioisotope of bromine, or a radioisotope of astatine; NO₂, NH₂, N⁺(R²)₃, Sn(R²)₃, Si(R²)₃, Hg(R²), or B(OH)₂.

17. The method of claim 16, wherein the compound of formula (II) has the structure: 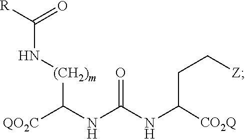
wherein m is not 0.

18. The method of claim 15, wherein the compound of formula (II) has the structure: 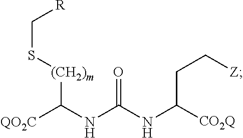
wherein m is not 0.

19. The method of claim 15, wherein for the compound of formula (II):
m is 0, 1, 2, 3, 4, 5, or 6;
Y is O, S, N(R′), C(O), NR′C(O), C(O)N(R′), OC(O), C(O)O, NR′C(O)NR′, NR′C(S)NR′, NR′S(O)₂, S(CH₂)ₚ, NR′(CH₂)ₚ, O(CH₂)ₚ, OC(O)CHR⁸NHC(O), NHC(O)CHR⁸NHC(O), or a covalent bond; wherein p is 1, 2, or 3, R′ is H or C₁-C₆ alkyl, and R⁸ is hydrogen, alkyl, aryl or heteroaryl, each of which may be substituted;
R is 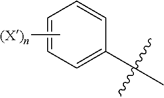
wherein
X′ is selected from the group consisting of NHNH₂, —NHN═CHR³, —NHNH—CH₂R³; wherein R³ is alkyl, alkenyl, alkynyl, aryl, or heteroaryl, each of which is substituted by fluorine, iodine, a radioisotope of fluorine, a radioisotope of iodine, bromine, a radioisotope of bromine, or a radioisotope of astatine; NO₂, NH₂, N⁺(R²)₃, Sn(R²)₃, Si(R²)₃, Hg(R²), and B(OH)₂, where R² is C₁-C₆ alkyl;
n is 1, 2, 3, 4, or 5.

20. The method of claim 15, wherein for the compound of formula (II), X or X′ is fluorine, iodine, or a radioisotope of fluorine or iodine, bromine, a radioisotope of bromine, or a radioisotope of astatine.

21. The method of claim 20, wherein for the compound of formula (II), X or X′ is selected from ¹⁸F, ¹²³I, ¹²⁴I, ¹²⁵I, ¹³¹I, ⁷⁵Br, ⁷⁶Br, ⁷⁷Br, ⁸⁰Br, ^{80m}Br, ⁸²Br, ⁸³Br and ²¹¹At.

22. The method of claim 15, wherein the compound of formula (II) is selected from:

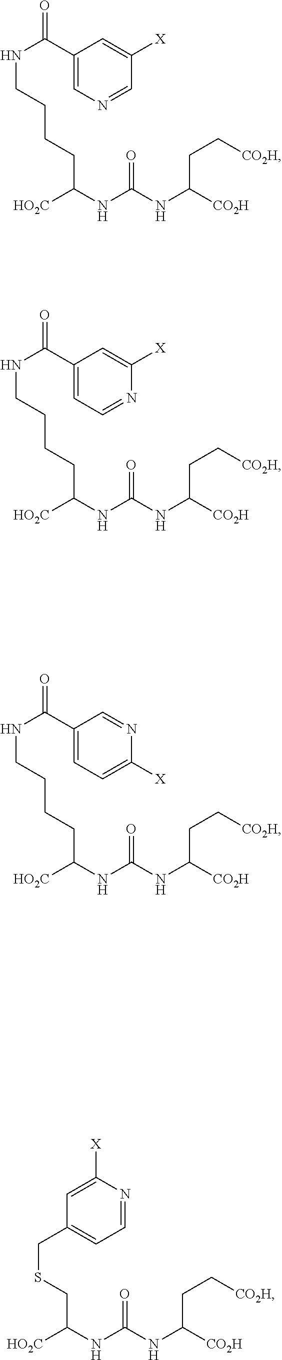

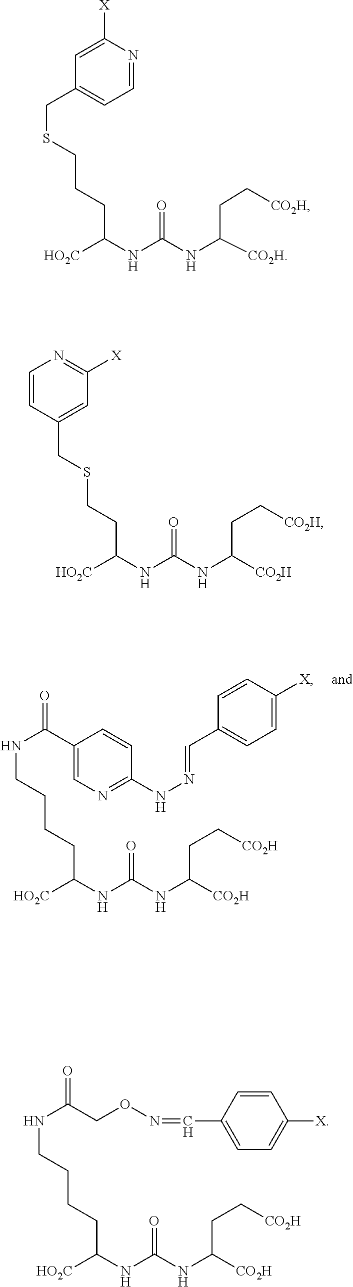

23. The method of claim 15, wherein the compound of formula (II) is:

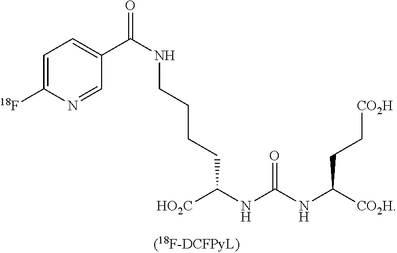 [SMILES: O=C(O)CC[C@H](NC(=O)N[C@@H](CCCCNC(=O)c1ccc([18F])nc1)C(=O)O)C(=O)O]

24. The method of claim 1, wherein the PSMA-targeting compound comprises a compound of formula (III): 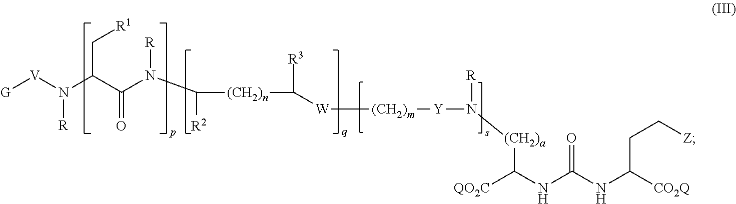
wherein:
q and s are each independently 0 or 1;
p is 0, 1, 2, or 3,
a is 1, 2, 3, or 4;
m is 1, 2, 3, 4, 5, or 6;
n is 1, 2, 3, 4, 5 or 6;
Z is tetrazole or CO₂Q;
each Q is independently selected from hydrogen or a protecting group;
V can be present or absent and when is present is selected from —C(O)—, —NRC(O)—, and —NRC(S)—;
W is selected from —NRC(O)—, —NRC(O)NR—, NRC(S)NR—, —NRC(O)O—, —OC(O)NR—, —OC(O)—, —C(O)NR—, or —C(O)O—;
Y is selected from —C(O)—, —NRC(O)—, —NRC(S)—, and —OC(O).
each R is independently H or C₁-C₄ alkyl;
each R¹ is independently H, C₁-C₆ alkyl, C₂-C₁₂ aryl, or C₄-C₁₆ alkylaryl, whereas when p is 2 or 3, each R¹ may be the same or different;
R² and R³ are independently H, CO₂H, or CO₂R⁴, where R⁴ is a C₁-C₆ alkyl, C₂-C₁₂ aryl, or C₄-C₁₆ alkylaryl, wherein when one of R² and R³ is CO₂H or CO₂R⁴, the other is H; and
G is a metal chelating moiety optionally including a chelated metal or an optical dye that emits in the visible or near infrared spectrum, wherein G can include any additional atoms or linkers necessary to attach the metal chelating moiety or optical dye to the rest of the compound.

25. The method of claim 24, wherein the compound of formula (III) has a structure selected from: 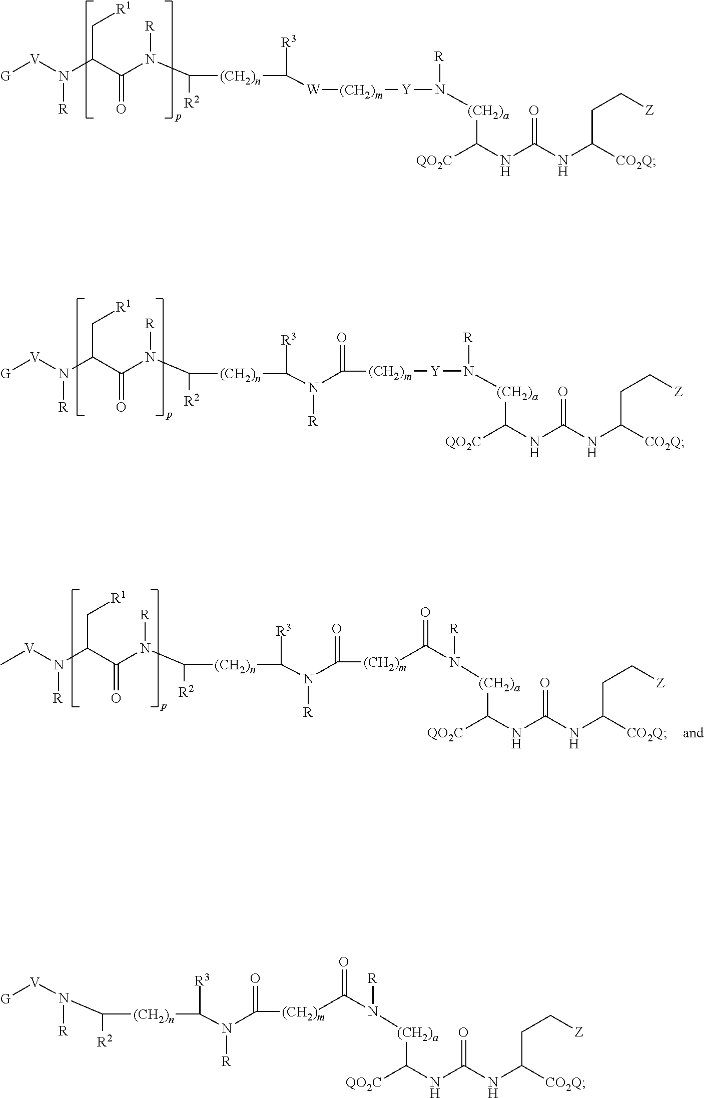
wherein:
Z is tetrazole or CO₂Q;
each Q is independently selected from hydrogen or a protecting group;
a is 1, 2, 3, or 4;
m is 1, 2, 3, 4, 5, or 6;
n is 1, 2, 3, 4, 5 or 6;
p is 0, 1, 2, or 3;
each R is independently H or C₁-C₄ alkyl;
V is selected from —C(O)—, —NRC(O)—, and —NRC(S)—;
W is selected from —NRC(O)—, —NRC(O)NR—, NRC(S)NR—, —NRC(O)O—, —OC(O)NR—, —OC(O)—, —C(O)NR—, and —C(O)O—;
Y is selected from —C(O)—, —NRC(O)—, —NRC(S)—, and —OC(O)—;
R¹ is H, C₁-C₆ alkyl, C₂-C₁₂ aryl, or C₄-C₁₆ alkylaryl, whereas when p is 2 or 3, each R¹ may be the same or different;
R² and R³ are independently H, CO₂H, or CO₂R⁴, where R⁴ is a C₁-C₆ alkyl, C₂-C₁₂ aryl, or C₄-C₁₆ alkylaryl, wherein when one of R² and R³ is CO₂H or CO₂R⁴, the other is H; and
G a metal chelating moiety optionally including a chelated metal or an optical dye that emits in the visible or near infrared spectrum.

26. The method of claim 25, wherein for the compound of formula (III):
R¹ is phenyl;
R⁴ is benzyl;
G is an optical dye; and
(a) R² is H, and R³ is H;
(b) R³ is CO₂H and R² is H;
(c) R² is CO₂H and R³ is H;
(d) R² is CO₂R⁴ and R³ is H; or
(e) R³ is CO₂R⁴, and R² is H.

27. The method of claim 25, wherein G is an optical dye and the compound of formula (III) is selected from:

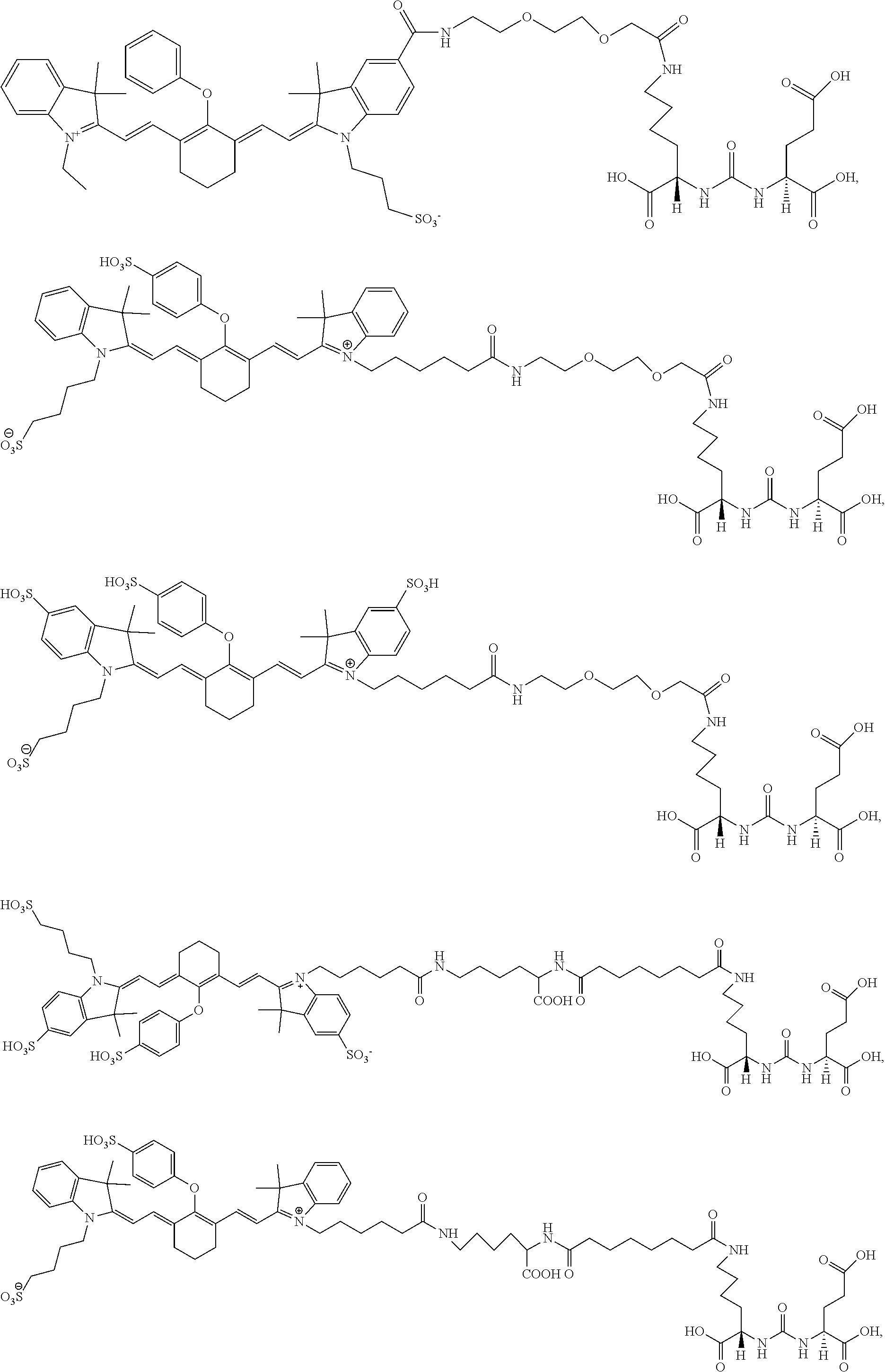

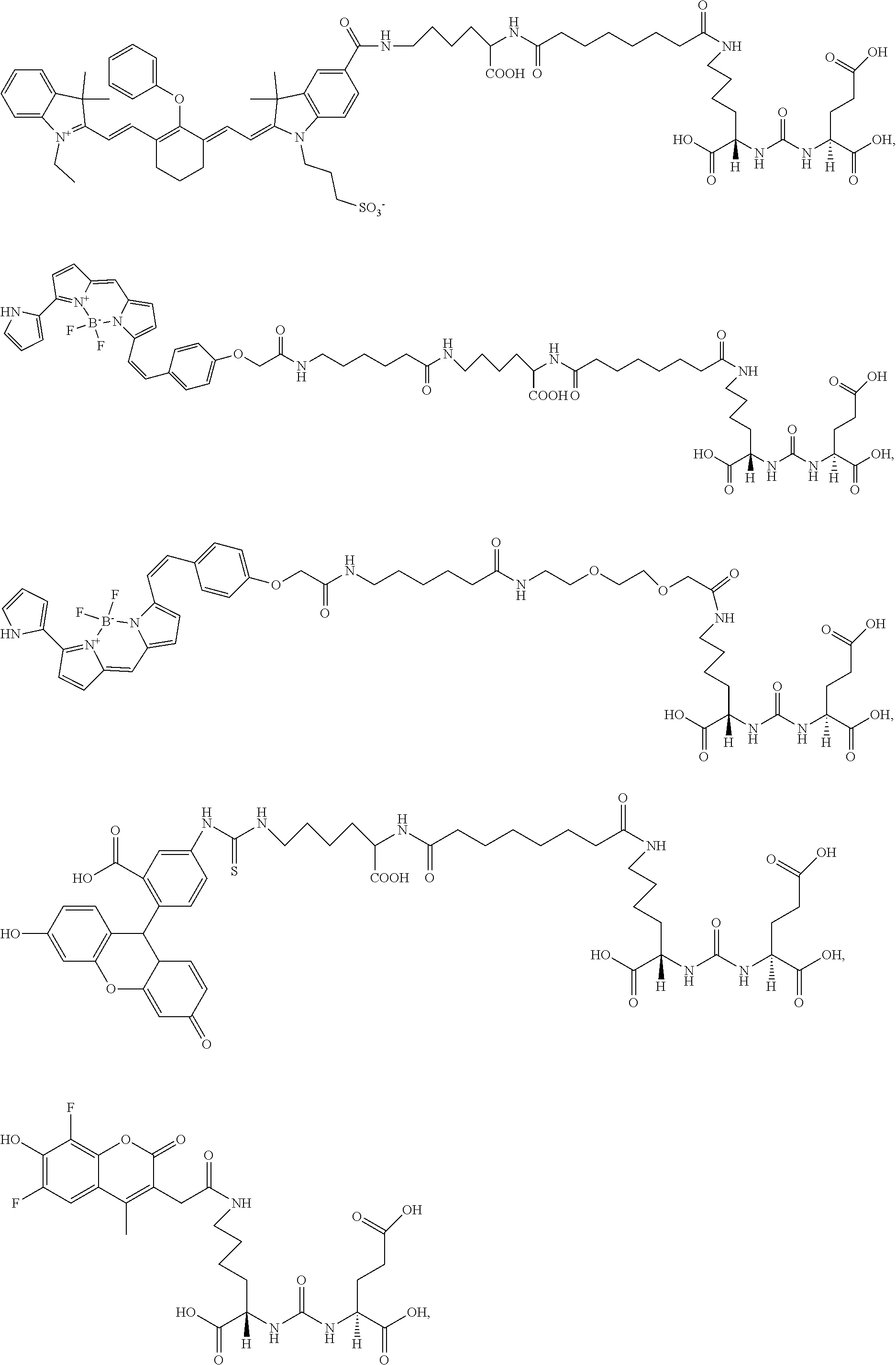

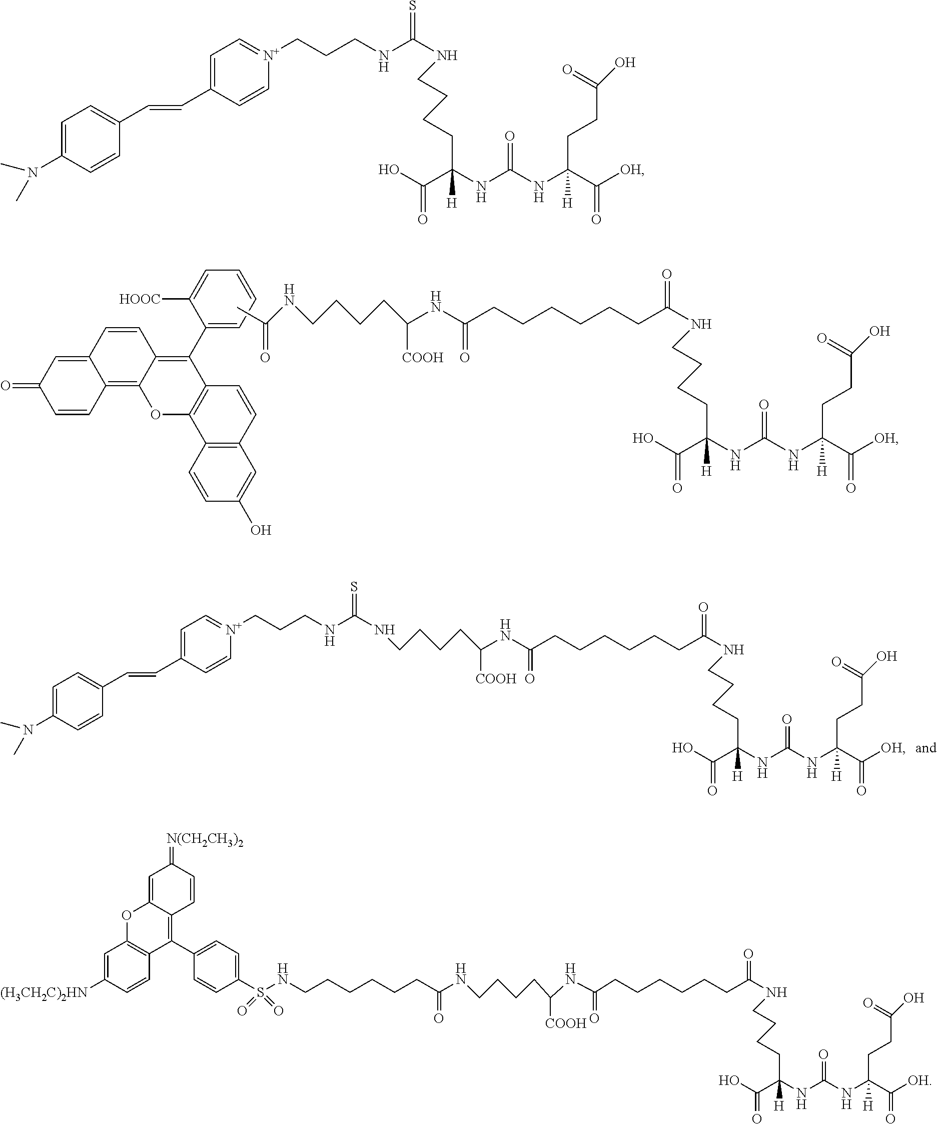

28. The method of claim 25, wherein the compound of formula (III) is:

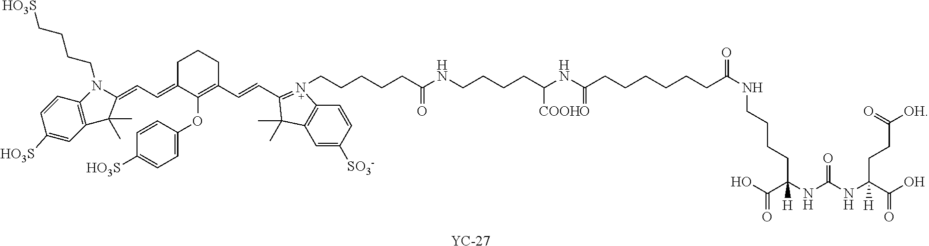

29. The method of claim 24, wherein for the compound of formula (III), wherein the metal chelating moiety is selected from DOTAGA (1,4,7,10-tetraazacyclododececane, 1-(glutaric acid)-4,7,10-triacetic acid), DOTA (1,4,7,10-tetraazacyclododecane-1,4,7,10-tetraacetic acid), DOTASA (1,4,7,10-tetraazacyclododecane-1-(2-succinic acid)-4,7,10-triacetic acid), CB-DO2A (10-bis(carboxymethyl)-1,4,7,10-tetraazabicyclo[5.5.2]tetradecane), DEPA (7-[2-(Bis-carboxymethylamino)-ethyl]-4,10-bis-carboxymethyl-1,4,7,10-tetraaza-cyclododec-1-yl-acetic acid)), 3p-C-DEPA(2-[(carboxymethyl)][5-(4-nitrophenyl-1-[4,7,10-tris(carboxymethyl)-1,4,7,10-tetraazacyclododecan-1-yl]pentan-2-yl)amino]acetic acid)), TCMC (2-(4-isothiocyanotobenzyl)-1,4,7,10-tetraaza-1,4,7,10-tetra-(2-carbamonyl methyl)-cyclododecane), oxo-DO3A (1-oxa-4,7,10-triazacyclododecane-5-S-(4-isothiocyanatobenzyl)-4,7,10-triacetic acid), p-NH₂—Bn-Oxo-DO3A (1-Oxa-4,7,10-tetraazacyclododecane-5-S-(4-aminobenzyl)-4,7,10-triacetic acid), TE2A ((1,8-N,N′-bis-(carboxymethyl)-1,4,8,11-tetraazacyclotetradecane), MM-TE2A, DM-TE2A, CB-TE2A (4,11-bis(carboxymethyl)-1,4,8,11-tetraazabicyclo[6.6.2]hexadecane), CB-TE1A1P (4,8,11-tetraazacyclotetradecane-1-(methanephosphonic acid)-8-(methanecarboxylic acid)), CB-TE2P (1,4,8,11-tetraazacyclotetradecane-1,8-bis(methanephosphonic acid), TETA (1,4,8,11-tetraazacyclotetradecane-1,4,8,11-tetraacetic acid), NOTA (1,4,7-triazacyclononane-N,N′,N″-triacetic acid), NODA (1,4,7-triazacyclononane-1,4-diacetate); NODAGA (1,4,7-triazacyclononane,1-glutaric acid-4,7-acetic acid), (NOTAGA) 1,4,7-triazonane-1,4-diyl)diacetic acid, DFO (Deferoxamine), NETA ([4-[2-(bis-carboxymethylamino)-ethyl]-7-carboxymethl-[1,4,7]triazonan-1-yl}-acetic acid), TACN-TM (N,N′,N″, tris(2-mercaptoethyl)-1,4,7-triazacyclononane), Diamsar (1,8-Diamino-3,6,10,13,16,19-hexaazabicyclo(6,6,6)eicosane, 3,6,10,13,16,19-Hexaazabicyclo[6.6.6]eicosane-1,8-diamine), Sarar (1-N-(4-aminobenzyl)-3, 6,10,13,16,19-hexaazabicyclo[6.6.6]eicosane-1,8-diamine), AmBaSar (4-((8-amino-3,6,10,13,16,19-hexaazabicyclo[6.6.6]icosane-1-ylamino) methyl) benzoic acid), and BaBaSar.

30. The method of claim 24, wherein for the compound of formula (III), the chelating agent is selected from:

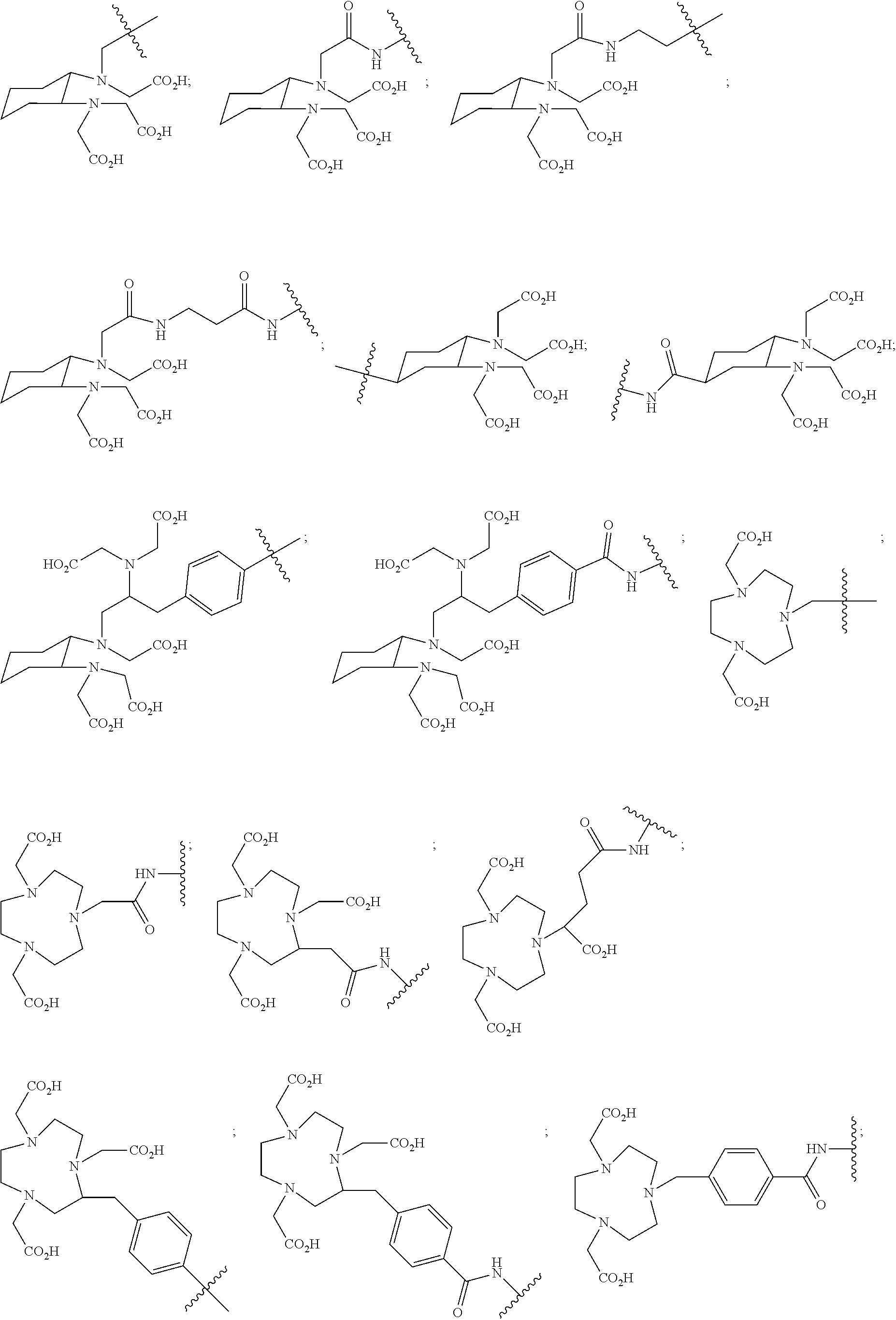

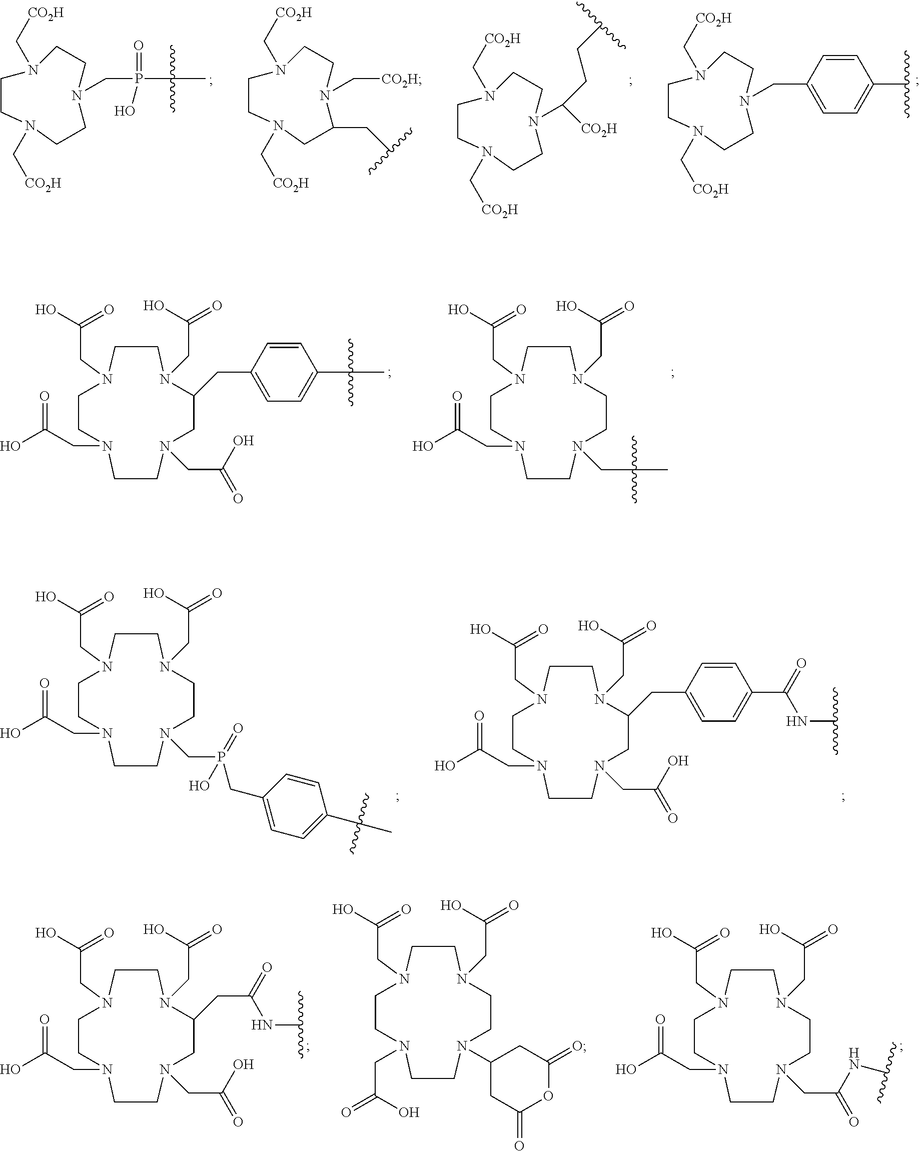

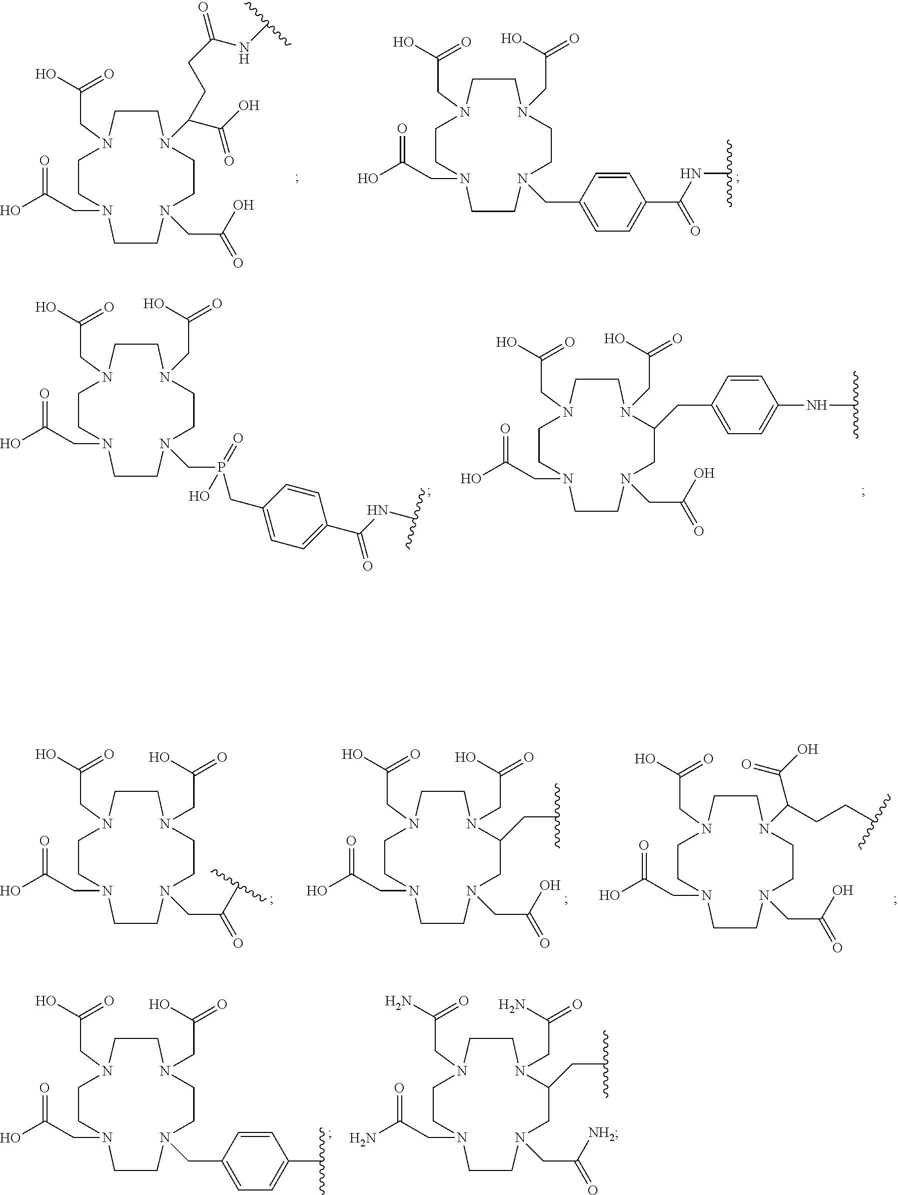

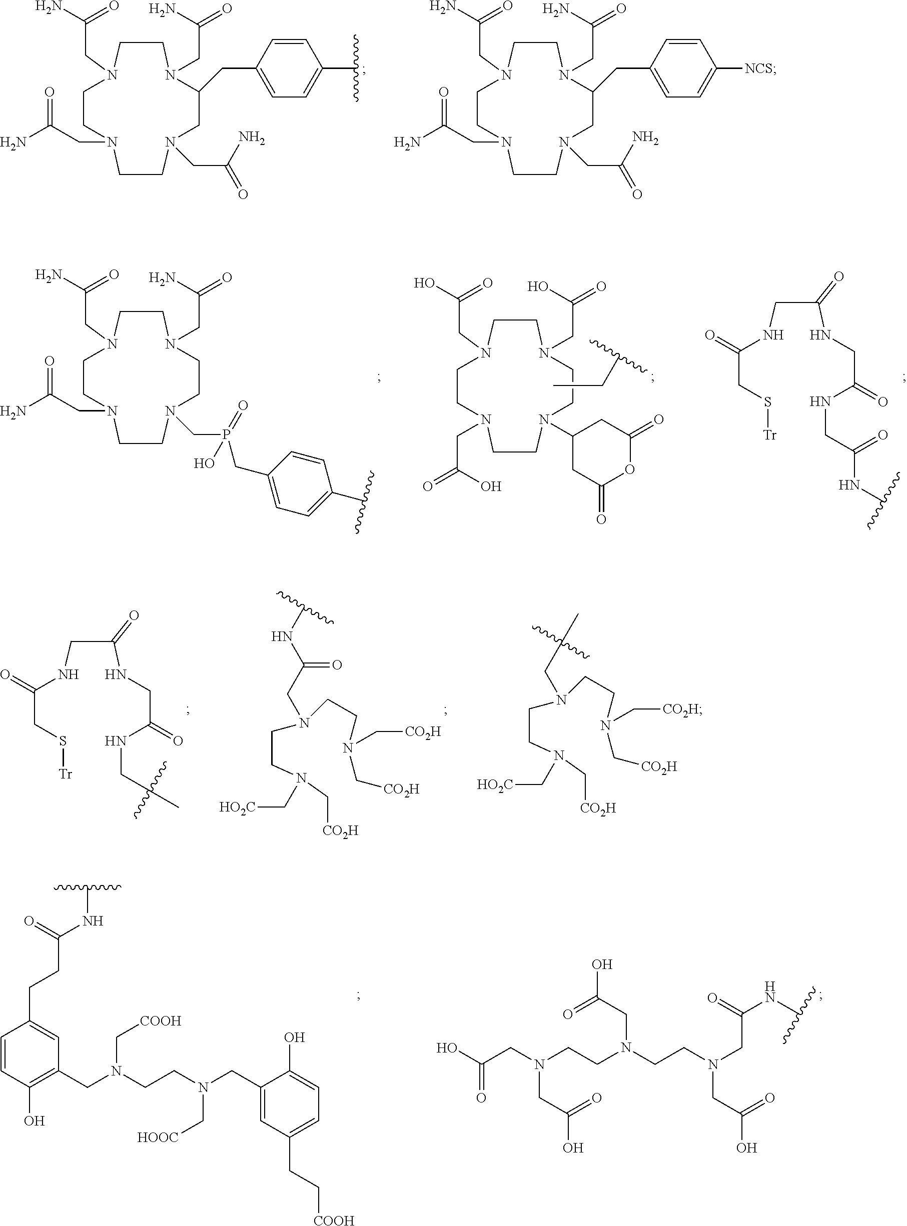

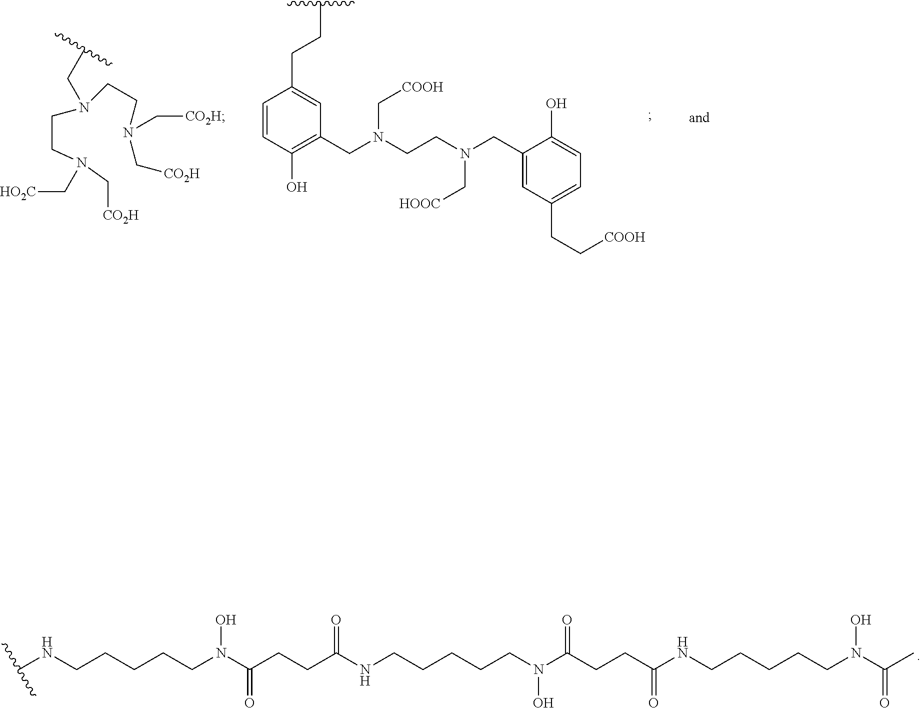 [SMILES: *CN(CCN(CC(=O)O)CC(=O)O)CCN(CC(=O)O)CC(=O)O ; *CCc1ccc(O)c(CN(CCN(CC(=O)O)Cc2cc(CCC(=O)O)ccc2O)CC(=O)O)c1 ; ";" ; *NCCCCCN(O)C(=O)CCC(=O)NCCCCCN(O)C(=O)CCC(=O)NCCCCCN(O)C(C)=O]

31. The method of claim 24, wherein the compound of formula (III) is selected from:

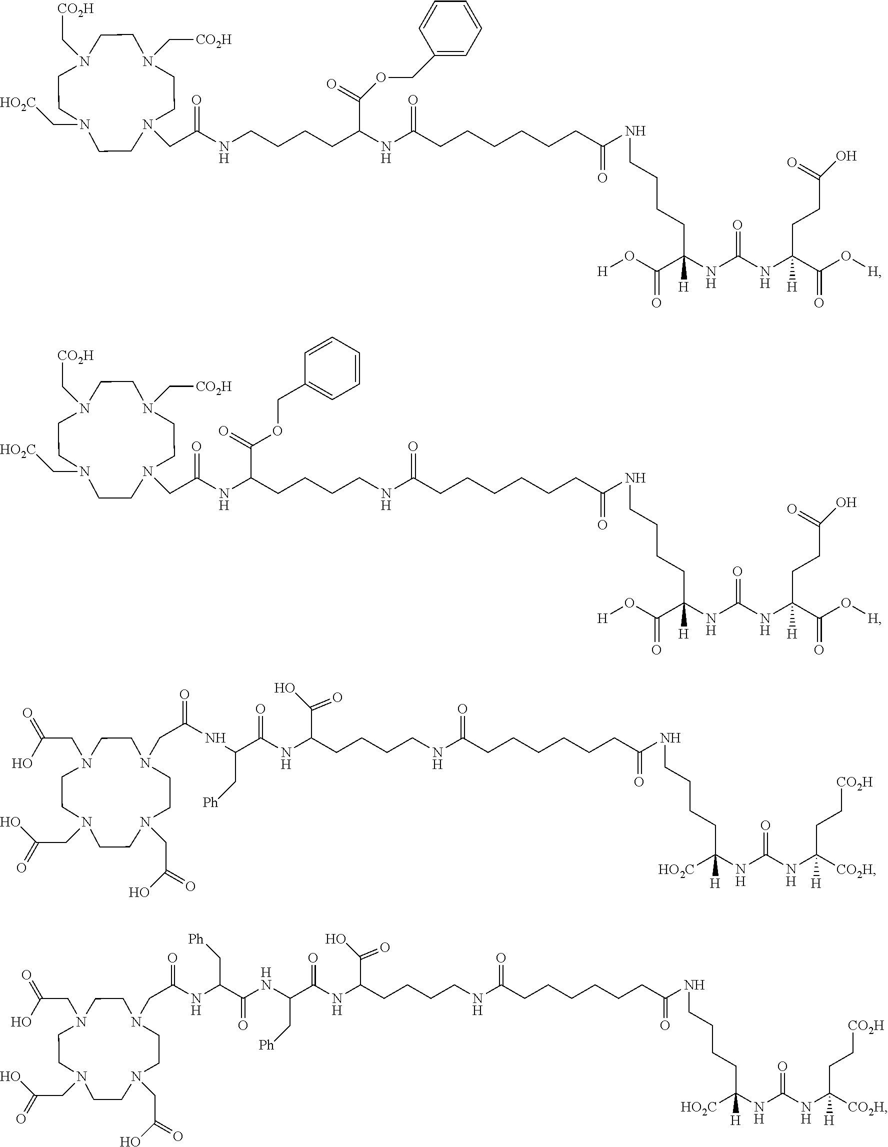

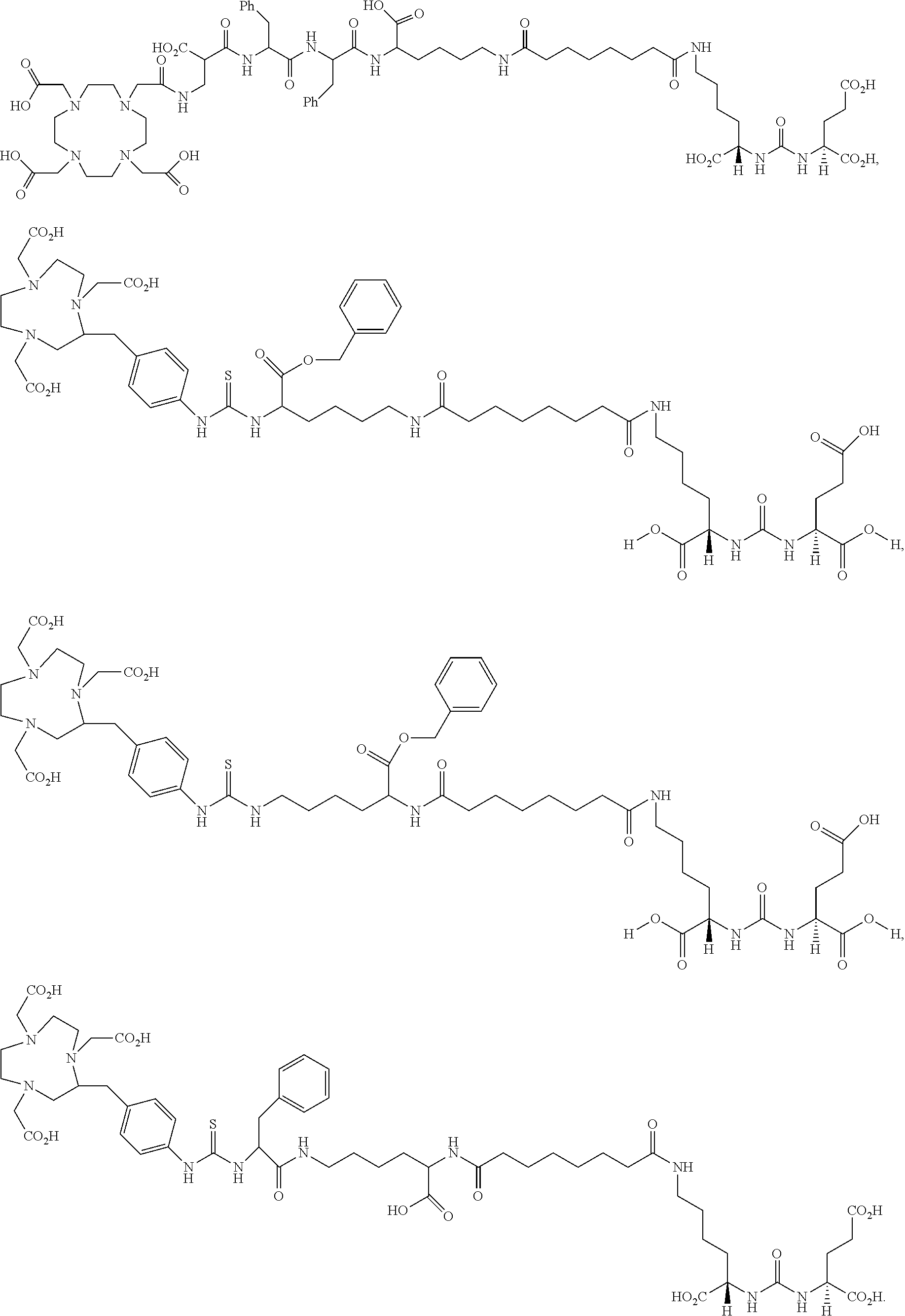

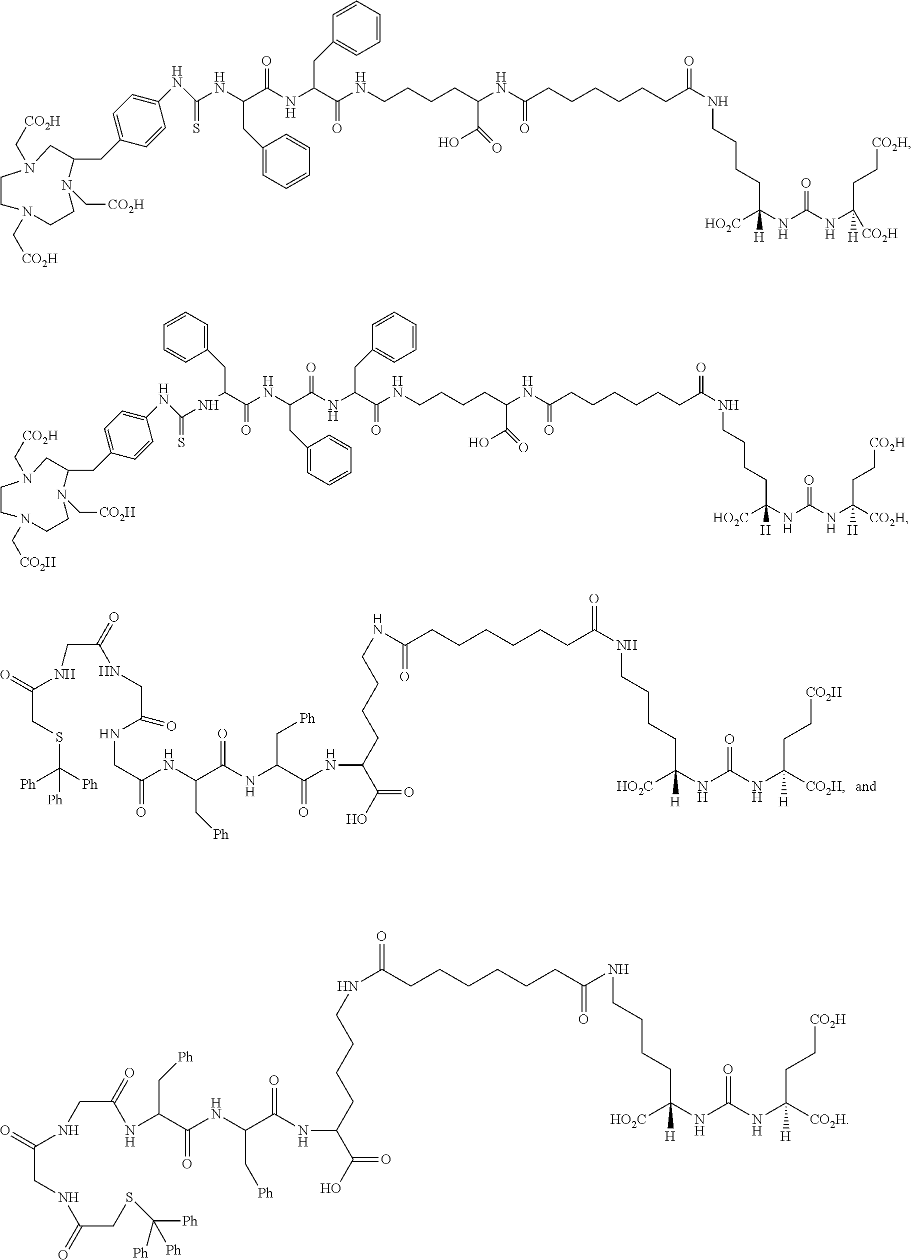

32. The method of claim 24, wherein for the compound of formula (III), the chelated metal is selected from ⁶⁰Cu, ⁶²Cu, ⁶⁴Cu, ⁶⁷Cu, ⁵⁵Co, ⁵⁷Co, ²⁰³Pb, ²¹²Pb, ²²⁵Ac, ¹⁷⁷Lu, ^{99m}Tc, ⁶⁷Ga, ⁶⁸Ga, ¹⁴⁹Tb, ⁸⁶Y, ⁹⁰Y, ¹¹¹In, ¹⁸⁶Re, ¹⁸⁸Re, ¹⁵³Sm, ⁸⁹Zr, ²¹³Bi, ²¹²Bi, ²¹²Pb, ⁶⁷Ga, ⁴⁷Sc, ¹⁶⁶Dy, Al¹⁸F, ¹⁶⁶Ho, and ¹⁷⁷Lu.

33. The method of claim 1, wherein the PSMA-targeting compound is a compound of formula (IV): 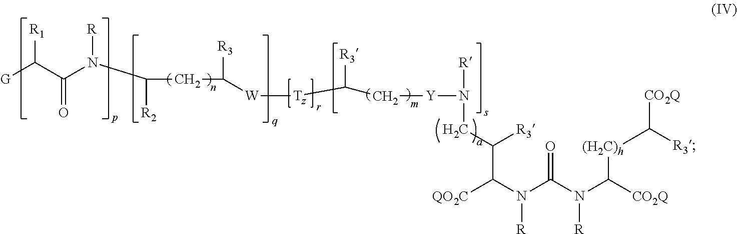
wherein:
each Q is independently hydrogen, a metal ion, a negative charge, or a protecting group;
a, h, m, and n are each independently an integer selected from the group consisting of 1, 2, 3, 4, 5, and 6;
s is 0 or 1;
r is 0 or 1;
q is 0 or 1;
p is an integer selected from the group consisting of 0, 1, 2, and 3, and when p is 2 or 3, each R and R₁ can be the same or different;
each R, R′, and R₁ is independently hydrogen, C₁-C₆ substituted or unsubstituted alkyl, C₆-C₁₂ substituted or unsubstituted aryl, C₆-C₁₂ substituted or unsubstituted heteroaryl, or C₆-C₁₆ alkyaryl;
R₂, R₃, and R₃′ are each independently hydrogen, C₁-C₄ substituted or unsubstituted alkyl, —CO₂H, —CO₂Q, or —CO₂R⁴, wherein R₄ is a C₁-C₆ substituted or unsubstituted alkyl, C₆-C₁₂ substituted or unsubstituted aryl, C₆-C₁₂ substituted or unsubstituted heteroaryl, or C₆-C₁₆ alkyaryl, wherein if one of R₂ and R₃ is —CO₂H, or —CO₂R⁴, then the other is H;
Tz is a triazole containing moiety selected from the group consisting of: 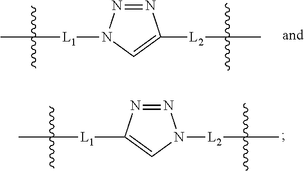
wherein L₁ is 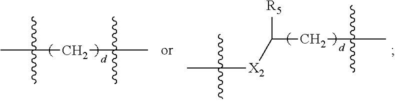
and L₂ is 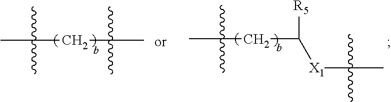
wherein:
X₁ is —NRC(O)—, —NRC(O)NR—, —NRC(S)NR, or —NRC(O)O—;
X₂ is —C(O)NR—, —NRC(O)NR—, —NRC(S)NR—, or —OC(O)NR—;
R₅ is H, —CO₂H, or —CO₂R₆, wherein R₆ is C₁-C₆ alkyl, C₆-C₁₂ aryl, or C₆-C₁₆ alkyaryl; b is 1, 2, 3, or 4; and d is 1, 2, 3, or 4;
Y is —C(O)—, —NRC(O)—, —NRC(S)—, —OC(O)—;
W is a bond, —(CH₂—O)ₜ—, —NRC(O)—, —NRC(O)NR—, —NRC(S)NR—, —NRC(O)O—, —OC(O)NR—, —OC(O)—, —C(O)NR—, or —C(O)O—, wherein t is an integer selected from the group consisting of 1, 2, 3, 4, 5, 6, 7, and 8;
G is 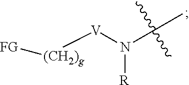
FG is a fluorescent dye moiety that emits in the near-infrared spectrum; V is —C(O)—, —NRC(O)—, —NRC(S)—, or —OC(O)—, and g is an integer selected from the group consisting of 1, 2, 3, 4, 5, and 6;
under the condition that when r is 0, then q and s are both 0 or both 1;
or a pharmaceutically acceptable salt thereof, under the proviso that if R′ is hydrogen, then the fluorescent dye moiety cannot be:

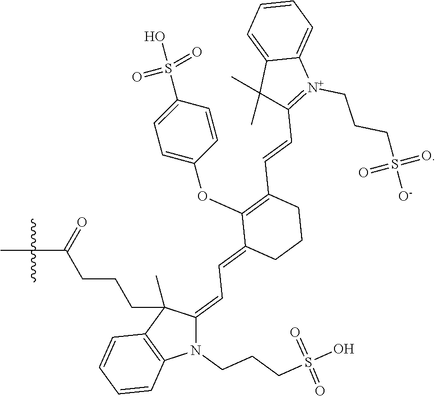 [SMILES: *C(=O)CCCC1(C)/C(=C\C=C2/CCCC(/C=C/C3=[N+](CCCS(=O)(=O)[O-])c4ccccc4C3(C)C)=C2Oc2ccc(S(=O)(=O)O)cc2)N(CCCS(=O)(=O)O)c2ccccc21]

34. The method of claim 33, wherein for the compound of formula (IV), the fluorescent dye moiety is: 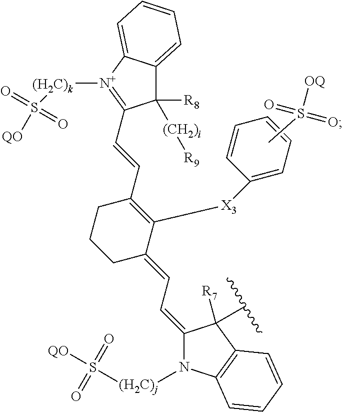
wherein:
i, j, and k are each an integer selected from the group consisting of 1, 2, 3, 4, 5, and 6;
X₃ is a single bond, —O—, or —S—;
R₇, R₈, and R₉ are each independently hydrogen, C₁-C₄ unsubstituted or substituted alkyl, or —CO₂Q;
each Q is independently hydrogen, a metal ion, a negative charge, or a protecting group.

35. The method of claim 33, wherein the compound of formula (IV) is selected from:

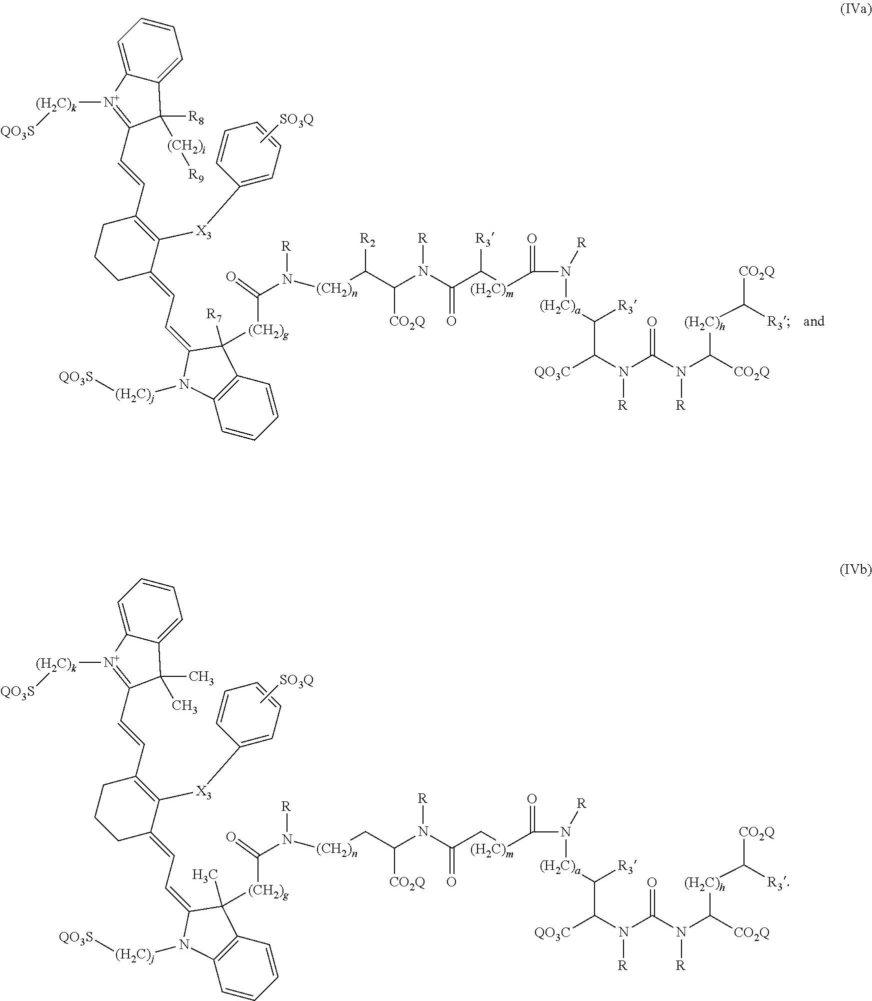

36. The method of claim 35, wherein the compound of formula (IV) is selected from:

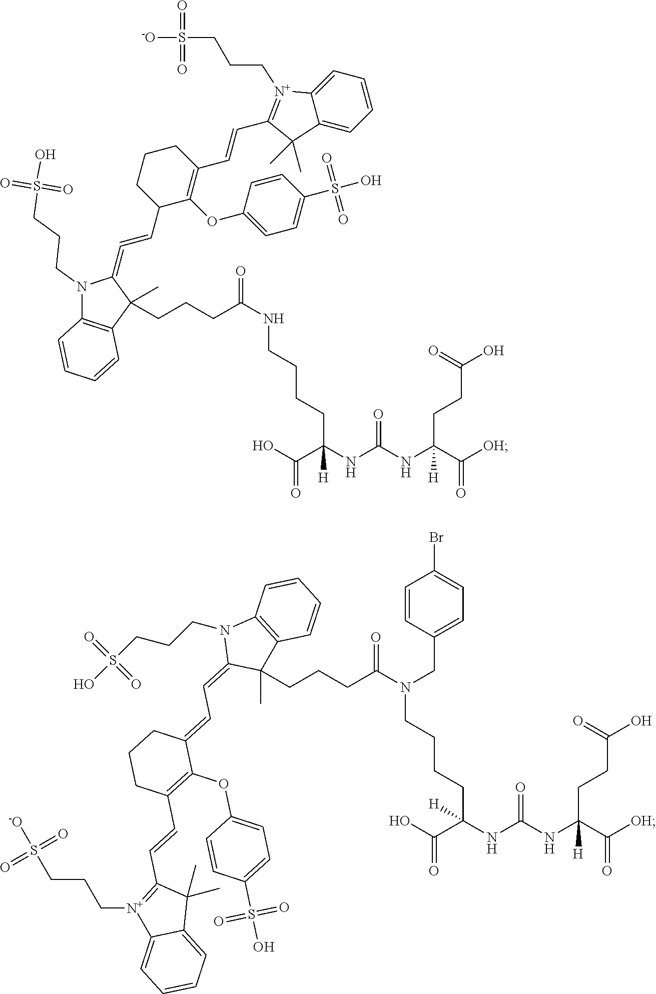

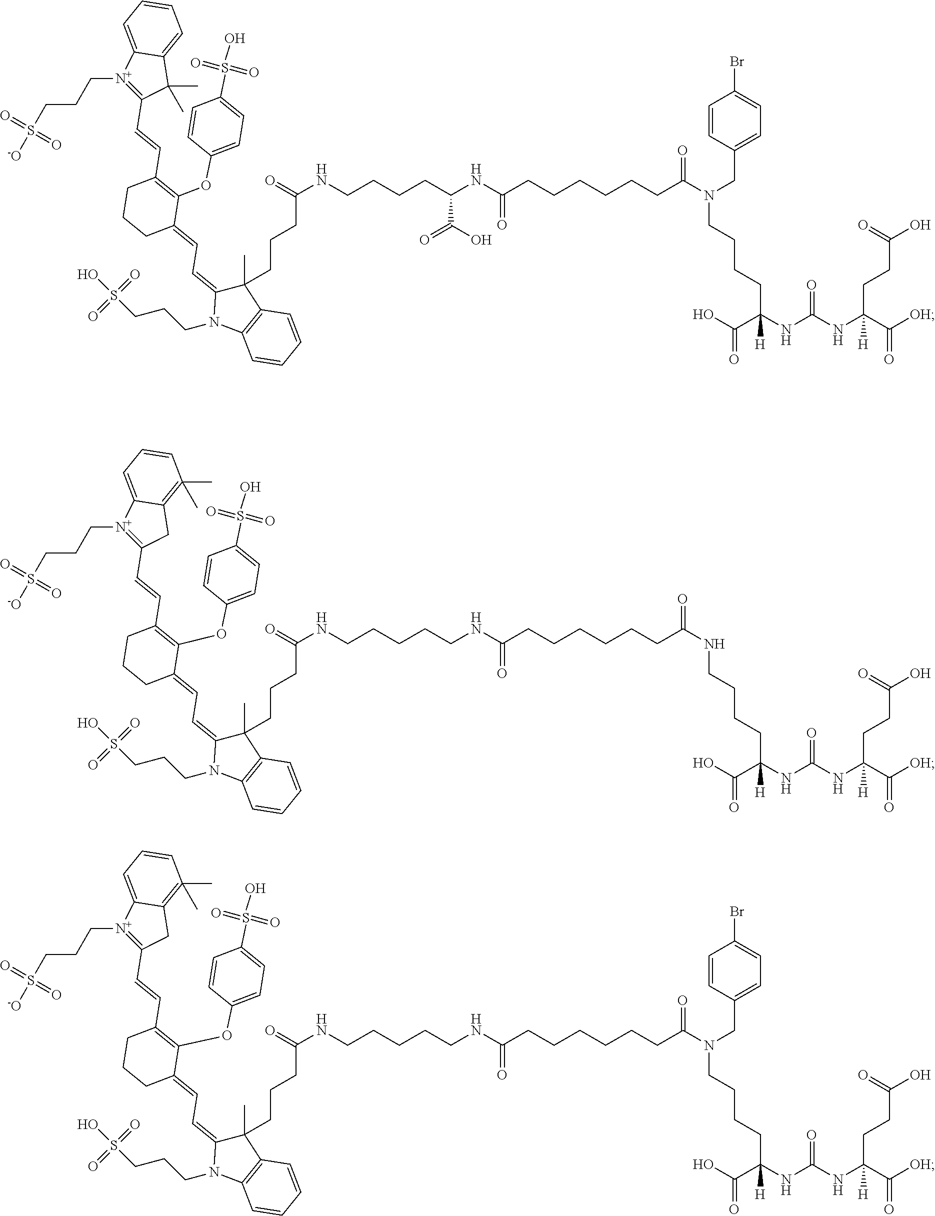

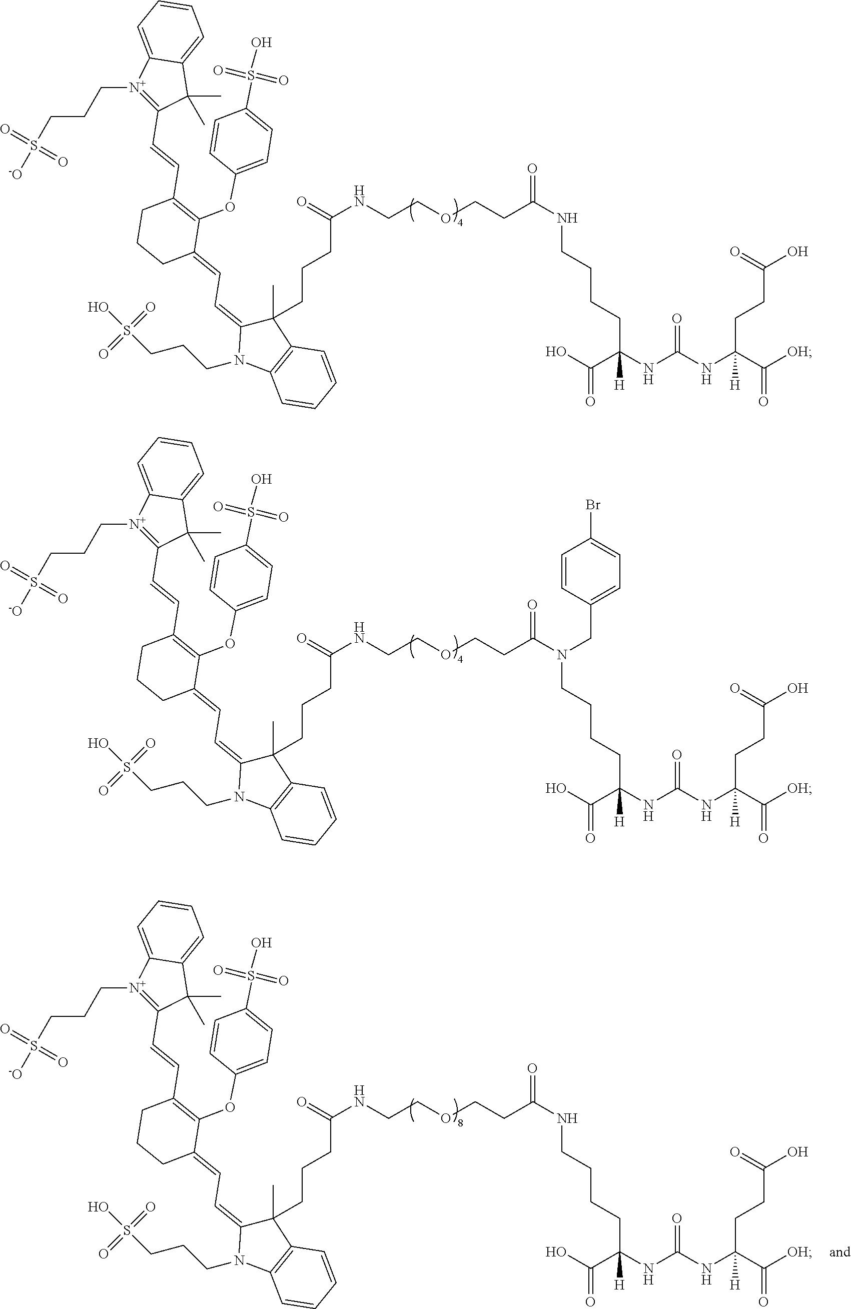

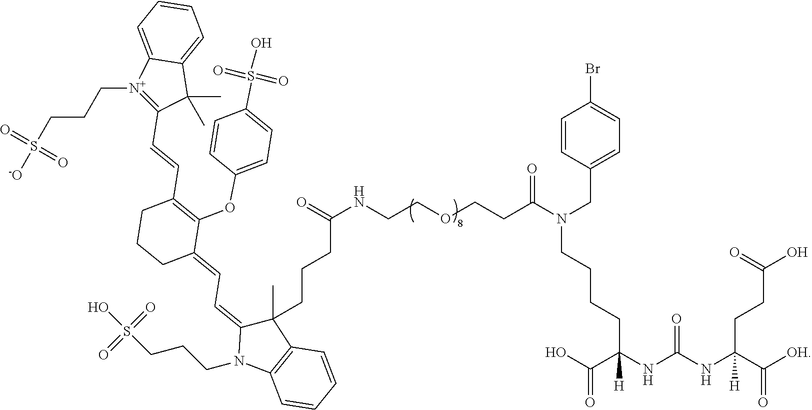

37. The method of claim 1, wherein the PSMA-targeting compound is: [chemical structure]
